(19) United States
(12) Patent Application Publication
(10) Pub. No.: US 2009/0192176 A1
(43) Pub. Date: Jul. 30, 2009

(54) 1H-PYRAZOLO[3,4-D]PYRIMIDINE, PURINE, 7H-PURIN-8(9H)-ONE, 3H-[1,2,3]TRIAZOLO[4,5-D]PYRIMIDINE, AND THIENO[3,2-D]PYRIMIDINE COMPOUNDS, THEIR USE AS mTOR KINASE AND PI3 KINASE INHIBITORS, AND THEIR SYNTHESES

(75) Inventors: Arie Zask, New York, NY (US); Christoph Martin Dehnhardt, New York, NY (US); Joshua Aaron Kaplan, Nyack, NY (US); Efren Guillermo Delos Santos, Nanuet, NY (US); Aranapakam Mudumbai Venkatesan, Rego Park, NY (US); Jeroen Cunera Verheijen, Highland Mills, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/361,607

(22) Filed: Jan. 29, 2009

Related U.S. Application Data
(60) Provisional application No. 61/024,591, filed on Jan. 30, 2008.

(51) Int. Cl. A61K 31/519; C07D 487/04; C07D 495/04; A61P 35/00

(52) U.S. Cl. 514/260.1; 544/256; 544/254; 544/278; 514/261.1; 514/262.1

(57) ABSTRACT

The invention relates to 1H-pyrazolo[3,4-d]pyrimidine, purine, 7H-purin-8(9H)-one, 3H-[1,2,3]triazolo[4,5-d]pyrimidine, and thieno[3,2-d]pyrimidine compounds, compositions comprising the compounds, and methods for making and using the compounds.

### FIELD OF THE INVENTION

The invention relates to 1H-pyrazolo[3,4-d]pyrimidine, purine, 7H-purin-8(9H)-one, 3H-[1,2,3]triazolo[4,5-d]pyrimidine, and thieno[3,2-d]pyrimidine compounds, compositions comprising a 1H-pyrazolo[3,4-d]pyrimidine, purine, 7H-purin-8(9H)-one, 3H-[1,2,3]triazolo[4,5-d]pyrimidine, and thieno[3,2-d]pyrimidine compound, methods of synthesizing these compounds, and methods for treating mTOR-related diseases and treating PI3K-related diseases.

### BACKGROUND OF THE INVENTION

Phosphatidylinositol (hereinafter abbreviated as “PI”) is one of the phospholipids in cell membranes. In recent years it has become clear that PI plays an important role also in intracellular signal transduction. It is well recognized in the art that PI (4,5) bisphosphate (PI(4,5)P2 or PIP2) is degraded into diacylglycerol and inositol (1,4,5) triphosphate by phospholipase C to induce activation of protein kinase C and intracellular calcium mobilization, respectively [M. J. Berridge et al., Nature, 312, 315 (1984); Y. Nishizuka, Science, 225, 1365 (1984)].

In the late 1980s, phosphatidylinositol-3 kinase (“PI3K”) was found to be an enzyme that phosphorylates the 3-position of the inositol ring of phosphatidylinositol [D. Whitman et al., Nature, 332, 664 (1988)]. When PI3K was discovered, it was originally considered to be a single enzyme. Recently however, it was clarified that a plurality of PI3K subtypes exists. Three major subtypes of PI3Ks have now been identified on the basis of their in vitro substrate specificity, and these three are designated class I (a&b), class II, and class III [B. Vanhaesebroeck, Trend in Biol. Sci., 22, 267 (1997)].

The class Ia PI3K subtype has been most extensively investigated to date. Within the class Ia subtype there are three isoforms (α, β, & δ) that exist as hetero dimers of a catalytic 110-kDa subunit and regulatory subunits of 50-85 kDa. The regulatory subunits contain SH2 domains that bind to phosphorylated tyrosine residues within growth factor receptors or adaptor molecules and thereby localize PI3K to the inner cell membrane. At the inner cell membrane PI3K converts PIP2 to PIP3 (phosphatidylinositol-3,4,5-trisphosphate) that serves to localize the downstream effectors PDK1 and Akt to the inner cell membrane where Akt activation occurs. Activated Akt mediates a diverse array of effects including inhibition of apoptosis, cell cycle progression, response to insulin signaling, and cell proliferation. c Class Ia PI3K subtypes also contain Ras binding domains (RBD) that allow association with activated Ras providing another mechanism for PI3K membrane localization. Activated, oncogenic forms of growth factor receptors, Ras, and even PI3K kinase have been shown to aberrantly elevate signaling in the PI3K/Akt/mTOR pathway resulting in cell transformation. As a central component of the PI3K/Akt/mTOR signaling pathway PI3K (particularly the class Ia α isoform) has become a major therapeutic target in cancer drug discovery.

Substrates for class I PI3Ks are PI, PI(4)P and PI(4,5)P2, with PI(4,5)P2 being the most favored. Class I PI3Ks are further divided into two groups, class Ia and class Ib, because of their activation mechanism and associated regulatory subunits. The class Ib PI3K is p110γ that is activated by interaction with G protein-coupled receptors. Interaction between p110γ and G protein-coupled receptors is mediated by regulatory subunits of 110, 87, and 84 kDa.

PI and PI(4)P are the known substrates for class II PI3Ks; PI(4,5)P2 is not a substrate for the enzymes of this class. Class II PI3Ks include PI3K C2α, C2β and C2γ isoforms, which contain C2 domains at the C terminus, implying that their activity is regulated by calcium ions.

The substrate for class III PI3Ks is PI only. A mechanism for activation of the class III PI3Ks has not been clarified. Because each subtype has its own mechanism for regulating activity, it is likely that activation mechanism(s) depend on stimuli specific to each respective class of PI3K.

There are three mTOR inhibitors, which have progressed into clinical trials. These compounds are Wyeth's Torisel, also known as 42-(3-hydroxy-2-(hydroxymethyl)-rapamycin 2-methylpropanoate, CCI-779 or Temsirolimus; Novartis' Everolimus, also known as 42-O-(2-hydroxyethyl)-rapamycin, or RAD 001; and Ariad's AP23573 also known as 42-(dimethylphopsinoyl)-rapamycin. The FDA has approved Torisel for the treatment of advanced renal cell carcinoma. In addition, Torisel is active in a NOS/SCID xenograft mouse model of acute lymphoblastic leukemia [Teachey et al, Blood, 107(3), 1149-1155, 2006]. Everolimus is in a phase II clinical study for patients with Stage 1V Malignant Melanoma. AP23573 has been given orphan drug and fast-track status by the FDA for treatment of soft-tissue and bone sarcomas.

The three mTOR inhibitors have non-linear, although reproducible pharmacokinetic profiles. Mean area under the curve (AUC) values for these drugs increase at a less than dose related way. The three compounds are all semi-synthetic derivatives of the natural macrolide antibiotic rapamycin. It would be desirable to find fully synthetic compounds, which inhibit mTOR that are more potent and exhibit improved pharmacokinetic behaviors.

As explained above, mTOR inhibitors and PI3K inhibitors are expected to be novel types of medicaments useful against cell proliferation disorders, especially as carcinostatic agents. Thus, it would be advantageous to have new mTOR inhibitors and PI3K inhibitors as potential treatment regimens for mTOR- and PI3K-related diseases. The instant invention is directed to these and other important ends.

### SUMMARY OF THE INVENTION

In one aspect, the invention provides compounds of the Formula 1:

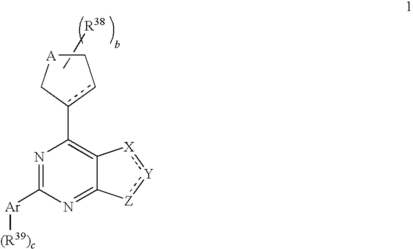

or a pharmaceutically acceptable salt thereof, wherein the constituent variables are as defined below.

In one aspect, the invention provides compounds of the Formula 2:

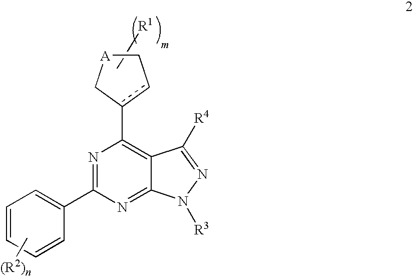

or a pharmaceutically acceptable salt thereof, wherein the constituent variables are as defined below.

In another aspect, the invention provides compounds of the Formula 3:

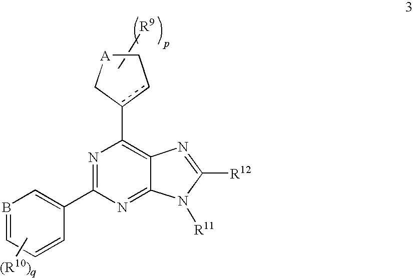

a tautomer thereof, or a pharmaceutically acceptable salt thereof, wherein the constituent variables are as defined below.

In another aspect, the invention provides compounds of the Formula 4:

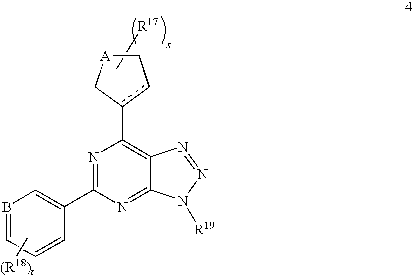

or a pharmaceutically acceptable salt thereof, wherein the constituent variables are as defined below.

In another aspect, the invention provides compounds of the Formula 5:

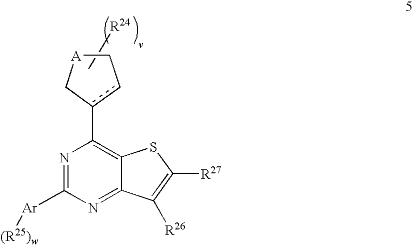

or a pharmaceutically acceptable salt thereof, wherein the constituent variables are as defined below.

In another aspect, the invention provides compounds of the Formula 6:

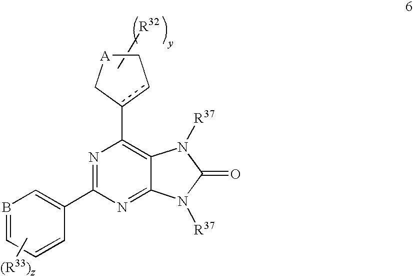

or a pharmaceutically acceptable salt thereof, wherein the constituent variables are as defined below.

In other aspects, the invention provides pharmaceutical compositions comprising compounds or pharmaceutically acceptable salts of compounds of the present invention.

In one aspect, the compounds or pharmaceutically acceptable salts thereof are useful as mTOR inhibitors.

In one aspect, the compounds or pharmaceutically acceptable salts thereof are useful as PI3K inhibitors.

In one aspect, the invention provides methods for treating an mTOR-related disorder, comprising administering to a mammal in need thereof, the compounds or pharmaceutically acceptable salts of compounds of the present invention in an amount effective to treat an mTOR-related disorder.

In one aspect, the invention provides methods for treating a PI3K-related disorder, comprising administering to a mammal in need thereof the compounds or pharmaceutically acceptable salts of compounds of the present invention in an amount effective to treat a PI3K-related disorder.

In other aspects, the invention provides further methods of synthesizing the compounds or pharmaceutically acceptable salts of compounds of the present invention.

### DETAILED DESCRIPTION OF THE INVENTION

In one aspect, the invention provides compounds of the Formula 1:

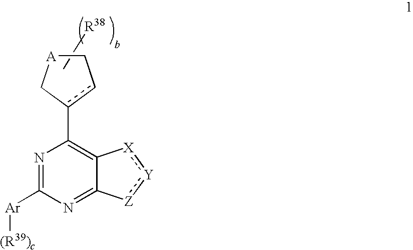

or a pharmaceutically acceptable salt thereof, wherein

A is —O—, —CH₂—O—, —CH₂—CH₂—O—, —CH₂—O—CH₂—, or —CH₂—S—;

the dashed lines - - - - - independently represents an optional second bond;

R³⁸ is independently C₁-C₆alkyl; C₂-C₆alkenyl; C₂-C₆alkynyl; or C₃-C₈cycloalkyl any of which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)C₁-C₆alkyl, C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂;

b is 0, 1, or 2;

Ar is phenyl, naphthyl, or nitrogen-containing mono- or bicyclic heteroaryl;

R³⁹ is independently halogen; one of the meanings of R³⁸; C₁-C₆alkoxy which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₁-C₆alkoxycarbonyl; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; (C₆-C₁₄)aryloxy which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; hydroxyl; NR⁴⁰R⁴¹; NO₂; CN; —C(O)NR⁴⁰R⁴¹; R⁴²C(O)NH—; CO₂H; CF₃; CF₃O; (C₁-C₆alkyl)thio; —SO₂NR⁴⁰R⁴¹; —NHC(O)NR⁴⁰R⁴¹; —NHC(O)OR⁴²; —NH(SO₂)NH—(C₁-C₆alkyl); —NH(SO₂)NH—(C₆-C₁₄aryl); —NHC(S)—NH—(C₁-C₆alkyl); —N═C(S—C₁-C₆alkyl)NH—(C₁-C₆alkyl); —S(O)_{d}—(C₆-C₁₄aryl); —S(O)_{d}—(C₁-C₉heteroaryl); or —N(H)—C(═N—(CN))—O—(C₆-C₁₄aryl);

c is 0, 1, 2, 3, 4, or 5;

each d is independently 1 or 2;

R⁴⁰ and R⁴¹ are each independently H; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, —NO₂, R⁴⁶ or C(O)R⁴⁷; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₃-C₈cycloalkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; 4- to 7-membered monocyclic heterocycle group which is unsubstituted or is substituted with from 1 to 3 substituents selected from C₁-C₈acyl, C₁-C₆alkyl, heterocyclylalkyl, (C₆-C₁₄aryl)alkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂; or R⁴⁰ and R⁴¹ when taken together with the nitrogen to which they are attached can form a 3- to 7-membered nitrogen containing heterocycle wherein up to two of the carbon atoms of the heterocycle can be replaced with —N(R⁴³)—, —O—, or —S(O)_{d}—;

R⁴² is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; or C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂;

R⁴³ is hydrogen; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₃-C₈cycloalkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; amino(C₁-C₆alkyl)-; or arylamino;

X, Y and Z are independently N(R⁴⁴)—; C(R⁴⁵); and S;

R⁴⁴ is hydrogen; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; heterocyclylalkyl; 4- to 7-membered monocyclic heterocycle group which is unsubstituted or is substituted with from 1 to 3 substituents independently selected from C₁-C₈acyl, C₁-C₆alkyl, heterocyclylalkyl, wherein the ring portion of the heterocyclylalkyl group which is unsubstituted or is substituted by 1 to 3 substituents independently selected from halogen, —NH₂, —O(C₁-C₆alkyl), C₁-C₆alkyl, 4- to 7-membered monocyclic heterocycle, and C₃-C₈cycloalkyl, (C₆-C₁₄aryl)alkyl, wherein the ring portion of the (C₆-C₁₄aryl)alkyl group which is unsubstituted or is substituted by 1 to 3 substituents independently selected from halogen, —NH₂, —O(C₁-C₆alkyl), C₁-C₆alkyl, 4- to 7-membered monocyclic heterocycle, and C₃-C₈cycloalkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂; or C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₆-C₁₄)arylalkyl-O—C(O)—, (C₁-C₆alkyl)carboxyamido-, —C(O)NH₂, alkylcarboxamido-, or —NO₂;

R⁴⁵ is hydrogen; or is C₁-C₆alkyl; C₂-C₆alkenyl; or C₂-C₆alkynyl each of which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂;

R⁴⁶ is piperazinyl optionally substituted with 1 or 2 C₁-C₆alkyl; —O(C₂-C₃alkylene)N(C₁-C₆alkyl)(C₁-C₆alkyl); or —(C₁-C₃alkylene)N(C₁-C₆alkyl)(C₁-C₆alkyl); and

R⁴⁷ is piperazinyl optionally substituted with 1 or 2 C₁-C₆alkyl; or —N(C₁-C₃alkyl)-C₂-C₃alkylene-N(C₁-C₆alkyl)(C₁-C₆alkyl).

In one aspect, the invention provides compounds of the Formula 2:

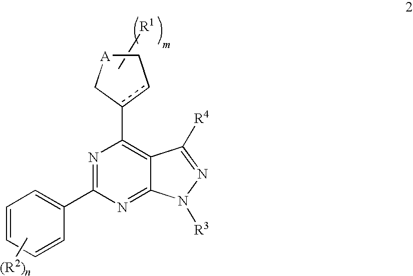

or a pharmaceutically acceptable salt thereof, wherein

A is —O—, —CH₂—O—, —CH₂—CH₂—O—, —CH₂—O—CH₂—, or —CH₂—S—;

the dashed line - - - - - represents an optional second carbon-to-carbon bond;

R¹ is independently C₁-C₆alkyl; C₂-C₆alkenyl; C₂-C₆alkynyl; or C₃-C₈cycloalkyl any of which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)C₁-C₆alkyl, C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂;

m is 0, 1, or 2;

R² is independently halogen; one of the meanings of R¹; C₁-C₆alkoxy which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₁-C₆alkoxycarbonyl; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; (C₆-C₁₄)aryloxy which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; hydroxyl; NR⁵R⁶; NO₂; CN; —C(O)NR⁵R⁶; R⁷C(O)NH—; CO₂H; CF₃; CF₃O; (C₁-C₆alkyl)thio; —SO₂NR⁵R⁶; —NHC(O)NR⁵R⁶; —NHC(O)OR⁷; —NH(SO₂)NH—(C₁-C₆alkyl); —NH(SO₂)NH—(C₆-C₁₄aryl); —NHC(S)—NH—(C₁-C₆alkyl); —N═C(S—C₁-C₆alkyl)NH—(C₁-C₆alkyl); —S(O)ₖ—(C₆-C₁₄aryl); S(O)ₖ—(C₁-C₉heteroaryl); or —N(H)—C(═N—(CN))—O—(C₆-C₁₄aryl);

n is 0, 1, 2, 3, 4, or 5;

each k is independently 1 or 2;

R⁵ and R⁶ are each independently H; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, —NO₂, R⁴⁶ or C(O)R⁴⁷; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₃-C₈cycloalkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; 4- to 7-membered monocyclic heterocycle group which is unsubstituted or is substituted with from 1 to 3 substituents selected from C₁-C₈acyl, C₁-C₆alkyl, heterocyclylalkyl, (C₆-C₁₄aryl)alkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂; or R⁵ and R⁶ when taken together with the nitrogen to which they are attached can form a 3- to 7-membered nitrogen containing heterocycle wherein up to two of the carbon atoms of the heterocycle can be replaced with —N(R⁸)—, —O—, or —S(O)ₖ—;

R⁷ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; or C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂;

R⁸ is hydrogen; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₃-C₈cycloalkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; amino(C₁-C₆alkyl)-; or arylamino;

R³ is hydrogen; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; heterocyclylalkyl; 4- to 7-membered monocyclic heterocycle group which is unsubstituted or is substituted with from 1 to 3 substituents independently selected from C₁-C₈acyl, C₁-C₆alkyl, heterocyclylalkyl, wherein the ring portion of the heterocyclylalkyl group which is unsubstituted or is substituted by 1 to 3 substituents independently selected from halogen, —NH₂, —O(C₁-C₆alkyl), C₁-C₆alkyl, 4- to 7-membered monocyclic heterocycle, and C₃-C₈cycloalkyl, (C₆-C₁₄aryl)alkyl, wherein the ring portion of the (C₆-C₁₄aryl)alkyl group which is unsubstituted or is substituted by 1 to 3 substituents independently selected from halogen, —NH₂, —O(C₁-C₆alkyl), C₁-C₆alkyl, 4- to 7-membered monocyclic heterocycle, and C₃-C₈cycloalkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂; or C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₆-C₁₄)arylalkyl-O—C(O)—, (C₁-C₆alkyl)carboxyamido-, —C(O)NH₂, alkylcarboxamido-, or —NO₂;

R⁴ is hydrogen; or is C₁-C₆alkyl; C₂-C₆alkenyl; or C₂-C₆alkynyl each of which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂;

R⁴⁶ is piperazinyl optionally substituted with 1 or 2 C₁-C₆alkyl; —O(C₂-C₃alkylene)N(C₁-C₆alkyl)(C₁-C₆alkyl); or —(C₁-C₃alkylene)N(C₁-C₆alkyl)(C₁-C₆alkyl); and

R⁴⁷ is piperazinyl optionally substituted with 1 or 2 C₁-C₆alkyl; or —N(C₁-C₃alkyl)-C₂-C₃alkylene-N(C₁-C₆alkyl)(C₁-C₆alkyl).

In another aspect, the invention provides compounds of the Formula 3:

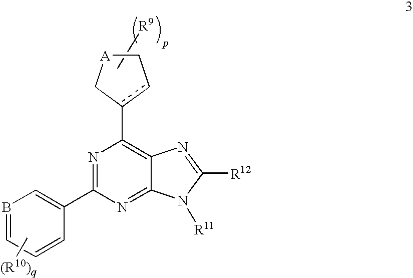

a tautomer thereof, or a pharmaceutically acceptable salt thereof, wherein

A is —O—, —CH₂—O—, —CH₂—CH₂—O—, —CH₂—O—CH₂—, or —CH₂—S—;

the dashed line - - - - - represents an optional second carbon to carbon bond;

R⁹ is C₁-C₆alkyl; C₂-C₆alkenyl; C₂-C₆alkynyl; or C₃-C₈cycloalkyl each of which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)C₁-C₆alkyl, C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂;

p is 0, 1, or 2;

B is N or CH;

R¹⁰ is independently halogen; one of the meanings of R⁹; C₁-C₆alkoxy which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₁-C₆alkoxycarbonyl; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; (C₆-C₁₄)aryloxy which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; hydroxyl; NR¹³R¹⁴; NO₂; CN; —C(O)NR¹³R¹⁴; R¹⁵C(O)NH—; CO₂H; CF₃; CF₃O; (C₁-C₆alkyl)thio; —SO₂NR¹³R¹⁴; —NHC(O)NR¹³R¹⁴; —NHC(O)OR¹⁵; —NH(SO₂)NH—(C₁-C₆alkyl); —NH(SO₂)NH—(C₆-C₁₄aryl); —NHC(S)—NH—(C₁-C₆alkyl); —N═C(S—C₁-C₆alkyl)NH—(C₁-C₆alkyl); —S(O)ᵣ—(C₆-C₁₄aryl); S(O)ᵣ—(C₁-C₉heteroaryl); or —N(H)—C(═N—(CN))—O—(C₆-C₁₄aryl);

q is 0, 1, 2, 3, 4, or 5;

each r is independently 1 or 2;

R¹³ and R¹⁴ are each independently H; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, —NO₂, R⁴⁶ or C(O)R⁴⁷; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; 4- to 7-membered monocyclic heterocycle group which is unsubstituted or is substituted with from 1 to 3 substituents selected from C₁-C₈acyl, C₁-C₆alkyl, heterocyclylalkyl, (C₆-C₁₄aryl)alkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂; or R¹³ and R¹⁴ when taken together with the nitrogen to which they are attached can form a 3- to 7-membered nitrogen containing heterocycle wherein up to two of the carbon atoms of the heterocycle can be replaced with —N(R¹⁶)—, —O—, or —S(O)ᵣ—;

R¹⁵ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; or C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂;

R¹⁶ is hydrogen; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₃-C₈cycloalkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; amino(C₁-C₆alkyl)-; or arylamino;

R¹¹ is hydrogen; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; heterocyclylalkyl; 4- to 7-membered monocyclic heterocycle group which is unsubstituted or is substituted with from 1 to 3 substituents independently selected from C₁-C₈acyl, C₁-C₆alkyl, heterocyclylalkyl, wherein the ring portion of the heterocyclylalkyl group is unsubstituted or is substituted by 1 to 3 substituents independently selected from halogen, —NH₂, —O(C₁-C₆alkyl), C₁-C₆alkyl, 4- to 7-membered monocyclic heterocycle, and C₃-C₈cycloalkyl, (C₆-C₁₄aryl)alkyl, wherein the ring portion of the (C₆-C₁₄aryl)alkyl group is unsubstituted or is substituted by 1 to 3 substituents independently selected from halogen, —NH₂, —O(C₁-C₆alkyl), C₁-C₆alkyl, 4- to 7-membered monocyclic heterocycle, and C₃-C₈cycloalkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂; or C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₆-C₁₄)arylalkyl-O—C(O)—, (C₁-C₆alkyl)carboxyamido-, —C(O)NH₂, alkylcarboxamido-, or —NO₂;

R¹² is H or hydroxyl;

R⁴⁶ is piperazinyl optionally substituted with 1 or 2 C₁-C₆alkyl; —O(C₂-C₃alkylene)N(C₁-C₆alkyl)(C₁-C₆alkyl); or —(C₁-C₃alkylene)N(C₁-C₆alkyl)(C₁-C₆alkyl); and

R⁴⁷ is piperazinyl optionally substituted with 1 or 2 C₁-C₆alkyl; or —N(C₁-C₃alkyl)-C₂-C₃alkylene-N(C₁-C₆alkyl)(C₁-C₆alkyl).

In another aspect, the invention provides compounds of the Formula 4:

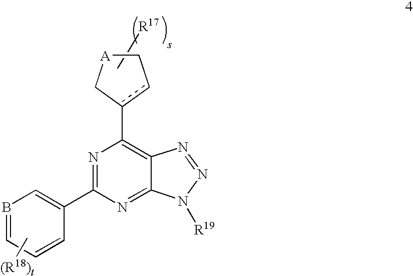

or a pharmaceutically acceptable salt thereof, wherein

A is —O—, —CH₂—O—, —CH₂—CH₂—O—, —CH₂—O—CH₂—, or —CH₂—S—;

the dashed line - - - - - represents an optional second carbon to carbon bond;

R¹⁷ is C₁-C₆alkyl; C₂-C₆alkenyl; C₂-C₆alkynyl; or C₃-C₈cycloalkyl each of which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)C₁-C₆alkyl, C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂;

s is 0, 1, or 2;

B is N or CH;

R¹⁸ is independently halogen; one of the meanings of R¹⁷; C₁-C₆alkoxy which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₆cycloalkyl, halo(C₁-C₆alkyl)-, amino(C₁-C₆alkyl)-, —OC(O)(C₁-C₆alkyl), carboxyamidoalkyl-, or —NO₂; C₁-C₆alkoxycarbonyl; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; (C₆-C₁₄)aryloxy which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, di(C₁-C₆alkyl)amino-, —COOH, —C(O)O—(C₁-C₆alkyl), —OC(O)(C₁-C₆alkyl), N—(C₁-C₆alkyl)amido-, —C(O)NH₂, alkylcarboxamido-, or —NO₂; hydroxyl; NR²⁰R²¹; NO₂; CN; —C(O)NR²⁰R²¹; R²²C(O)NH—; CO₂H; CF₃; CF₃O; C₁-C₆alkylthio; SO₂NR²⁰R²¹; NHC(O)NR²⁰R²¹; —NHC(O)OR²²; —NH(SO₂)NH—(C₁-C₆alkyl); —NH(SO₂)NH—(C₆-C₁₄aryl); —NHC(S)—NH—(C₁-C₆alkyl); —N═C(S—C₁-C₆alkyl)NH—(C₁-C₆alkyl); —S(O)ᵤ—(C₆-C₁₄aryl); —S(O)ᵤ—(C₁-C₉heteroaryl); or —N(H)—C(═N—(CN))—O—(C₆-C₁₄aryl);

t is 0, 1, 2, 3, 4, or 5;

each u is independently 1 or 2;

R²⁰ and R²¹ are each independently H; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, —NO₂, R⁴⁶ or C(O)R⁴⁷; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₃-C₈cycloalkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; 4- to 7-membered monocyclic heterocycle group which is unsubstituted or is substituted with from 1 to 3 substituents selected from C₁-C₈acyl, C₁-C₆alkyl, heterocyclylalkyl, (C₆-C₁₄aryl)alkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂; or R²⁰ and R²¹ when taken together with the nitrogen to which they are attached can form a 3- to 7-membered nitrogen containing heterocycle wherein up to two of the carbon atoms of the heterocycle can be replaced with —N(R²³)—, —O—, or —S(O)ᵤ—;

R²² is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; or C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂;

R²³ is hydrogen; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₃-C₈cycloalkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; amino(C₁-C₆alkyl)-; or arylamino;

R¹⁹ is hydrogen; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; heterocyclylalkyl; 4- to 7-membered monocyclic heterocycle group which is unsubstituted or is substituted with from 1 to 3 substituents independently selected from C₁-C₈acyl, C₁-C₆alkyl, heterocyclylalkyl, wherein the ring portion of the heterocyclylalkyl group is unsubstituted or is substituted by 1 to 3 substituents independently selected from halogen, —NH₂, —O(C₁-C₆alkyl), C₁-C₆alkyl, 4- to 7-membered monocyclic heterocycle, and C₃-C₈cycloalkyl, (C₆-C₁₄aryl)alkyl, wherein the ring portion of the (C₆-C₁₄aryl)alkyl group is unsubstituted or is substituted by 1 to 3 substituents independently selected from halogen, —NH₂, —O(C₁-C₆alkyl), C₁-C₆alkyl, 4- to 7-membered monocyclic heterocycle, and C₃-C₈cycloalkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂; or C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂;

R⁴⁶ is piperazinyl optionally substituted with 1 or 2 C₁-C₆alkyl; —O(C₂-C₃alkylene)N(C₁-C₆alkyl)(C₁-C₆alkyl); or —(C₁-C₃alkylene)N(C₁-C₆alkyl)(C₁-C₆alkyl); and

R⁴⁷ is piperazinyl optionally substituted with 1 or 2 C₁-C₆alkyl; or —N(C₁-C₃alkyl)-C₂-C₃alkylene-N(C₁-C₆alkyl)(C₁-C₆alkyl).

In another aspect, the invention provides compounds of the Formula 5:

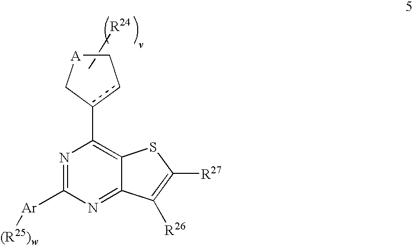

or a pharmaceutically acceptable salt thereof, wherein

A is —O—, —CH₂—O—, —CH₂—CH₂—O—, —CH₂—O—CH₂—, or —CH₂—S—;

the dashed line - - - - - represents an optional second carbon to carbon bond;

R²⁴ is C₁-C₆alkyl; C₂-C₆alkenyl; C₂-C₆alkynyl; or C₃-C₈cycloalkyl each of which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)C₁-C₆alkyl, C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂;

v is 0, 1, or 2;

Ar is phenyl, naphthyl, or nitrogen-containing mono- or bicyclic heteroaryl;

R²⁵ is independently halogen; one of the meanings of R²⁴; C₁-C₆alkoxy which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₁-C₆alkoxycarbonyl; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; hydroxyl; NR²⁸R²⁹; NO₂; CN; —C(O)NR²⁸R²⁹; R³⁰C(O)NH—; CO₂H; CF₃; CF₃O; C₁-C₆alkylthio; —SO₂NR²⁸R²⁹; —NHC(O)NR²⁸R²⁹; —NHC(O)OR³⁰; —NH(SO₂)NH—(C₁-C₆alkyl); —NH(SO₂)NH—(C₆-C₁₄aryl); —NHC(S)—NH—(C₁-C₆alkyl); —N═C(S—(C₁-C₆alkyl))(NH—(C₁-C₆alkyl)); —S(O), —(C₆-C₁₄aryl); —S(O), —(C₁-C₉heteroaryl); or —N(H)—C(═N—(CN))—(O—(C₆-C₁₄aryl));

w is 0, 1, 2, 3, 4, or 5;

each x is independently 1 or 2;

R²⁸ and R²⁹ are each independently H; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, —NO₂, R⁴⁶ or C(O)R⁴⁷; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₃-C₈cycloalkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; 4- to 7-membered monocyclic heterocycle group which is unsubstituted or is substituted with from 1 to 3 substituents selected from C₁-C₈acyl, C₁-C₆alkyl, heterocyclylalkyl, (C₆-C₁₄aryl)alkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂; or R²⁸ and R²⁹ when taken together with the nitrogen to which they are attached can form a 3- to 7-membered nitrogen containing heterocycle wherein up to two of the carbon atoms of the heterocycle can be replaced with —N(R³¹)—, —O—, or —S(O)ₓ—;

R³⁰ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; or C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂;

R³¹ is hydrogen; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₃-C₈cycloalkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; amino(C₁-C₆alkyl)-; or arylamino;

R²⁶ and R²⁷ independently are hydrogen; or are C₁-C₆alkyl; C₂-C₆alkenyl; or C₂-C₆alkynyl each of which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂;

R⁴⁶ is piperazinyl optionally substituted with 1 or 2 C₁-C₆alkyl; —O(C₂-C₃alkylene)N(C₁-C₆alkyl)(C₁-C₆alkyl); or —(C₁-C₃alkylene)N(C₁-C₆alkyl)(C₁-C₆alkyl); and

R⁴⁷ is piperazinyl optionally substituted with 1 or 2 C₁-C₆alkyl; or —N(C₁-C₃alkyl)-C₂-C₃alkylene-N(C₁-C₆alkyl)(C₁-C₆alkyl).

In another aspect, the invention provides compounds of the Formula 6:

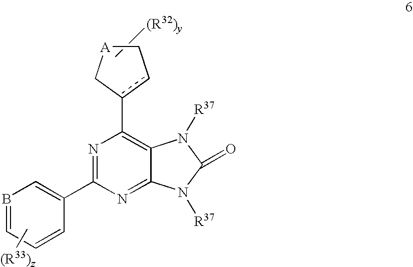

or a pharmaceutically acceptable salt thereof, wherein

A is —O—, —CH₂—O—, —CH₂—CH₂—O—, —CH₂—O—CH₂—, or —CH₂—S—;

the dashed line - - - - - represents an optional second carbon to carbon bond;

R³² is independently C₁-C₆alkyl; C₂-C₆alkeny; C₂-C₆alkynyl; or C₃-C₈cycloalkyl each of which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)C₁-C₆alkyl, C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂;

y is 0, 1, or 2;

B is N or CH;

R³³ is independently halogen; one of the meanings of R³²; C₁-C₆alkoxy which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₁-C₆alkoxycarbonyl; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; hydroxyl; NR³⁴R³⁵; NO₂; CN; —C(O)NR³⁴R³⁵; R³⁶C(O)NH—; CO₂H; CF₃; CF₃O; C₁-C₆alkylthio; —SO₂NR³⁴R³⁵; —NHC(O)NR³⁴R³⁵; —NHC(O)OR³⁶; —NH(SO₂)NH—(C₁-C₆alkyl); —NH(SO₂)NH—(C₆-C₁₄aryl); —NHC(S)—NH—(C₁-C₆alkyl); —N═C(S—(C₁-C₆alkyl))(NH—(C₁-C₆alkyl)); —S(O)ₐ—(C₆-C₁₄aryl); —S(O)ₐ—(C₁-C₉heteroaryl); or —N(H)—C(═N—(CN))—(O—(C₆-C₁₄aryl));

z is 0, 1, 2, 3, 4, or 5;

each a is independently 1 or 2;

R³⁴ and R³⁵ are each independently H; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, —NO₂, R⁴⁶ or C(O)R⁴⁷; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₃-C₈cycloalkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; 4- to 7-membered monocyclic heterocycle group which is unsubstituted or is substituted with from 1 to 3 substituents selected from C₁-C₈acyl, C₁-C₆alkyl, heterocyclylalkyl, (C₆-C₁₄aryl)alkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂; or R³⁴ and R³⁵ when taken together with the nitrogen to which they are attached can form a 3- to 7-membered nitrogen containing heterocycle wherein up to two of the carbon atoms of the heterocycle can be replaced with —NH—, —O—, or —S(O)ₐ—;

R³⁶ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; or C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂;

R³⁷ are independently C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂;

R⁴⁶ is piperazinyl optionally substituted with 1 or 2 C₁-C₆alkyl; —O(C₂-C₃alkylene)N(C₁-C₆alkyl)(C₁-C₆alkyl); or —(C₁-C₃alkylene)N(C₁-C₆alkyl)(C₁-C₆alkyl); and

R⁴⁷ is piperazinyl optionally substituted with 1 or 2 C₁-C₆alkyl; or —N(C₁-C₃alkyl)-C₂-C₃alkylene-N(C₁-C₆alkyl)(C₁-C₆alkyl).

The invention also includes pharmaceutical compositions comprising an effective amount of a compound of the present invention and a pharmaceutically acceptable carrier. The invention includes a compound of this invention when provided as a pharmaceutically acceptable prodrug, hydrated salt, such as a pharmaceutically acceptable salt, or mixtures thereof.

Representative “pharmaceutically acceptable salts” include but are not limited to, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (4,4′-methylenebis-3-hydroxy-2-naphthoate, or embonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts.

An “effective amount” when used in connection with a compound of this invention is an amount effective for inhibiting mTOR or PI3K in a subject.

The following abbreviations are used herein and have the indicated definitions: ACN is acetonitrile, AcOH is acetic acid, and ATP is adenosine triphosphate. Celite™ is flux-calcined diatomaceous earth. Celite™ is a registered trademark of World Minerals Inc. CHAPS is 3-[(3-Cholamidopropyl)dimethylammonio]-propanesulfonic acid, DEAD is diethyl azodicarboxylate, DIAD is diisopropylazodicarboxylate, DMAP is dimethyl aminopyridine, DMF is N,N-dimethylformamide, DMF-DMA is dimethylformamide dimethyl acetal, DMSO is dimethylsulfoxide, DPBS is Dulbecco's Phosphate Buffered Saline Formulation, EDTA is ethylenediaminetetraacetic acid, ESI stands for Electrospray Ionization, EtOAc is ethyl acetate, EtOH is ethanol, HEPES is 4-(2-hydroxyethyl)-1-piperazineethanesulfonic acid, GMF is Glass, Hunig's Base is diisopropylethylamine, HPLC is high pressure liquid chromatography, LPS is lipopolysaccharide, MeCN is acetonitrile, MeOH is methanol, MS is mass spectrometry, NEt₃ is triethylamine, NMR is nuclear magnetic resonance, PBS is phosphate-buffered saline (pH 7.4). Ni(Ra) is Raney™ nickel, a sponge-metal catalyst produced when a block of nickel-aluminum alloy is treated with concentrated sodium hydroxide. Raney™ is a registered trademark of W. R. Grace and Company. RPMI 1640 is a buffer (Sigma-Aldrich Corp., St. Louis, Mo., USA), SDS is dodecyl sulfate (sodium salt), SRB is Sulforhodamine B, TCA is tricholoroacetic acid, TFA is trifluoroacetic acid, THF is tetrahydrofuran, TLC is thin-layer chromatography, and TRIS is tris(hydroxymethyl)aminomethane.

In one aspect the compounds have the Formula 1, below:

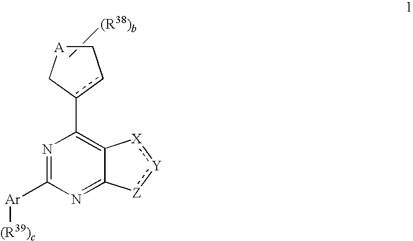

and pharmaceutically acceptable salts thereof;

wherein: R³⁸, R³⁹, the dashed lines - - - - -, Ar, X, Y, Z, b and c are as defined above for the compounds of Formula 1.

In one aspect the pyrrolopyrimidine compounds have the Formula 2, below:

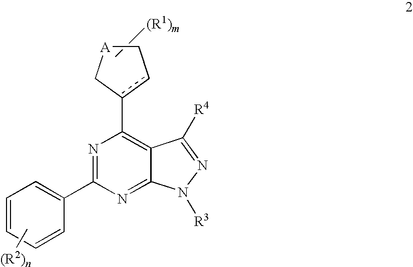

and pharmaceutically acceptable salts thereof;

wherein: R¹, R², R³, R⁴, m and n are as defined above for the compounds of Formula 2.

In one aspect, m is 0.

In one aspect, n is 1.

In one aspect, A is —CH₂—O—.

In one aspect, the dashed line - - - - - represents a second carbon-to-carbon bond.

In one aspect, R² is —NHC(O)NR⁵R⁶.

In one aspect, R⁵ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂.

In one aspect, R⁵ is methyl.

In one aspect, R⁵ is 1-fluoroethyl.

In one aspect, R⁵ is phenyl.

In one aspect, R⁵ is 3-pyridyl.

In one aspect, R⁶ is H.

In one aspect, R³ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂.

In one aspect, R³ is 1,1,1,-trifluoroethyl.

In one aspect, R⁴ is H.

In one aspect, m is 0, n is 1, A is —CH₂—O—, the dashed line - - - - - represents a second carbon to carbon bond, R² is —NHC(O)NR⁵R⁶, R³ is 1,1,1,-trifluoroethyl, and R⁴ is H.

In one aspect, m is 0, n is 1, A is —CH₂—O—, the dashed line - - - - - represents a second carbon to carbon bond, R² is —NHC(O)NR⁵R⁶, R⁵ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂, R⁶ is H, R³ is 1,1,1,-trifluoroethyl, and R⁴ is H.

In one aspect, m is 0; n is 1; A is —CH₂—O—; the dashed line - - - - - represents a second carbon to carbon bond; R² is —NHC(O)NR⁵R⁶; R⁵ is selected from the group consisting of methyl, 1-fluoroethyl, phenyl, and 3-pyridyl; R⁶ is H; R³ is 1,1,1,-trifluoroethyl; and R⁴ is H.

Illustrative compounds of Formula 2 are exemplified by the following compounds:
3-[4-(3,6-dihydro-2H-pyran-4-yl)-1-phenyl-1H-pyrazolo[3,4-d]pyrimidin-6-yl]phenol;
3-[1-phenyl-4-(tetrahydro-2H-pyran-4-yl)-1H-pyrazolo[3,4-d]pyrimidin-6-yl]phenol;
N-{4-[1-(1-benzylpiperidin-4-yl)-4-(3,6-dihydro-2H-pyran-4-yl)-1H-pyrazolo[3,4-d]pyrimidin-6-yl]phenyl}acetamide;
1-(4-{4-(3,6-dihydro-2H-pyran-4-yl)-1-[1-(pyridin-3-ylmethyl)piperidin-4-yl]-1H-pyrazolo[3,4-d]pyrimidin-6-yl}phenyl)-3-methylurea;
1-(4-{4-(3,6-dihydro-2H-pyran-4-yl)-1-[1-(pyridin-3-ylcarbonyl)piperidin-4-yl]-1H-pyrazolo[3,4-d]pyrimidin-6-yl}phenyl)-3-methylurea;
1-{4-[4-(3,6-dihydro-2H-pyran-4-yl)-1-(2,2,2-trifluoroethyl)-1H-pyrazolo[3,4-d]pyrimidin-6-yl]phenyl}-3-methylurea;
1-{4-[4-(3,6-dihydro-2H-pyran-4-yl)-1-(2,2,2-trifluoroethyl)-1H-pyrazolo[3,4-d]pyrimidin-6-yl]phenyl}-3-(2-fluoroethyl)urea;
1-{4-[4-(3,6-dihydro-2H-pyran-4-yl)-1-(2,2,2-trifluoroethyl)-1H-pyrazolo[3,4-d]pyrimidin-6-yl]phenyl}-3-pyridin-3-ylurea;
1-{4-[4-(3,6-dihydro-2H-pyran-4-yl)-1-(2,2,2-trifluoroethyl)-1H-pyrazolo[3,4-d]pyrimidin-6-yl]phenyl}-3-phenylurea;
1-{4-[4-(3,6-dihydro-2H-pyran-4-yl)-1-(2,2,2-trifluoroethyl)-1H-pyrazolo[3,4-d]pyrimidin-6-yl]phenyl}-3-ethylurea;
1-{4-[4-(3,6-dihydro-2H-pyran-4-yl)-1-(2,2,2-trifluoroethyl)-1H-pyrazolo[3,4-d]pyrimidin-6-yl]phenyl}-3-(4-(4-methylpiperazin-1-yl)phenyl)urea;
1-{4-[4-(3,6-dihydro-2H-pyran-4-yl)-1-(2,2,2-trifluoroethyl)-1H-pyrazolo[3,4-d]pyrimidin-6-yl]phenyl}-3-(4-(4-methylpiperazine-1-carbonyl)phenyl)urea;
2-hydroxyethyl{4-[4-(3,6-dihydro-2H-pyran-4-yl)-1-(2,2,2-trifluoroethyl)-1H-pyrazolo[3,4-d]pyrimidin-6-yl]phenyl}carbamate;
2-hydroxyethyl{4-[4-(tetrahydro-2H-pyran-4-yl)-1-(2,2,2-trifluoroethyl)-1H-pyrazolo[3,4-d]pyrimidin-6-yl]phenyl}carbamate; and
1-(pyridin-3-yl)-3-(4-(4-(tetrahydro-2H-pyran-4-yl)-1-(2,2,2-trifluoroethyl)-1H-pyrazolo[3,4-d]pyrimidin-6-yl)phenyl)urea.

In one aspect the purine compounds have the Formula 3, below:

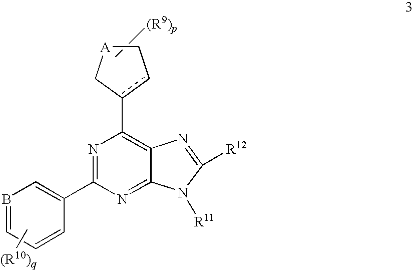

tautomers thereof and pharmaceutically acceptable salts thereof;

wherein: B, R⁹, R¹⁰, R¹¹, R¹², p and q are as defined above for the compounds of Formula 3.

In one aspect, p is 0.

In one aspect, q is 1.

In one aspect, A is —CH₂—O—.

In one aspect, the dashed line - - - - - represents a second carbon-to-carbon bond.

In one aspect, R¹⁰ is —NHC(O)NR¹³R¹⁴.

In one aspect, R¹³ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂.

In one aspect, R¹³ is methyl.

In one aspect, R¹³ is 1-fluoroethyl.

In one aspect, R¹³ is phenyl.

In one aspect, R¹³ is 3-pyridyl.

In one aspect, R¹⁴ is H.

In one aspect, R¹¹ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂.

In one aspect, R¹¹ is 1,1,1,-trifluoroethyl.

In one aspect, R¹¹ is 4- to 7-membered monocyclic heterocycle group which is unsubstituted or is substituted with from 1 to 3 substituents independently selected from C₁-C₈acyl, C₁-C₆alkyl, heterocyclylalkyl, wherein the ring portion of the heterocyclylalkyl group is unsubstituted or is substituted by 1 to 3 substituents independently selected from halogen, —NH₂, —O(C₁-C₆alkyl), C₁-C₆alkyl, 4- to 7-membered monocyclic heterocycle, and C₃-C₈cycloalkyl, (C₆-C₁₄aryl)alkyl, wherein the ring portion of the (C₆-C₁₄aryl)alkyl group is unsubstituted or is substituted by 1 to 3 substituents independently selected from halogen, —NH₂, —O(C₁-C₆alkyl), C₁-C₆alkyl, 4- to 7-membered monocyclic heterocycle, and C₃-C₈cycloalkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂.

In one aspect, R¹² is H.

In one aspect, p is 0, q is 1, A is —CH₂—O—, the dashed line - - - - - represents a second carbon to carbon bond, R¹⁰ is —NHC(O)NR¹³R¹⁴, R¹¹ is 1,1,1,-trifluoroethyl, and R¹² is H.

In one aspect, p is 0, q is 1, A is —CH₂—O—, the dashed line - - - - - represents a second carbon to carbon bond, R¹⁰ is —NHC(O)NR¹³R¹⁴, R¹³ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂, R¹⁴ is H, R¹¹ is 1,1,1,-trifluoroethyl, and R¹² is H.

In one aspect, p is 0; q is 1; A is —CH₂—O—; the dashed line - - - - - represents a second carbon to carbon bond; R¹⁰ is —NHC(O)NR¹³R¹⁴; R¹³ is selected from the group consisting of methyl, 1-fluoroethyl, phenyl, and 3-pyridyl; R¹⁴ is H; R¹¹ is 1,1,1,-trifluoroethyl; and R¹² is H.

Illustrative compounds of Formula 3 are exemplified by the following compounds:
3-[6-(3,6-dihydro-2H-pyran-4-yl)-9H-purin-2-yl]phenol;
3-[6-(3,6-dihydro-2H-pyran-4-yl)-9-piperidin-4-yl-9H-purin-2-yl]phenol;
3-[9-(1-benzylpiperidin-4-yl)-6-(3,6-dihydro-2H-pyran-4-yl)-9H-purin-2-yl]phenol;
3-{6-(3,6-dihydro-2H-pyran-4-yl)-9-[1-(2-furylmethyl)piperidin-4-yl]-9H-purin-2-yl}phenol;
3-[6-(3,6-dihydro-2H-pyran-4-yl)-9-{1-[(6-morpholin-4-ylpyridin-3-yl)methyl]piperidin-4-yl}-9H-purin-2-yl]phenol;
3-{6-(3,6-dihydro-2H-pyran-4-yl)-9-[1-(1H-pyrrol-2-ylmethyl)piperidin-4-yl]-9H-purin-2-yl}phenol;
3-[6-(3,6-dihydro-2H-pyran-4-yl)-9-(1-methylpiperidin-4-yl)-9H-purin-2-yl]phenol;
3-{6-(3,6-dihydro-2H-pyran-4-yl)-9-[1-(1H-imidazol-5-ylmethyl)piperidin-4-yl]-9H-purin-2-yl}phenol;
3-{6-(3,6-dihydro-2H-pyran-4-yl)-9-[1-(4-methylbenzyl)piperidin-4-yl]-9H-purin-2-yl}phenol;
3-[9-{1-[(6-bromopyridin-3-yl)methyl]piperidin-4-yl}-6-(3,6-dihydro-2H-pyran-4-yl)-9H-purin-2-yl]phenol;
3-{9-[1-(3,4-difluorobenzyl)piperidin-4-yl]-6-(3,6-dihydro-2H-pyran-4-yl)-9H-purin-2-yl}phenol;
{3-[9-(1-benzylpiperidin-4-yl)-6-(3,6-dihydro-2H-pyran-4-yl)-9H-purin-2-yl]phenyl}methanol;
{3-[6-(3,6-dihydro-2H-pyran-4-yl)-9-{1-[(6-fluoropyridin-3-yl)methyl]piperidin-4-yl}-9H-purin-2-yl]phenyl}methanol;
(3-{6-(3,6-dihydro-2H-pyran-4-yl)-9-[1-(pyridin-3-ylmethyl)piperidin-4-yl]-9H-purin-2-yl}phenyl)methanol;
(3-{6-(3,6-dihydro-2H-pyran-4-yl)-9-[1-(pyridin-2-ylmethyl)piperidin-4-yl]-9H-purin-2-yl}phenyl)methanol;
{3-[9-{1-[(6-bromopyridin-3-yl)methyl]piperidin-4-yl}-6-(3,6-dihydro-2H-pyran-4-yl)-9H-purin-2-yl]phenyl}methanol;
tert-butyl 4-{6-(3,6-dihydro-2H-pyran-4-yl)-2-[3-(hydroxymethyl)phenyl]-9H-purin-9-yl}piperidine-1-carboxylate;
{3-[6-(3,6-dihydro-2H-pyran-4-yl)-9-piperidin-4-yl-9H-purin-2-yl]phenyl}methanol;
1-(4-{6-(3,6-dihydro-2H-pyran-4-yl)-9-[1-(1H-pyrrol-2-ylmethyl)piperidin-4-yl]-9H-purin-2-yl}phenyl)-3-methylurea;
1-{4-[6-(3,6-dihydro-2H-pyran-4-yl)-9-piperidin-4-yl-9H-purin-2-yl]phenyl}-3-piperidin-4-ylurea;
benzyl 4-{[(4-{6-(3,6-dihydro-2H-pyran-4-yl)-9-[1-(1H-pyrrol-2-ylmethyl)piperidin-4-yl]-9H-purin-2-yl}phenyl)carbamoyl]amino}piperidine-1-carboxylate;
5-[6-(3,6-dihydro-2H-pyran-4-yl)-9-piperidin-4-yl-9H-purin-2-yl]pyridin-3-ol;
{3-[6-(3,6-dihydro-2H-pyran-4-yl)-9-(2-piperidin-1-ylethyl)-9H-purin-2-yl]phenyl}methanol;
5-[9-(1-benzylpiperidin-4-yl)-6-(3,6-dihydro-2H-pyran-4-yl)-9H-purin-2-yl]pyridin-3-ol;
5-{6-(3,6-dihydro-2H-pyran-4-yl)-9-[1-(pyridin-3-ylmethyl)piperidin-4-yl]-9H-purin-2-yl}pyridin-3-ol;
5-[9-{1-[(6-bromopyridin-3-yl)methyl]piperidin-4-yl}-6-(3,6-dihydro-2H-pyran-4-yl)-9H-purin-2-yl]pyridin-3-ol;
5-{6-(3,6-dihydro-2H-pyran-4-yl)-9-[1-(pyridin-2-ylmethyl)piperidin-4-yl]-9H-purin-2-yl}pyridin-3-ol;
3-[6-(3,6-dihydro-2H-pyran-4-yl)-9-(2-piperidin-1-ylethyl)-9H-purin-2-yl]phenol;
5-[6-(3,6-dihydro-2H-pyran-4-yl)-9-{1-[(6-methoxypyridin-3-yl)methyl]piperidin-4-yl}-9H-purin-2-yl]pyridin-3-ol;
5-[6-(3,6-dihydro-2H-pyran-4-yl)-9-{1-[(5-fluoro-1H-indol-3-yl)methyl]piperidin-4-yl}-9H-purin-2-yl]pyridin-3-ol;
5-[9-{1-[(2-aminopyridin-3-yl)methyl]piperidin-4-yl}-6-(3,6-dihydro-2H-pyran-4-yl)-9H-purin-2-yl]pyridin-3-ol;
5-[9-{1-[(5-bromopyridin-3-yl)methyl]piperidin-4-yl}-6-(3,6-dihydro-2H-pyran-4-yl)-9H-purin-2-yl]pyridin-3-ol;
5-[6-(3,6-dihydro-2H-pyran-4-yl)-9-{1-[(2-methoxypyridin-3-yl)methyl]piperidin-4-yl}-9H-purin-2-yl]pyridin-3-ol;
5-[9-{1-[(6-chloropyridin-3-yl)methyl]piperidin-4-yl}-6-(3,6-dihydro-2H-pyran-4-yl)-9H-purin-2-yl]pyridin-3-ol;
6-(3,6-dihydro-2H-pyran-4-yl)-2-(3-hydroxyphenyl)-7,9-dihydro-8H-purin-8-one;
6-(3,6-dihydro-2H-pyran-4-yl)-2-[3-(hydroxymethyl)phenyl]-7,9-dihydro-8H-purin-8-one;
1-(4-(6-(3,6-dihydro-2H-pyran-4-yl)-9-ethyl-9H-purin-2-yl)phenyl)-3-(4-(4-methylpiperazin-1-yl)phenyl)urea;
1-(4-(6-(3,6-dihydro-2H-pyran-4-yl)-9-ethyl-9H-purin-2-yl)phenyl)-3-(pyridin-4-yl)urea; and
1-(4-(6-(3,6-dihydro-2H-pyran-4-yl)-9-ethyl-9H-purin-2-yl)phenyl)-3-(4-(4-methylpiperazine-1-carbonyl)phenyl)urea.

In one aspect the 3H-[1,2,3]triazolo[4,5-d]pyrimidine compounds have the Formula 4, below:

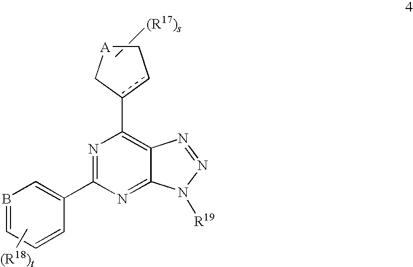

and pharmaceutically acceptable salts thereof;

wherein: R¹⁷, R¹⁸, R¹⁹, B, s and t are as defined above for the compounds of Formula 4.

In one aspect, s is 0.

In one aspect, t is 1.

In one aspect, A is —CH₂—O—.

In one aspect, the dashed line - - - - - represents a second carbon-to-carbon bond.

In one aspect, R¹⁸ is —NHC(O)NR²⁰R²¹.

In one aspect, R²⁰ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂.

In one aspect, R²⁰ is methyl.

In one aspect, R²⁰ is 1-fluoroethyl.

In one aspect, R²⁰ is phenyl.

In one aspect, R²⁰ is 3-pyridyl.

In one aspect, R²¹ is H.

In one aspect, R¹⁹ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂.

In one aspect, R¹⁹ is 1,1,1,-trifluoroethyl.

In one aspect, B is CH.

In one aspect, s is 0, t is 1, A is —CH₂—O—, the dashed line - - - - - represents a second carbon to carbon bond, R¹⁸ is —NHC(O)NR²⁰R²¹, R¹⁹ is 1,1,1,-trifluoroethyl, and B is CH.

In one aspect, s is 0, t is 1, A is —CH₂—O—, the dashed line - - - - - represents a second carbon to carbon bond, R¹⁸ is —NHC(O)NR²⁰R²¹, R²⁰ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂, R²¹ is H, R¹⁹ is 1,1,1,-trifluoroethyl, and B is CH.

In one aspect, s is 0; t is 1; A is —CH₂—O—; the dashed line - - - - - represents a second carbon to carbon bond; R¹⁸ is —NHC(O)NR²⁰R²¹; R²⁰ is selected from the group consisting of methyl, 1-fluoroethyl, phenyl, and 3-pyridyl; R²¹ is H; R¹⁹ is 1,1,1,-trifluoroethyl; and B is CH.

Illustrative compounds of Formula 4 are exemplified by the following compounds:
3-[7-(3,6-dihydro-2H-pyran-4-yl)-3H-[1,2,3]triazolo[4,5-d]pyrimidin-5-yl]phenol;
{3-[7-(3,6-dihydro-2H-pyran-4-yl)-3H-[1,2,3]triazolo[4,5-d]pyrimidin-5-yl]phenyl}methanol;
5-[7-(3,6-dihydro-2H-pyran-4-yl)-3H-[1,2,3]triazolo[4,5-d]pyrimidin-5-yl]pyridin-3-ol;
3-[3-(1-benzylpiperidin-4-yl)-7-(3,6-dihydro-2H-pyran-4-yl)-3H-[1,2,3]triazolo[4,5-d]pyrimidin-5-yl]phenol;
4-(3-(4-(7-(3,6-dihydro-2H-pyran-4-yl)-3-ethyl-3H-[1,2,3]triazolo[4,5-d]pyrimidin-5-yl)phenyl)ureido)-N-(2-(dimethylamino)ethyl)-N-methylbenzamide;
1-(4-(7-(3,6-dihydro-2H-pyran-4-yl)-3-ethyl-3H-[1,2,3]triazolo[4,5-d]pyrimidin-5-yl)phenyl)-3-(4-(4-methylpiperazine-1-carbonyl)phenyl)urea;
1-(4-(7-(3,6-dihydro-2H-pyran-4-yl)-3-ethyl-3H-[1,2,3]triazolo[4,5-d]pyrimidin-5-yl)phenyl)-3-(4-(4-methylpiperazin-1-yl)phenyl)urea; and
1-(4-(7-(3,6-dihydro-2H-pyran-4-yl)-3-ethyl-3H-[1,2,3]triazolo[4,5-d]pyrimidin-5-yl)phenyl)-3-(pyridin-4-yl)urea.

In one aspect the thieno[2,3-d]pyrimidine compounds have the Formula (5), below:

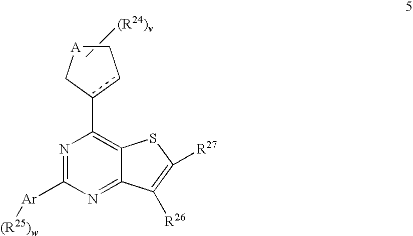

and pharmaceutically acceptable salts thereof;

wherein: R²⁴, R²⁵, R²⁶, R²⁷, Ar, v and w are as defined above for the compounds of Formula 5.

In one aspect, v is 0.

In one aspect, w is 1.

In one aspect, A is —CH₂—O—.

In one aspect, the dashed line - - - - - represents a second carbon-to-carbon bond.

In one aspect, R²⁵ is —NHC(O)NR²⁸R²⁹.

In one aspect, R²⁸ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂.

In one aspect, R²⁸ is methyl.

In one aspect, R²⁸ is 1-fluoroethyl.

In one aspect, R²⁸ is phenyl.

In one aspect, R²⁸ is 3-pyridyl.

In one aspect, R²⁹ is H.

In one aspect, Ar is phenyl.

In one aspect, R²⁶ is H.

In one aspect, R²⁷ is H.

In one aspect, v is 0, w is 1, A is —CH₂—O—, the dashed line - - - - - represents a second carbon to carbon bond, R²⁵ is —NHC(O)NR²⁸R²⁹, Ar is phenyl, and R²⁶ is H.

In one aspect, v is 0, w is 1, A is —CH₂—O—, the dashed line - - - - - represents a second carbon to carbon bond, R²⁵ is —NHC(O)NR²⁸R²⁹, R²⁸ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂, R²⁹ is H, Ar is phenyl, and R²⁶ is H.

In one aspect, v is 0; w is 1; A is —CH₂—O—; the dashed line - - - - - represents a second carbon to carbon bond; R²⁵ is —NHC(O)NR²⁸R²⁹; R²⁸ is selected from the group consisting of methyl, 1-fluoroethyl, phenyl, and 3-pyridyl; R⁶ is H; Ar is phenyl; and R²⁶ is H.

Illustrative compounds of Formula 5 are exemplified by the following compounds:
1-(4-(4-(3,6-dihydro-2H-pyran-4-yl)thieno[2,3-d]pyrimidin-2-yl)phenyl)-3-ethylurea;
1-(4-(4-(3,6-dihydro-2H-pyran-4-yl)thieno[2,3-d]pyrimidin-2-yl)phenyl)-3-(2-fluoroethyl)urea;
1-(4-(4-(3,6-dihydro-2H-pyran-4-yl)thieno[2,3-d]pyrimidin-2-yl)phenyl)-3-phenylurea;
1-(4-(4-(3,6-dihydro-2H-pyran-4-yl)thieno[2,3-d]pyrimidin-2-yl)phenyl)-3-(pyridin-3-yl)urea;
1-(4-(4-(3,6-dihydro-2H-pyran-4-yl)thieno[2,3-d]pyrimidin-2-yl)phenyl)-3-(pyridin-4-yl)urea;
4-(4-(3,6-dihydro-2H-pyran-4-yl)thieno[2,3-d]pyrimidin-2-yl)aniline;
N-(4-(4-(3,6-dihydro-2H-pyran-4-yl)thieno[2,3-d]pyrimidin-2-yl)phenyl)acetamide;
methyl 4-(4-(3,6-dihydro-2H-pyran-4-yl)thieno[2,3-d]pyrimidin-2-yl)phenylcarbamate;
3-(4-(3,6-dihydro-2H-pyran-4-yl)thieno[2,3-d]pyrimidin-2-yl)phenol;
4-(4-(3,6-dihydro-2H-pyran-4-yl)thieno[2,3-d]pyrimidin-2-yl)phenol;
4-(3,6-dihydro-2H-pyran-4-yl)-2-(1H-indol-5-yl)thieno[2,3-d]pyrimidine;
5-(4-(3,6-dihydro-2H-pyran-4-yl)thieno[2,3-d]pyrimidin-2-yl)pyridin-2-amine;
2-hydroxyethyl 4-(4-(3,6-dihydro-2H-pyran-4-yl)thieno[3,2-d]pyrimidin-2-yl)phenylcarbamate;
1-(4-(4-(3,6-dihydro-2H-pyran-4-yl)thieno[3,2-d]pyrimidin-2-yl)phenyl)-3-(4-(4-methylpiperazin-1-yl)phenyl)urea; and
1-(pyridin-3-yl)-3-(4-(4-(tetrahydro-2H-pyran-4-yl)thieno[3,2-d]pyrimidin-2-yl)phenyl)urea.

In one aspect the purine compounds have the Formula 6, below:

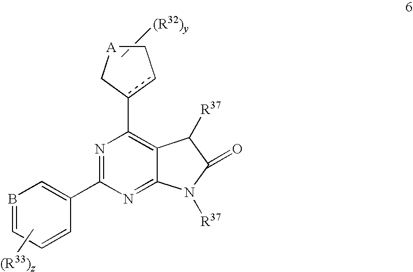

and pharmaceutically acceptable salts thereof;

wherein: R³², R³³, R³⁷, B, y and z are as defined above for the compounds of Formula 6.

In one aspect, y is 0.

In one aspect, z is 1.

In one aspect, A is —CH₂—O—.

In one aspect, the dashed line - - - - - represents a second carbon-to-carbon bond.

In one aspect, R³³ is —NHC(O)NR³⁴R³⁵.

In one aspect, R³⁴ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂.

In one aspect, R³⁴ is methyl.

In one aspect, R³⁴ is 1-fluoroethyl.

In one aspect, R³⁴ is phenyl.

In one aspect, R³⁴ is 3-pyridyl.

In one aspect, R³⁵ is H.

In one aspect, R³⁷ is C₁-C₆alkyl, which is unsubstituted or is substituted with from 1 to 3 substituents independently selected from halogen and C₆-C₁₄aryl.

In one aspect, R³⁷ both are 1,1,1,-trifluoroethyl.

In one aspect, R³⁷ both are benzyl.

In one aspect, y is 0, z is 1, A is —CH₂—O—, the dashed line - - - - - represents a second carbon to carbon bond, R³³ is —NHC(O)NR³⁴R³⁵, R³⁷ both are 1,1,1,-trifluoroethyl, and B is CH.

In one aspect, y is 0, z is 1, A is —CH₂—O—, the dashed line - - - - - represents a second carbon to carbon bond, R³³ is —NHC(O)NR³⁴R³⁵, R³⁴ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂, R³⁵ is H, R⁷ both are 1,1,1,-trifluoroethyl, and B is CH.

In one aspect, y is 0; z is 1; A is —CH₂—O—; the dashed line - - - - - represents a second carbon to carbon bond; R³³ is —NHC(O)NR³⁴R³⁵; R³⁴ is selected from the group consisting of methyl, 1-fluoroethyl, phenyl, and 3-pyridyl; R³⁵ is H; R³⁷ both are 1,1,1,-trifluoroethyl; and B is CH.

Illustrative compounds of Formula 6 are exemplified by the following compound: 7,9-dibenzyl-6-(3,6-dihydro-2H-pyran-4-yl)-2-(3-hydroxyphenyl)-7,9-dihydro-8H-purin-8-one.

In another aspect, the invention provides methods of synthesizing compounds of the Formula 2 comprising: a) reacting a hydrazine of the formula H₂N—NH—R³ with the nitrile 8:

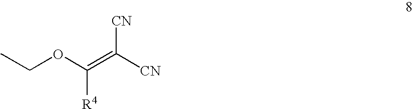

wherein R³ and R⁴, are as defined in Formula 2 to give the aminopyrazole 9:

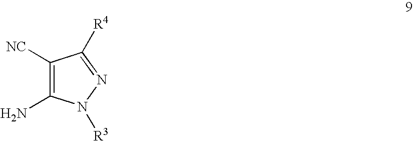

(b) reacting the amino pyrazole of Formula 9 with an acid chloride compound 10 to acylate the amino group at position 3 of the pyrazole, wherein R², R⁵, R⁶, R⁷, R⁸, n, and o are as defined in Formula 2

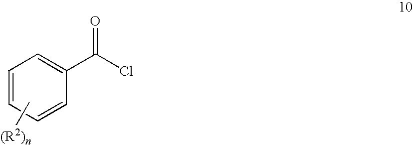

under conditions effective to acylate the amino group at position 3 of the pyrazole ring thereby producing amide 11;

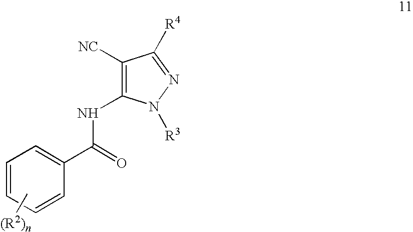

(c) cyclizing the amide 11 under oxidizing conditions and chlorinating the newly formed lactam to introduce a chlorine atom at position 4 of the 1H-pyrazolo[3,4-d]pyrimidine thereby producing the chlorinated intermediate 12;

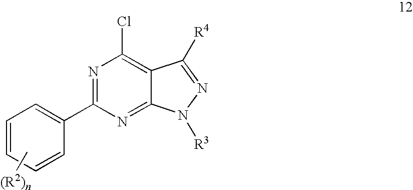

(d) reacting the chloro 1H-pyrazolo[3,4-d]pyrimidine of Formula 12 with a tributylstannane compound 13 to substitute the chlorine atom at position 4 of the 1H-pyrazolo[3,4-d]pyrimidine compound 12 with the organic moiety from the stannane 13:

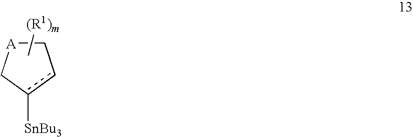

wherein A, the dashed line - - - - -, R¹, and m are as defined in Formula 2; under conditions effective to substitute the chlorine atom at position 4 of the 1H-pyrazolo[3,4-d]pyrimidine thereby providing a compound of the Formula 2;

(e) optionally reducing the double bond of the 1H-pyrazolo[3,4-d]pyrimidine of Formula 2, thereby producing the 1H-pyrazolo[3,4-d]pyrimidine of Formula 14:

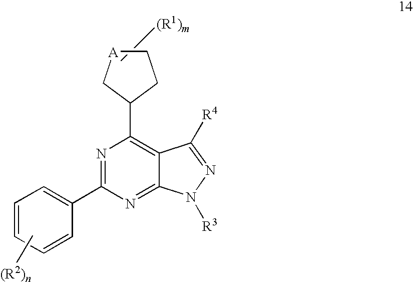

or a pharmaceutically acceptable salt thereof.

In another aspect, the invention provides methods of synthesizing compounds of the Formula 3 comprising: a) reacting a 2,4-dichloropurine of the Formula 15 with the alcohol R¹¹OH under Mitsunobu conditions:

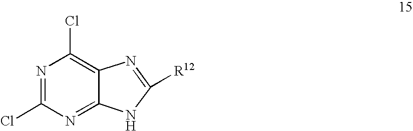

wherein R¹¹ and R¹² are as defined in Formula 3 under conditions effective to substitute the nitrogen atom at position 9 of the 9H-purine thereby providing a compound of the Formula 16:

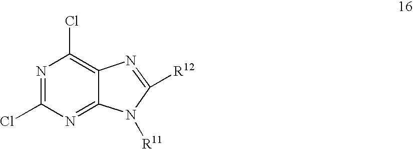

(b) reacting the dichloro purine of Formula 16 with a tributylstannane compound 17:

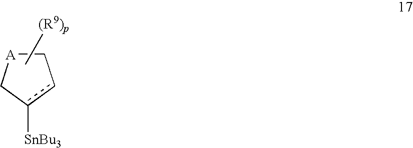

under conditions effective to replace the chlorine atom at position 6 of the purine ring, thereby providing a compound of the Formula 18:

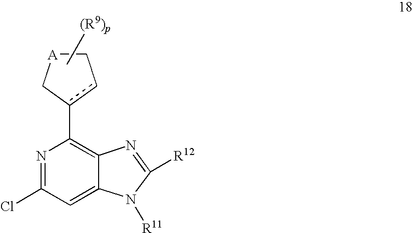

wherein R⁹, and p are as defined in Formula 3 under conditions effective to replace the chlorine atom at position 6 of the purine ring;

c) performing a Suzuki coupling on the chloropurine 18 with the boronic acid 19

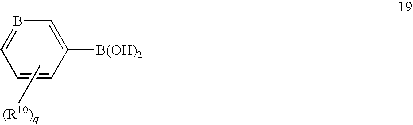

wherein the B in the aromatic ring, R¹⁰, and q are as defined in Formula 3, under conditions effective to substitute the chlorine atom at position 2 of the purine ring with the aromatic radical from the boronic acid thereby providing a compound of the Formula 3, a tautomer thereof, or a pharmaceutically acceptable salt thereof.

In another aspect, the invention provides methods of synthesizing compounds of the Formula 4 comprising: a) reacting 5-nitro-2,4,6-trichloropyrimidine of the Formula 20 with the amine R¹⁹NH₂;

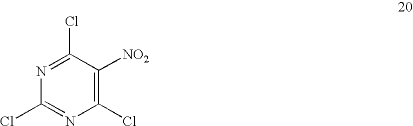

wherein R¹⁹ is as defined in Formula 4 under conditions effective to displace the chlorine atom at position 6 of the pyrimidine ring to give the dichloropyrimidine intermediate of Formula 21:

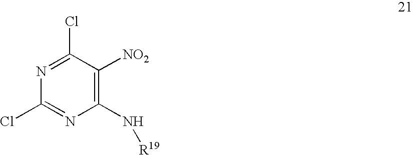

(b) reacting the dichloro compound of Formula 21 with a tributylstannane compound 22 to substitute the chlorine atom at position 4 of the pyrimidine compound 21 with the organic moiety from the tributylstannane 22 under conditions effective to replace the chlorine atom thereby providing a compound of the Formula 23:

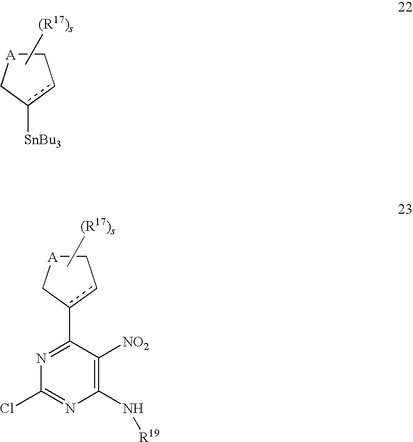

wherein R¹⁷ and s are as defined in Formula 4;

c) reducing the compound of Formula 23 under conditions effective to reduce the nitro group at position 5 of the pyrimidine ring to an amino group without reducing the organic moiety at position 4 of the pyrimidine ring thereby providing a monochloro compound of the Formula 24:

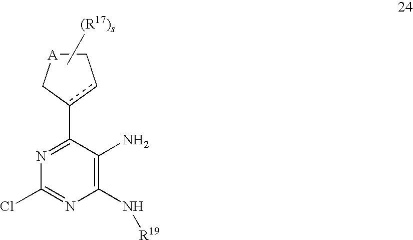

(d) performing either a two step sequence of Suzuki coupling with the boronic acid 25

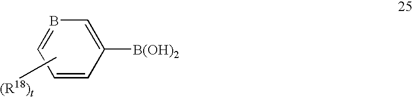

followed by diazotization and cyclization, or a two step sequence of diazotization and cyclization followed by Suzuki coupling with the boronic acid 25, wherein the B in the aromatic ring, R¹⁸, and t are as defined in Formula 4, under conditions effective to substitute the chlorine atom at position 2 of the pyrimidine ring with the aromatic radical from the boronic acid thereby providing a compound of the Formula 4 or a pharmaceutically acceptable salt thereof.

In another aspect, the invention provides methods of synthesizing compounds of the Formula 5 comprising: a) reacting a 2-amido-3-amino-thiophene of the Formula 26:

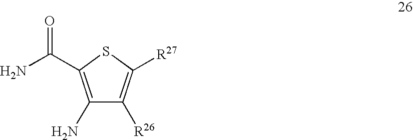

wherein R²⁶ and R²⁷ are as defined in Formula 5 with triphosgene under conditions effective to cyclized the fused pyrimidine ring to give the thieno[3,2-d]pyrimidine intermediate of Formula 27:

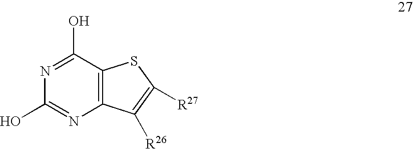

b) reacting the compound of Formula 27 with phosphorous oxychloride under conditions effective to substitute the hydroxyl groups at positions 2 and 4 of thieno[3,2-d]pyrimidine 27 with chlorine atoms from the phosphorus oxychloride thereby providing a dichloro compound of the Formula 28:

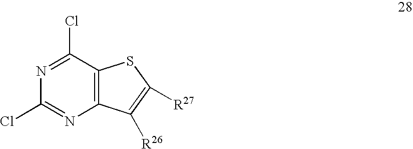

(c) reacting the dichloro compound of Formula 28 with a tributylstannane compound 29 to substitute the chlorine atom at position 4 of the thieno[3,2-d]pyrimidine compound 28 with the organic moiety from the tributylstannane under conditions effective to replace the chlorine atom thereby providing a compound of the Formula 30:

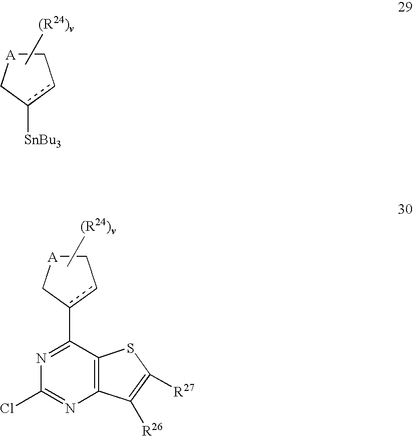

wherein A, the dashed line - - - - -, R²⁴, and v are as defined in Formula 5;

(d) reacting the compound of Formula 30 with a boronic acid of the structure:

(R²⁵)_{w}—ArB(OH)₂

wherein Ar, R²⁵, w, x, R²⁸, R²⁹, and R³⁰ are as defined in Formula 5, under conditions effective to substitute the chlorine atom at position 2 of the thieno[3,2-d]pyrimidine ring with the R_{w}²⁵—Ar radical from the boronic acid thereby providing a compound of the Formula 5.

In another aspect, the invention provides methods of synthesizing compounds of the Formula 6, or a pharmaceutically acceptable salt thereof, comprising: reacting the compound of Formula 31 at the two imidazole

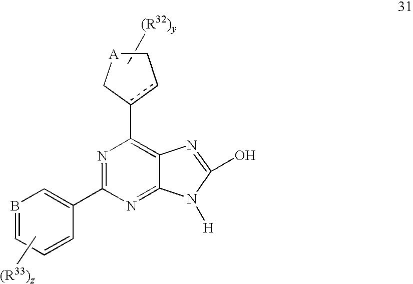

nitrogen atoms by treating with a base and an alkylating agent R³⁷X under conditions effective to substitute the hydrogen atoms at positions 7 and 9 of purine compound 31 with the R³⁷ group from the alkylating agent thereby providing a compound of the Formula 6 wherein A, the dashed line - - - - -, R³², y, B, R³³, z, a, R³⁴, R³⁵, R³⁶, and R³⁷ as defined in Formula 6 and X is halogen.

### DEFINITIONS

The following definitions are used in connection with the compounds of the present invention unless the context indicates otherwise:

“Acyl” refers to from 1 to 8 carbon atoms of a straight, branched, or cyclic configuration or a combination thereof, attached to the parent structure through a carbonyl functionality. Such groups may be saturated or unsaturated, aliphatic or aromatic, and carbocyclic or heterocyclic. One or more carbons in the acyl residue may be replaced by oxygen, nitrogen (e.g., carboxyamido), or sulfur as long as the point of attachment to the parent remains at the carbonyl. Examples of a C₁-C₈acyl group include acetyl-, benzoyl-, nicotinoyl, propionyl-, isobutyryl-, oxalyl-, t-butoxycarbonyl-, benzyloxycarbonyl, morpholinylcarbonyl, and the like. Lower-acyl refers to acyl groups containing one to four carbons. An acyl group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl.

“Alkenyl” refers to a straight or branched chain unsaturated hydrocarbon containing 2-10 carbon atoms and at least one double bond. Examples of a C₂-C₁₀alkenyl group include, but are not limited to, ethylene, propylene, 1-butylene, 2-butylene, isobutylene, sec-butylene, 1-pentene, 2-pentene, isopentene, 1-hexene, 2-hexene, 3-hexene, isohexene, 1-heptene, 2-heptene, 3-heptene, 1-octene, 2-octene, 3-octene, 4-octene, 1-nonene, 2-nonene, 3-nonene, 4-nonene, 1-decene, 2-decene, 3-decene, 4-decene and 5-decene. A alkenyl group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl.

“Alkoxy” refers to the group R—O— where R is an alkyl group, as defined below. Exemplary alkoxy groups include but are not limited to methoxy, ethoxy, n-propoxy, 1-propoxy, n-butoxy and t-butoxy. An alkoxy group can be unsubstituted or substituted with one or more of the following groups: halogen, hydroxyl, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —O(C₁-C₆alkyl), —C(O)OH, —C(O)O(C₁-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), carboxyamidoalkyl-, or —NO₂;.

“Alkoxycarbonyl” refers to the group alkyl-O—C(O)—. An alkoxycarbonyl group can be unsubstituted or substituted with one or more of the following groups: halogen, hydroxyl, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —O(C₁-C₆alkyl), —C(O)OH, —C(O)O(C₁-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), carboxyamidoalkyl-, or —NO₂.

“Alkyl” refers to a hydrocarbon chain that may be a straight chain or branched chain, containing the indicated number of carbon atoms. For example, C₁-C₁₀ indicates that the group may have from 1 to 10 (inclusive) carbon atoms in it. In the absence of any numerical designation, “alkyl” is a chain (straight or branched) having 1 to 6 (inclusive) carbon atoms in it. Examples of C₁-C₆ alkyl groups include, but are not limited to, methyl, ethyl, propyl, butyl, pentyl, hexyl, isopropyl, isobutyl, sec-butyl, tert-butyl, isopentyl, neopentyl, and isohexyl. An alkyl group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), carboxyamidoalkyl-, or —NO₂.

The carbon number as used in the definitions herein refers to carbon backbone and carbon branching, but does not include carbon atoms of the substituents, such as alkoxy substitutions and the like.

“(Alkyl)amido-” refers to a —C(O)NH— group in which the nitrogen atom of said group is attached to a alkyl group, as defined above. Representative examples of a (C₁-C₆alkyl)amido group include, but are not limited to, —C(O)NHCH₃, —C(O)NHCH₂CH₃, —C(O)NHCH₂CH₂CH₃, —C(O)NHCH₂CH₂CH₂CH₃, —C(O)NHCH₂CH₂CH₂CH₂CH₃, —C(O)NHCH(CH₃)₂, C(O)NHCH₂CH(CH₃)₂, —C(O)NHCH(CH₃)CH₂CH₃, —C(O)NH—C(CH₃)₃ and —C(O)NHCH₂C(CH₃)₃.

“Alkylamino-” refers to an —NH group, the nitrogen atom of said group being attached to a alkyl group, as defined above. Representative examples of an C₁-C₆alkylamino group include, but are not limited to —NHCH₃, —NHCH₂CH₃, —NHCH₂CH₂CH₃, —NHCH₂CH₂CH₂CH₃, —NHCH(CH₃)₂, —NHCH₂CH(CH₃)₂, —NHCH(CH₃)CH₂CH₃ and —NH—C(CH₃)₃. An alkylamino group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C1]-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), carboxyamidoalkyl-, or —NO₂.

“Alkylcarboxy” refers to an alkyl group as defined above, attached to the parent structure through the oxygen atom of a carboxyl (C(O)—O—) functionality. Examples of C₁-C₆alkylcarboxy include acetoxy, ethylcarboxy, propylcarboxy, and isopentylcarboxy.

“(Alkyl)carboxyamido-” refers to a —NHC(O)— group in which the carbonyl carbon atom of said group is attached to a alkyl group, as defined above. Representative examples of a (C₁-C₆alkyl)carboxyamido group include, but are not limited to, —NHC(O)CH₃, —NHC(O)CH₂CH₃, —NHC(O)CH₂CH₂CH₃, —NHC(O)CH₂CH₂CH₂CH₃, —NHC(O)CH₂CH₂CH₂CH₂CH₃, —NHC(O)CH(CH₃)₂, —NHC(O)CH₂CH(CH₃)₂, —NHC(O)CH(CH₃)CH₂CH₃, —NHC(O)—C(CH₃)₃ and —NHC(O)CH₂C(CH₃)₃.

“Alkylene”, “alkenylene”, and “alkynylene” refers to the subsets of alkyl, alkenyl and alkynyl groups, as defined herein, including the same residues as alkyl, alkenyl, and alkynyl, but having two points of attachment within a chemical structure. Examples of C₁-C₆alkylene include ethylene (—CH₂CH₂—), propylene (—CH₂CH₂CH₂—), and dimethylpropylene (—CH₂C(CH₃)₂CH₂—). Likewise, examples of C₂-C₆alkenylene include ethenylene (—CH═CH— and propenylene (—CH═CH—CH₂—). Examples of C₂-C₆alkynylene include ethynylene (—C≡C—) and propynylene (—C≡C—CH₂—).

“Alkylthio” refers to an alkyl group as defined above, attached to the parent structure through a sulfur atom. Examples of an C₁-C₆alkylthio group include methylthio, ethylthio, n-propylthio, i-propylthio, n-butylthio, i-butylthio, s-butylthio, t-butylthio, n-pentylthio and n-hexylthio.

“Alkynyl” refers to a straight or branched chain unsaturated hydrocarbon containing 2-10 carbon atoms and at least one triple bond. Examples of a C₂-C₁₀ alkynyl group include, but are not limited to, acetylene, propyne, 1-butyne, 2-butyne, isobutyne, sec-butyne, 1-pentyne, 2-pentyne, isopentyne, 1-hexyne, 2-hexyne, 3-hexyne, isohexyne, 1-heptyne, 2-heptyne, 3-heptyne, 1-octyne, 2-octyne, 3-octyne, 4-octyne, 1-nonyne, 2-nonyne, 3-nonyne, 4-nonyne, 1-decyne, 2-decyne, 3-decyne, 4-decyne and 5-decyne. An alkynyl group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, and C₃-C₈cycloalkyl.

“Amido(aryl)-” refers to an aryl group, as defined below, wherein one of the aryl group's hydrogen atoms has been replaced with one or more —C(O)NH₂ groups. Representative examples of an amido(C₆-C₁₄aryl) group include 2-C(O)NH₂-phenyl, 3-C(O)NH₂-phenyl, 4-C(O)NH₂-phenyl, 1-C(O)NH₂-naphthyl, and 2-C(O)NH₂-naphthyl.

“Amino(alkyl)-” refers to a C₁-C₆alkyl group, as defined above, wherein one or more of the C₁-C₆alkyl group's hydrogen atoms has been replaced with —NH₂. Representative examples of an amino(C₁-C₆alkyl) group include, but are not limited to —CH₂NH₂, —CH₂CH₂NH₂, —CH₂CH₂CH₂ NH₂, —CH₂CH₂CH₂CH₂NH₂, —CH₂CH(NH₂)CH₃, —CH₂CH(NH₂)CH₂CH₃, —CH(NH₂)CH₂CH₃ and —C(CH₃)₂(CH₂NH₂), —CH₂CH₂CH₂CH₂CH₂NH₂, and —CH₂CH₂CH(NH₂)CH₂CH₃. An amino(C₁-C₆alkyl) group can be unsubstituted or substituted with one or two of the following groups C₁-C₆alkoxy, C₆-C₁₄aryl, C₁-C₉heteroaryl, C₃-C₈cycloalkyl, and C₁-C₆alkyl.

Aryl refers to an aromatic hydrocarbon group containing 6-14 carbon ring atoms. “C₆-C₁₄Aryl” refers to a phenyl, naphthyl, biphenyl, anthryl, tetrahydronaphthyl, fluorenyl, indanyl, biphenylenyl, and acenaphthenyl, groups. Examples of an C₆-C₁₄aryl group include, but are not limited to, phenyl, 1-naphthyl, 2-naphthyl, and 3-biphen-1-yl. An aryl group can be unsubstituted or substituted with one or more of the following groups: C₁-C₆alkyl, halo, haloalkyl-, hydroxyl, hydroxyl(C₁-C₆alkyl)-, —NH₂, aminoalkyl-, dialkylamino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

“(Aryl)alkyl” refers to alkyl group, as defined above, wherein one or more of the alkyl group's hydrogen atoms has been replaced with an C₆-C₁₄aryl group as defined above. (C₆-C₁₄Aryl)alkyl moieties include benzyl, 1-phenylethyl, 2-phenylethyl, 3-phenylpropyl, 2-phenylpropyl, 1-naphthylmethyl, 2-naphthylmethyl and the like. An (aryl)alkyl group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), carboxyamidoalkyl-, or —NO₂.

“(Aryl)amino” refers to a radical of formula aryl-NH—, wherein “aryl” is as defined above. Examples of (C₆-C₁₄aryl)amino radicals include, but are not limited to, phenylamino (anilido), 1-naphthlamino, 2-naphthlamino and the like. An arylamino group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl.

“(Aryl)oxy” refers to the group Ar—O— where Ar is an aryl group, as defined above. Exemplary (C₆-C₁₄aryl)oxy groups include but are not limited to phenyloxy, α-naphthyloxy, and β-naphthyloxy. An (aryl)oxy group can be unsubstituted or substituted with one or more of the following groups: C₁-C₆alkyl, halo, haloalkyl-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

A “Cycloalkyl” refers to a monocyclic, non-aromatic, saturated hydrocarbon ring containing 3-8 carbon atoms. Representative examples of a C₃-C₈cycloalkyl include, but are not limited to, cyclopropyl, cyclobutyl, cyclopentyl, cyclohexyl, cycloheptyl and cyclooctyl. A cycloalkyl group can be unsubstituted or independently substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl, haloalkyl-, aminoalkyl-, —OC(O)(C₁-C₆alkyl), carboxyamidoalkyl-, or —NO₂. Additionally, each of any two hydrogen atoms on the same carbon atom of the cycloalkyl ring can be replaced by an oxygen atom to form an oxo (═O) substituent or the two hydrogen atoms can be replaced by an alkylenedioxy group so that the alkylenedioxy group, when taken together with the carbon atom to which it is attached, form a 5- to 7-membered heterocycle containing two oxygen atoms.

A “Bicyclic cycloalkyl” refers to a bicyclic, non-aromatic, saturated hydrocarbon ring system containing 6-10 carbon atoms. Representative examples of a C₆-C₁₀bicyclic cycloalkyl include, but are not limited to, cis-1-decalinyl, trans 2-decalinyl, cis-4-perhydroindanyl, and trans-7-perhydroindanyl. A bicyclic cycloalkyl can be unsubstituted or independently substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl, haloalkyl-, aminoalkyl-, —OC(O)(C₁-C₆alkyl), carboxyamidoalkyl-, or —NO₂. Additionally, each of any two hydrogen atoms on the same carbon atom of the bicyclic cycloalkyl rings can be replaced by an oxygen atom to form an oxo (═O) substituent or the two hydrogen atoms can be replaced by an alkylenedioxy group so that the alkylenedioxy group, when taken together with the carbon atom to which it is attached, form a 5- to 7-membered heterocycle containing two oxygen atoms.

A “Carboxyamidoalkyl-” refers to a primary carboxyamide (CONH₂), a secondary carboxyamide (CONHR′) or a tertiary carboxyamide (CONR′R″), where R′ and R″ are the same or different substituent groups selected from C₁-C₆alkyl, C₂-C₆alkenyl, C₂-C₆alkynyl, C₆-C₁₄aryl, C₁-C₉heteroaryl, or C₃-C₈cycloalkyl, attached to the parent compound by an alkylene group as defined above. Exemplary C₁-C₆carboxyamidoalkyl- groups include but are not limited to NH₂C(O)—CH₂—, CH₃NHC(O)—CH₂CH₂—, (CH₃)₂NC(O)—CH₂CH₂CH₂—, CH₂═CHCH₂NHC(O)—CH₂CH₂CH₂CH₂—, HCCCH₂NHC(O)—CH₂CH₂CH₂CH₂CH₂—, C₆H₅NHC(O)—CH₂CH₂CH₂CH₂CH₂CH₂—, 3-pyridylNHC(O)—CH₂CH(CH₃)CH₂CH₂—, and cyclopropyl-CH₂NHC(O)—CH₂CH₂C(CH₃)₂CH₂—.

“Cycloalkenyl” refers to non-aromatic carbocyclic rings containing 3-10 carbon atoms with one or more carbon-to-carbon double bonds within the ring system. The “cycloalkenyl” may be a single ring or may be multi-ring. Multi-ring structures may be bridged or fused ring structures. A cycloalkenyl group can be unsubstituted or independently substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl, haloalkyl-, aminoalkyl-, —OC(O)(C₁-C₆alkyl), carboxyamidoalkyl-, or —NO₂ Additionally, each of any two hydrogen atoms on the same carbon atom of the cycloalkenyl rings may be replaced by an oxygen atom to form an oxo (═O) substituent or the two hydrogen atoms may be replaced by an alkylenedioxy group so that the alkylenedioxy group, when taken together with the carbon atom to which it is attached, form a 5- to 7-membered heterocycle containing two oxygen atoms. Examples of C₃-C₁₀cycloalkenyls include, but are not limited to, cyclopropenyl, cyclobutenyl, cyclopentenyl, cyclohexenyl, 4,4a-octalin-3-yl, and cyclooctenyl.

“Di(alkyl)amino-” refers to a nitrogen atom which has attached to it two alkyl groups, as defined above. Each alkyl group can be independently selected from the C₁-C₆alkyl groups. Representative examples of an di(C₁-C₆alkyl)amino- group include, but are not limited to, —N(CH₃)₂, —N(CH₂CH₃)(CH₃), —N(CH₂CH₃)₂, —N(CH₂CH₂CH₃)₂, —N(CH₂CH₂CH₂CH₃)₂, —N(CH(CH₃)₂)₂, —N(CH(CH₃)₂)(CH₃), —N(CH₂CH(CH₃)₂)₂, —NH(CH(CH₃)CH₂CH₃)₂, —N(C(CH₃)₃)₂, —N(C(CH₃)₃)(CH₃), and —N(CH₃)(CH₂CH₃). The two alkyl groups on the nitrogen atom, when taken together with the nitrogen to which they are attached, can form a 3- to 7-membered nitrogen containing heterocycle wherein up to two of the carbon atoms of the heterocycle can be replaced with —N(R)—, —O—, or —S(O)ₙ—. R is hydrogen, C₁-C₆alkyl, C₃-C₈cycloalkyl, C₆-C₁₄aryl, C₁-C₉heteroaryl, amino(C₁-C₆alkyl), or arylamino. Variable n is 0, 1, or 2.

“Halo” and “Halogen” is —F, —Cl, —Br or —I.

“Halo(alkyl)” refers to a alkyl group, as defined above, wherein one or more of the alkyl group's hydrogen atoms has been replaced with —F, —Cl, —Br, or —I. Each substitution can be independently selected from —F, —Cl, —Br, or —I. Representative examples of an halo(C₁-C₆alkyl) group include, but are not limited to —CH₂F, —CCl₃, —CF₃, CH₂CF₃, —CH₂Cl, —CH₂CH₂Br, —CH₂CH₂I, —CH₂CH₂CH₂F, —CH₂CH₂CH₂Cl, —CH₂CH₂CH₂CH₂Br, —CH₂CH₂CH₂CH₂I, —CH₂CH₂CH₂CH₂CH₂Br, —CH₂CH₂CH₂CH₂CH₂I, —CH₂CH(Br)CH₃, —CH₂ CH(Cl)CH₂CH₃, —CH(F)CH₂CH₃ and —C(CH₃)₂(CH₂Cl).

“Heteroaryl” refers to 5-10-membered mono and bicyclic aromatic groups containing at least one heteroatom selected from oxygen, sulfur and nitrogen. Examples of monocyclic heteroaryl radicals include, but are not limited to, oxazinyl, thiazinyl, diazinyl, triazinyl, tetrazinyl, imidazolyl, tetrazolyl, isoxazolyl, furanyl, furazanyl, oxazolyl, thiazolyl, thiophenyl, pyrazolyl, triazolyl, pyrimidinyl, N-pyridyl, 2-pyridyl, 3-pyridyl and 4-pyridyl. Examples of bicyclic heteroaryl radicals include but are not limited to, benzimidazolyl, indolyl, isoquinolinyl, indazolyl, quinolinyl, quinazolinyl, purinyl, benzisoxazolyl, benzoxazolyl, benzthiazolyl, benzodiazolyl, benzotriazolyl, isoindolyl and indazolyl. A heteroaryl group can be unsubstituted or substituted with one or more of the following groups: C₁-C₆alkyl, halo, haloalkyl-, hydroxyl, hydroxyl(C₁-C₆alkyl)-, —NH₂, aminoalkyl-, dialkylamino-, —COOH, —C(O)O—(C₁-C₆alkyl), —OC(O)(C₁-C₆alkyl), N-alkylamido-, —C(O)NH₂, (C₁-C₆alkyl)amido-, or —NO₂.

“(C₁-C₉Heteroaryl)oxy” refers to the group Het-O— where Het is a heteroaryl group, as defined above. Exemplary alkoxy groups include but are not limited to pyridin-2-yloxy, pyridin-3-yloxy, pyrimidin-4-yloxy, and oxazol-5-yloxy. A (C₁-C₉heteroaryl)oxy group can be unsubstituted or substituted with one or more of the following groups: C₁-C₆alkyl, halo, haloalkyl-, hydroxyl, hydroxyl(C₁-C₆alkyl)-, —NH₂, aminoalkyl-, dialkylamino-, —COOH, —C(O)O—(C₁-C₆alkyl), OC(O)(C₁-C₆alkyl), N-alkylamido-, —C(O)NH₂, (C₁-C₆alkyl)amido-, or —NO₂.

The term “heteroatom” refers to a sulfur, nitrogen, or oxygen atoms.

“Heterocycle” refers to 3-10-membered mono and bicyclic groups containing at least one heteroatom selected from oxygen, sulfur and nitrogen. A heterocycle may be saturated, aromatic, or partially saturated. Exemplary C₁-C₉heterocycle groups include but are not limited to aziridine, oxirane, oxirene, thiirane, pyrroline, pyrrolidine, pyrrole, dihydrofuran, tetrahydrofuran, furan, dihydrothiophene, tetrahydrothiophene, thiophene, pyrazole, imidazole, triazole, tetrazole, oxazole, isoxazole, thiazole, isothiazole, dithiolane, piperidine, pyridine, tetrahydropyran, pyran, thiane, thiine, piperazine, oxazine, thiazine, dithiane, dioxane, pyrazine, pyrimidine, pyridazine, quinoline, isoquinoline, purine, and quinazoline.

“Heterocyclyl(alkyl)” refers to a alkyl group, as defined above, wherein one or more of the alkyl group's hydrogen atoms has been replaced with a heterocycle group as defined above. Heterocyclyl(C₁-C₆alkyl) moieties include 2-pyridylmethyl, 1-piperazinylethyl, 2-thiophenylethyl, 4-morpholinylpropyl, 3-pyridylpropyl, 2-quinolinylmethyl, 2-indolylmethyl and the like. An heterocyclyl(alkyl) group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), 4- to 7-membered monocyclic heterocycle, C₆-C₁₄aryl, C₁-C₉heteroaryl, or C₃-C₈cycloalkyl.

“Hydroxylalkyl-” refers to a alkyl group, as defined above, wherein one or more of the alkyl group's hydrogen atoms has been replaced with hydroxyl groups. Examples of C₁-C₆hydroxylalkyl- moieties include, for example, —CH₂OH, —CH₂CH₂OH, —CH₂CH₂CH₂OH, —CH₂CH(OH)CH₂OH, —CH₂CH(OH)CH₃, —CH(CH₃)CH₂OH and higher homologs.

The term “monocyclic heterocycle” refers to a monocyclic 3- to 7-membered aromatic, cycloalkyl, or cycloalkenyl in which 1-4 of the ring carbon atoms have been independently replaced with an N, O or S atom. The monocyclic heterocyclic ring can be attached via a nitrogen, sulfur, or carbon atom. Representative examples of a 3- to 7-membered monocyclic heterocycle group include, but are not limited to, piperidinyl, 1,2,5,6-tetrahydropyridiyl, piperazinyl, morpholinyl, pyrrolyl, oxazinyl, thiazinyl, diazinyl, triazinyl, tetrazinyl, imidazolyl, tetrazolyl, pyrrolidinyl, isoxazolyl, furanyl, furazanyl, pyridinyl, oxazolyl, thiazolyl, thiophenyl, pyrazolyl, triazolyl, and pyrimidinyl. A monocyclic heterocycle group can be unsubstituted or substituted with one or more of the following groups: C₁-C₈acyl, C₁-C₆alkyl, heterocyclylalkyl, (C₆-C₁₄aryl)alkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂.

“Bicyclic heterocycle” refers to a bicyclic aromatic, bicyclic cycloalkyl, or bicyclic cycloalkenyl in which 1-4 of the ring carbon atoms have been independently replaced with an N, O or S atom. The bicyclic heterocyclic ring can be attached via a nitrogen, sulfur, or carbon atom. Representative examples of a 6- to 10-membered bicyclic heterocycle group include, but are not limited to, benzimidazolyl, indolyl, indolinyl, isoquinolinyl, indazolyl, quinolinyl, tetrahydroquinolinyl, quinazolinyl, purinyl, benzisoxazolyl, benzoxazolyl, benzthiazolyl, benzodiazolyl, benzotriazolyl, isoindolyl and indazolyl. A bicyclic heterocycle group can be unsubstituted or substituted with one or more of the following groups: C₁-C₈acyl, C₁-C₆alkyl, C₁-C₆heterocyclylalkyl, (C₆-C₁₄aryl)alkyl, halo, C₁-C₆haloalkyl-, hydroxyl, C₁-C₆hydroxylalkyl-, —NH₂, aminoalkyl-, -dialkylamino-, —COOH, —C(O)O—(C₁-C₆alkyl), —OC(O)(C₁-C₆alkyl), (C₆-C₁₄aryl)alkyl-O—C(O)—, N-alkylamido-, —C(O)NH₂, (C₁-C₆alkyl)amido-, or —NO₂.

The term “perfluoroalkyl” refers to both straight- and branched-chain alkyl groups having at least one carbon atom and two or more fluorine atoms. Examples of a C₁-C₆ perfluoroalkyl- include CF₃, CH₂CF₃, CF₂CF₃ and CH(CF₃)₂.

The term “optionally substituted” as used herein means that at least one hydrogen atom of the substituted group has been substituted with halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl.

A “subject” is a mammal, e.g., a human, mouse, rat, guinea pig, dog, cat, horse, cow, pig, or non-human primate, such as a monkey, chimpanzee, baboon or gorilla.

The dashed line - - - - - represents an optional second carbon-to-carbon bond. For example, in a formula [symbol] would be a either carbon-to-carbon double bond or a carbon to carbon single bond with two hydrogen atoms present to complete the normal quadrivalency of carbon.

The compounds of the present invention exhibit an mTOR inhibitory activity and therefore, can be utilized in order to inhibit abnormal cell growth in which mTOR plays a role. Thus, the compounds of the present invention are effective in the treatment of disorders with which abnormal cell growth actions of mTOR are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the compounds of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovary cancer, prostate cancer, lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

When administered to an animal, the compounds of the present invention or pharmaceutically acceptable salts thereof can be administered neat or as a component of a composition that comprises a pharmaceutically acceptable carrier or vehicle. A composition of the invention can be prepared using a method comprising admixing the compound of the present invention or pharmaceutically acceptable salt thereof and a physiologically acceptable carrier, excipient, or diluent. Admixing can be accomplished using methods well known in the art.

The present compositions, comprising compounds of the present invention or pharmaceutically acceptable salts thereof can be administered orally, or by any other convenient route, for example, by infusion or bolus injection, by absorption through epithelial or mucocutaneous linings (e.g., oral, rectal, vaginal, and intestinal mucosa, etc.) and can be administered together with another therapeutic agent. Administration can be systemic or local. Various known delivery systems, including encapsulation in liposomes, microparticles, microcapsules, and capsules, can be used.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result of release of the compound of the present invention or pharmaceutically acceptable salt thereof into the bloodstream. The mode of administration is left to the discretion of the practitioner.

In one aspect, the compound of the present invention or pharmaceutically acceptable salt thereof is administered orally.

In another aspect, the compound of the present invention or pharmaceutically acceptable salt thereof is administered intravenously.

In another aspect, it can be desirable to administer the compound of the present invention or pharmaceutically acceptable salt thereof locally. This can be achieved, for example, by local infusion during surgery, topical application, e.g., in conjunction with a wound dressing after surgery, by injection, by means of a catheter, by means of a suppository or edema, or by means of an implant, said implant being of a porous, non-porous, or gelatinous material, including membranes, such as sialastic membranes, or fibers.

In certain aspects, it can be desirable to introduce the compound of the present invention or pharmaceutically acceptable salt thereof into the central nervous system, circulatory system or gastrointestinal tract by any suitable route, including intraventricular, intrathecal injection, paraspinal injection, epidural injection, enema, and by injection adjacent to the peripheral nerve. An intraventricular catheter, for example, can facilitate intraventricular injection attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain aspects, the compound of the present invention or pharmaceutically acceptable salt thereof can be formulated as a suppository, with traditional binders and excipients such as triglycerides.

In another aspect, compound of the present invention or pharmaceutically acceptable salt thereof can be delivered in a vesicle, in particular a liposome by methods known in the art.

In yet another aspect, the compound of the present invention or pharmaceutically acceptable salt thereof can be delivered in a controlled-release system or sustained-release system by methods known in the art. In one aspect, a pump can be used. In another aspect, polymeric materials can be used.

In yet another aspect, a controlled- or sustained-release system can be placed in proximity of a target of the compound of the present invention or a pharmaceutically acceptable salt thereof, e.g., the reproductive organs, thus requiring only a fraction of the systemic dose.

The present compositions can optionally comprise a suitable amount of a pharmaceutically acceptable excipient.

Such pharmaceutically acceptable excipients can be liquids, such as water and oils, including those of petroleum, animal, vegetable, or synthetic origin, such as peanut oil, soybean oil, mineral oil, sesame oil and the like. The excipients can be saline, gum acacia, gelatin, starch paste, talc, keratin, colloidal silica, urea and the like. In addition, auxiliary, stabilizing, thickening, lubricating, and coloring agents can be used. In one aspect, the excipients are sterile when administered to an animal. The excipient should be stable under the conditions of manufacture and storage and should be preserved against the contaminating action of microorganisms. Water is a particularly useful excipient in the practice of this invention where administration is performed intravenously. Saline solutions and aqueous dextrose and glycerol solutions can also be employed as liquid excipients, particularly for injectable solutions. Suitable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents known in the art.

Liquid carriers may be used in preparing solutions, suspensions, emulsions, syrups, and elixirs. The compound of the present invention or pharmaceutically acceptable salt thereof can be dissolved or suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particular containing additives as above, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one aspect, the composition is in the form of a capsule.

In one aspect, the compound of the present invention or pharmaceutically acceptable salt thereof is formulated in accordance with known procedures as a composition adapted for oral administration to humans. Compositions for oral delivery can be in the form of tablets, lozenges, buccal forms, troches, aqueous or oily suspensions or solutions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. In powders, the carrier can be a finely divided solid, which is an admixture with the finely divided compound of the present invention or pharmaceutically acceptable salt thereof. In tablets, the compound of the present invention or pharmaceutically acceptable salt thereof is mixed with a carrier having the necessary compression properties in suitable proportions and compacted in the shape and size desired. The powders and tablets can contain up to about 99% of the compound of the present invention or pharmaceutically acceptable salt thereof.

Capsules may contain mixtures of the compounds of the present invention or pharmaceutically acceptable salts thereof with inert fillers and/or diluents such as pharmaceutically acceptable starches (e.g., corn, potato, or tapioca starch), sugars, artificial sweetening agents, powdered celluloses (such as crystalline and microcrystalline celluloses), flours, gelatins, gums, etc.

Tablet formulations can be made by conventional compression, wet granulation, or dry granulation methods and utilize pharmaceutically acceptable diluents, binding agents, lubricants, disintegrants, surface modifying agents (including surfactants), suspending or stabilizing agents (including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrroldine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine.

Moreover, when in a tablet or pill form, the compositions can be coated to delay disintegration and absorption in the gastrointestinal tract, thereby providing a sustained action over an extended period of time. Selectively permeable membranes surrounding an osmotically active driving compound or a pharmaceutically acceptable salt of the compound are also suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one aspect, the excipients are of pharmaceutical grade.

In another aspect, the compound of the present invention or pharmaceutically acceptable salt thereof can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where the compound of the present invention or pharmaceutically acceptable salt thereof is to be administered by infusion, it can be dispensed, for example, with an infusion bottle containing sterile pharmaceutical grade water or saline. Where the compound of the present invention or pharmaceutically acceptable salt thereof is administered by injection, an ampoule of sterile water for injection or saline can be provided so that the ingredients can be mixed prior to administration.

In another aspect, the compound of the present invention or pharmaceutically acceptable salt thereof can be administered transdermally through the use of a transdermal patch. Transdermal administrations include administrations across the surface of the body and the inner linings of the bodily passages including epithelial and mucosal tissues. Such administrations can be carried out using the present compounds of the present invention or pharmaceutically acceptable salts thereof, in lotions, creams, foams, patches, suspensions, solutions, and suppositories (e.g., rectal or vaginal).

Transdermal administration can be accomplished through the use of a transdermal patch containing the compound of the present invention or pharmaceutically acceptable salt thereof and a carrier that is inert to the compound of the present invention or pharmaceutically acceptable salt thereof, is non-toxic to the skin, and allows delivery of the agent for systemic absorption into the blood stream via the skin. The carrier may take any number of forms such as creams or ointments, pastes, gels, or occlusive devices. The creams or ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the compound of the present invention or pharmaceutically acceptable salt thereof into the blood stream, such as a semi-permeable membrane covering a reservoir containing the compound of the present invention or pharmaceutically acceptable salt thereof with or without a carrier, or a matrix containing the active ingredient.

The compounds of the present invention or pharmaceutically acceptable salts thereof may be administered rectally or vaginally in the form of a conventional suppository. Suppository formulations may be made from traditional materials, including cocoa butter, with or without the addition of waxes to alter the suppository's melting point, and glycerin. Water-soluble suppository bases, such as polyethylene glycols of various molecular weights, may also be used.

The compound of the present invention or pharmaceutically acceptable salt thereof can be administered by controlled-release or sustained-release means or by delivery devices that are known to those of ordinary skill in the art. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release. Advantages of controlled- or sustained-release compositions include extended activity of the drug, reduced dosage frequency, and increased compliance by the animal being treated. In addition, controlled- or sustained-release compositions can favorably affect the time of onset of action or other characteristics, such as blood levels of the compound of the present invention or a pharmaceutically acceptable salt thereof, and can thus reduce the occurrence of adverse side effects.

Controlled- or sustained-release compositions can initially release an amount of the compound of the present invention or pharmaceutically acceptable salt thereof that promptly produces the desired therapeutic or prophylactic effect, and gradually and continually release other amounts of the compound of the present invention or pharmaceutically acceptable salt thereof to maintain this level of therapeutic or prophylactic effect over an extended period of time.

In certain aspects, the present invention is directed to prodrugs of the compounds of the present invention or pharmaceutically acceptable salts of compounds of the present invention of the present invention. Various forms of prodrugs are known in the art.

The amount of the compound of the present invention or pharmaceutically acceptable salt thereof that is effective for inhibiting mTOR or PI3K in a subject. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner.

The amount of the compound of the present invention or pharmaceutically acceptable salt thereof that is effective for treating or preventing an mTOR-related disorder will typically range from about 0.001 mg/kg to about 250 mg/kg of body weight per day, in one aspect, from about 1 mg/kg to about 250 mg/kg body weight per day, in another aspect, from about 1 mg/kg to about 50 mg/kg body weight per day, and in another aspect, from about 1 mg/kg to about 20 mg/kg of body weight per day.

In one aspect, the pharmaceutical composition is in unit dosage form, e.g., as a tablet, capsule, powder, solution, suspension, emulsion, granule, or suppository. In such form, the composition is sub-divided in unit dose containing appropriate quantities of the active ingredient; the unit dosage form can be packaged compositions, for example, packeted powders, vials, ampoules, prefilled syringes or sachets containing liquids. The unit dosage form can be, for example, a capsule or tablet itself, or it can be the appropriate number of any such compositions in package form. Such unit dosage form may contain from about 1 mg/kg to about 250 mg/kg, and may be given in a single dose or in two or more divided doses.

The present methods for treating or preventing an mTOR-related disorder, can further comprise administering another therapeutic agent to the animal being administered the compound of the present invention or pharmaceutically acceptable salt thereof. In one aspect, the other therapeutic agent is administered in an effective amount.

Effective amounts of other therapeutic agents to be administered simultaneously or sequentially with the present compound or pharmaceutically acceptable salt thereof are well known to those skilled in the art. However, it is well within the skilled artisan's purview to determine the other therapeutic agent's optimal effective amount range.

Suitable other therapeutic agents useful in the methods and compositions of the present invention include, but are not limited to temozolomide, a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, hydroxyzine, glatiramer acetate, interferon beta-1a, interferon beta-1b, mitoxantrone, natalizumab, L-asparaginase, doxorubicin, epirubicin, 5-fluorouracil, taxanes such as docetaxel and paclitaxel, leucovorin, levamisole, irinotecan, estramustine, etoposide, nitrogen mustards, BCNU, nitrosoureas such as carmustine and lomustine, vinca alkaloids such as vinblastine, vincristine and vinorelbine, platinum complexes such as cisplatin, carboplatin and oxaliplatin, imatinib mesylate, hexamethylmelamine, topotecan, tyrosine kinase inhibitors, tyrphostins herbimycin A, genistein, erbstatin, and lavendustin A.

In one aspect, the compound of the present invention or pharmaceutically acceptable salt thereof is administered concurrently with another therapeutic agent.

In one aspect, a composition comprising an effective amount of the compound of the present invention or pharmaceutically acceptable salt thereof and an effective amount of another therapeutic agent within the same composition can be administered.

In another aspect, a composition comprising an effective amount of the compound of the present invention or a pharmaceutically acceptable salt of the compound of the present invention and a separate composition comprising an effective amount of another therapeutic agent can be concurrently administered. In another aspect, an effective amount of the compound of the present invention or a pharmaceutically acceptable salt of the compounds of the present invention administered prior to or subsequent to administration of an effective amount of another therapeutic agent.

The invention further comprises a method of treating advanced renal cell carcinoma, comprising administering to a mammal in need thereof the compounds or a pharmaceutically acceptable salt of the compounds of the present invention in an amount effective to treat advanced renal cell carcinoma.

Another aspect is a method of treating acute lymphoblastic leukemia, comprising administering to a mammal in need thereof the compounds or a pharmaceutically acceptable salt of the compounds of any of the present invention in an amount effective to treat acute lymphoblastic leukemia.

Another aspect is a method of treating acute lymphoblastic leukemia, comprising administering to a mammal in need thereof the compounds or a pharmaceutically acceptable salt of the compounds of any of the present invention in an amount effective to treat malignant melanoma.

Another aspect is a method of treating acute lymphoblastic leukemia, comprising administering to a mammal in need thereof the compounds or a pharmaceutically acceptable salt of the compounds of any of the present invention in an amount effective to treat soft-tissue or bone sarcoma.

Methods useful for making the compounds of the present invention are set forth in the Examples below and generalized in Schemes 1-14:

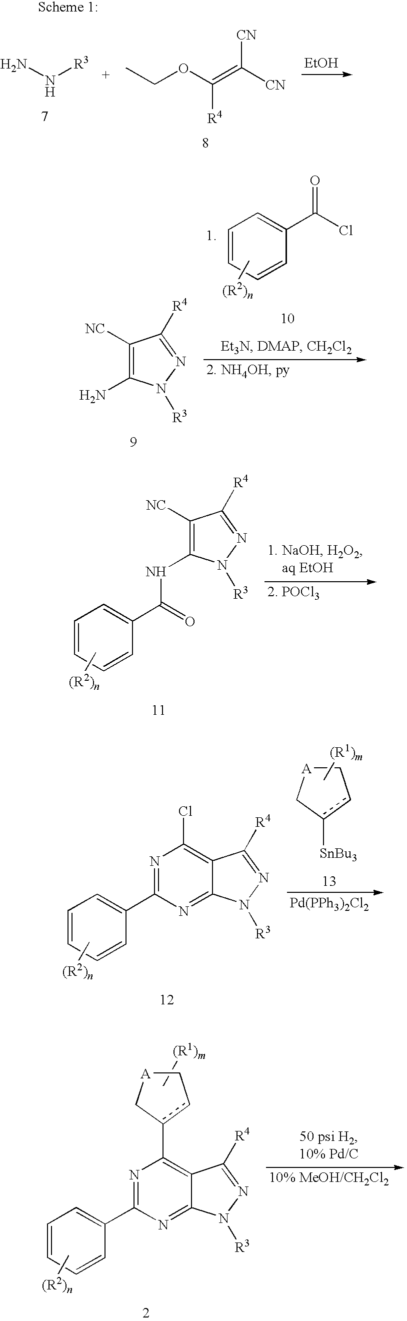

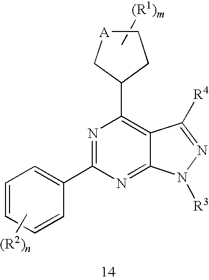

1H-Pyrazolo[3,4-d]pyrimidine compounds were prepared by a six-step sequence as depicted in Scheme 1. The hydrazine 7 was reacted with a (methylidene)malononitrile 8 and the resulting pyrazole 9 was subjected to acylation with different acid halides under basic conditions to give the corresponding amide intermediates 11. The pyrimidine ring was formed by oxidative cyclization follow by conversion of the 6-oxo compounds to a 6-halo intermediate 12. Stille coupling with tributyltin reagent 13 gave the desired 6-substituted 1H-pyrazolo[3,4-d]pyrimidine compound 2. If needed, the optional double bond in the substituent at position 6 could be reduced by catalytic hydrogenation to give the desired 1H-Pyrazolo[3,4-d]pyrimidine compounds. 14.

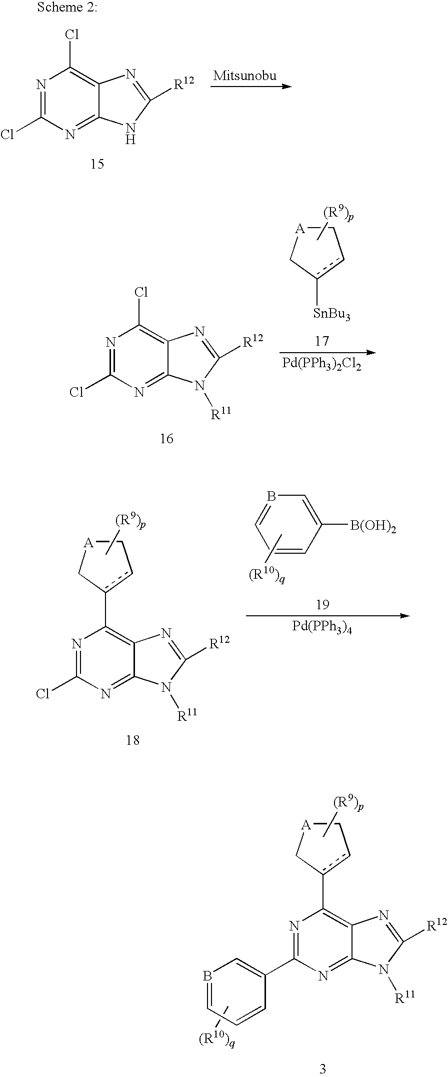

Purine compounds 3 were prepared according to Scheme 2 by a two-step sequence. If needed, the 2,4-dichloro-purine 15 was alkylated at N-9 under typical Mitsunobu conditions. Either substituted purine 16 or non-substituted purine 15 was reacted with the tributyltin reagent 17 under Stille conditions, followed by Suzuki coupling with boronic acid 19 to give the desired purine 3.

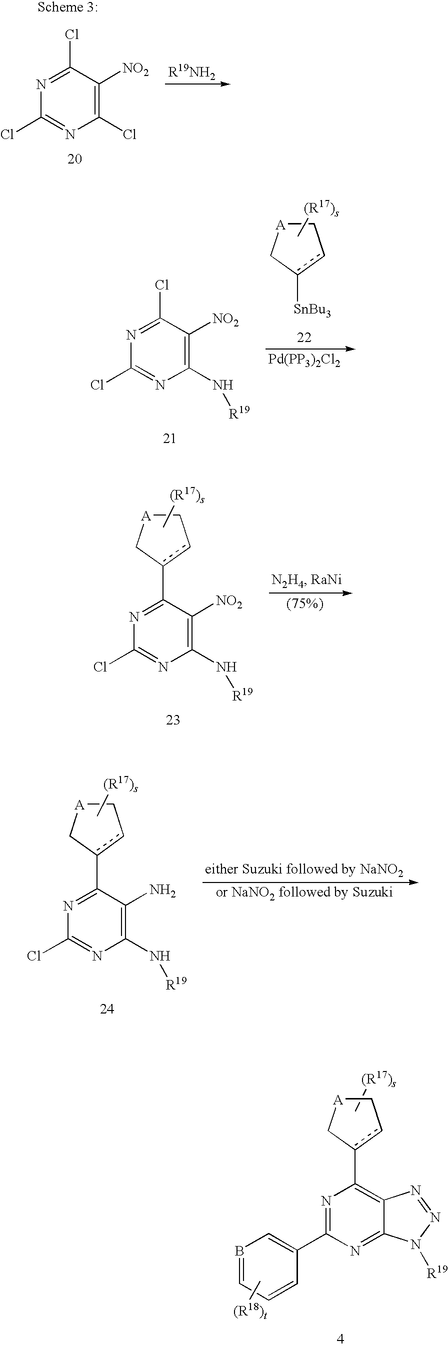

3H-[1,2,3]Triazolo[4,5-d]pyrimidine compounds 4 were prepared by a five-step sequence as depicted in Scheme 3. The commercially available 5-nitro-2,4,6-trichloropyrimidine 20 was reacted with an primary amine R¹⁹NH₂ and the resulting product 21 was subjected to Stille coupling with tributyltin reagent 22, which replaced the halogen atom at position 4 of the pyrimidine ring. Selective reduction of the nitro group at position 5 of the pyrimidine with hydrazine and Raney™ nickel gave the diamine 24. Performing either a two step sequence of Suzuki coupling followed by diazotization and cyclization or a two step sequence of diazotization and cyclization followed by Suzuki coupling gave the desired 3H-[1,2,3]triazolo[4,5-d]pyrimidine 4.

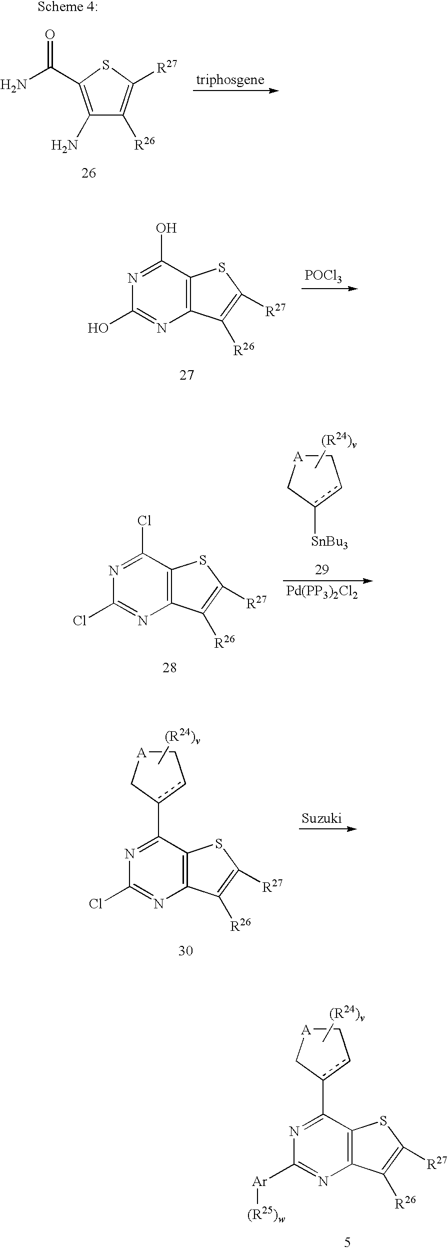

Thieno[3,2-d]pyrimidine compounds 5 were prepared by a four-step sequence as depicted in Scheme 4. The thiophene 26 was reacted with triphosgene (CCl₃OC(O)OCCl₃) or an equivalent to form the fused pyrimidine ring. Conversion of the 2,4-dioxo compounds to a 2,4-dihalo intermediate 28 was done by conventional means. Stille coupling with tributyltin reagent 29 gave the intermediate 4-substituted thieno[3,2-d]pyrimidine compound 30. Suzuki reaction with the appropriate boronic acids or esters under microwave conditions or under thermal conditions to gave the corresponding 2-aryl thieno[3,2-d]pyrimidine 5.

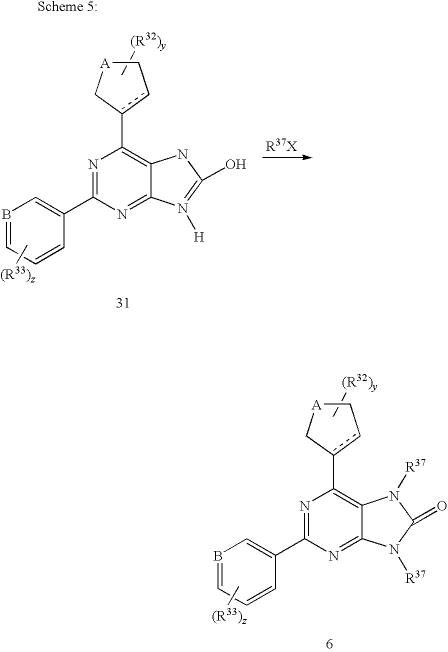

The 7,9-disubstituted 7H-purin-8(9H)-one compounds 6, were made by alkylation of the non-substituted intermediate 31 with an excess of the alkylating agent R³⁷ under basic conditions.

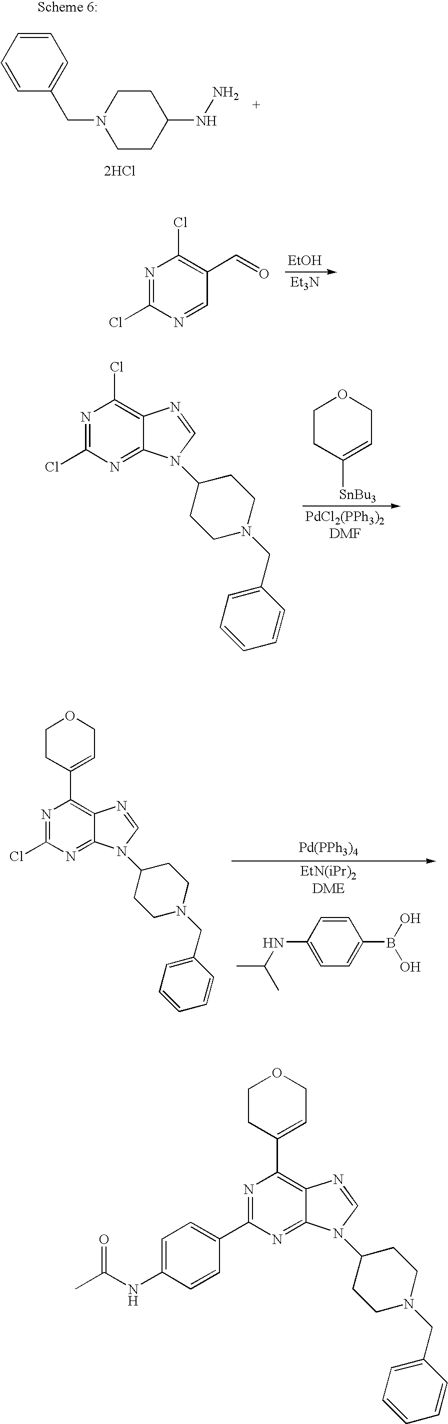

The 1-(piperidin-4-yl)-1H-pyrazolo[3,4-d]pyrimidine compounds were made from 1-benzyl-4-hydrazinylpiperidine and 2,4-dichloropyrimidine-5-carbaldehyde. Stille coupling followed by Suzuki reaction with the appropriate boronic acids or esters gave the desired fully substituted 1H-pyrazolo[3,4-d]pyrimidine compounds as shown in Scheme 6.

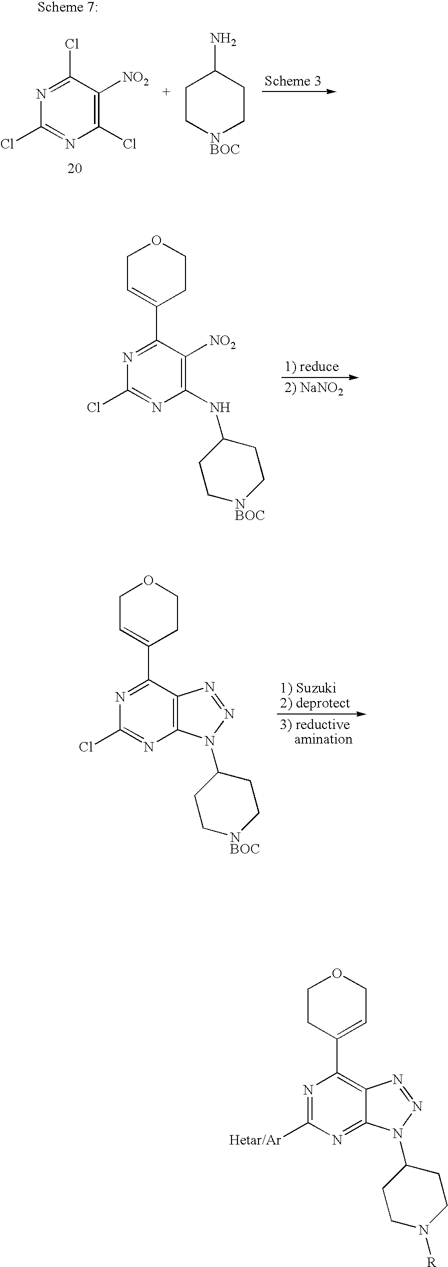

The 3-(piperidin-4-yl)-5-aryl-3H-[1,2,3]triazolo[4,5-d]pyrimidine compounds were prepared from tert-butyl 4-aminopiperidine-1-carboxylate and commercially available 5-nitro-2,4,6-trichloropyrimidine 20 following the procedure outlined in Scheme 3. Selective reduction of the nitro group at position 5 of the pyrimidine with hydrazine and Raney™ nickel, diazotization and cyclization, followed by Suzuki coupling gave a 3H-[1,2,3]triazolo[4,5-d]pyrimidine. Removal of the BOC protecting group and reductive amination of the liberated piperidine allowed ready introduction of substituent R, as outlined in Scheme 7.

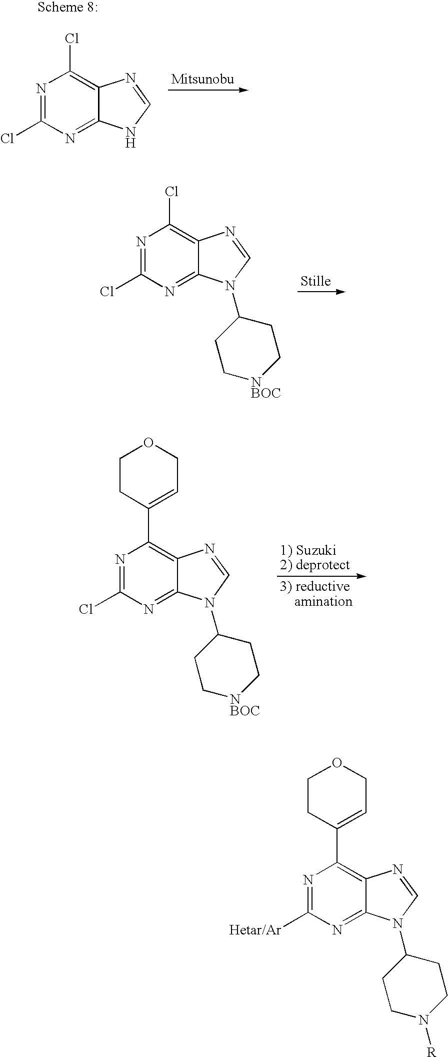

The 2-aryl-9-(piperidin-4-yl)-9H-purine compounds, not substituted at position 8 of the purine ring, were made from the commercially available 2,6-dichloro-9H-purine following the procedure of Scheme 2. 4-Hydroxy-1-piperidine was used under typical Mitsunobu conditions. Reaction with the appropriate tributyltin reagent under Stille conditions, followed by Suzuki coupling with the appropriate boronic acid to gave a BOC-protected purine intermediate. Removal of the BOC protecting group and reductive amination of the liberated piperidine allowed ready introduction of substituent R, as outlined in Scheme 8.

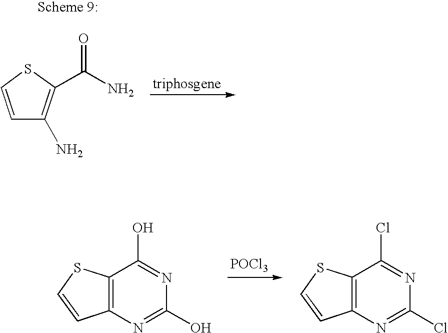

The key intermediate 2,4-dichlorothieno[3,2-d]pyrimidine was made as shown in Scheme 9, following the first two steps of Scheme 4. Reaction with triphosgene (CCl₃OC(O)OCCl₃) or an equivalent formed the fused pyrimidine ring. Conversion of the 2,4-dioxo compounds to a 2,4-dihalo intermediate was done by conventional means.

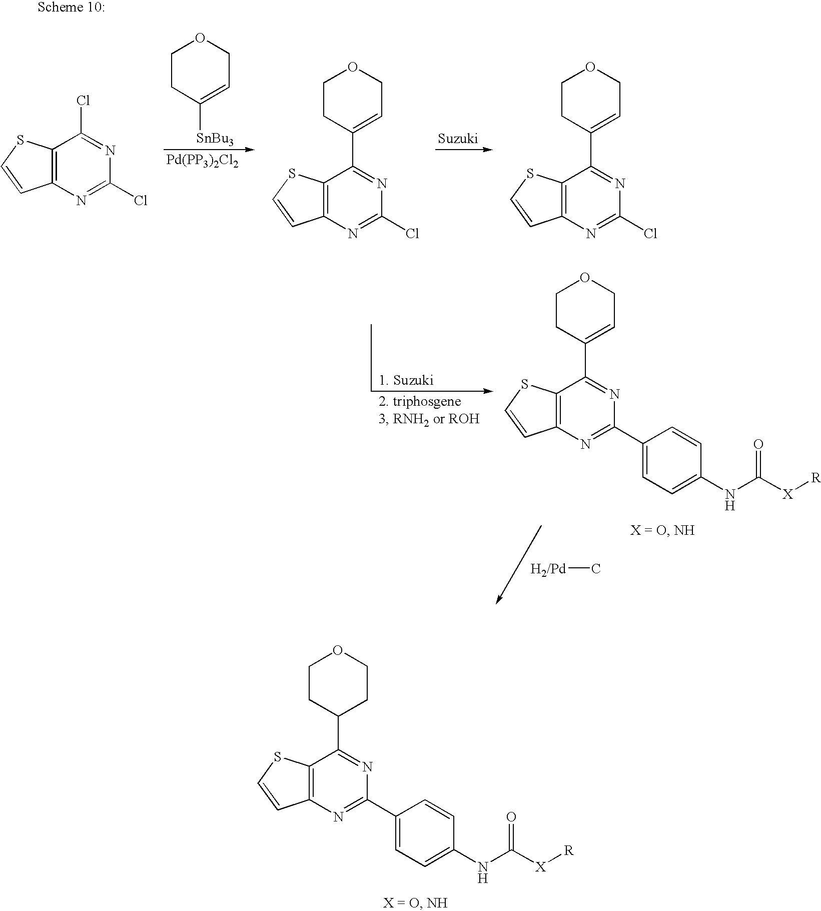

The thieno[3,2-d]pyrimidin-2-yl)phenyl-4-urea and carbamate compounds were made from 2,4-dichlorothieno[3,2-d]pyrimidine as shown in Scheme 10 above, following the last steps of Scheme 4. Stille coupling with the appropriate tributyltin reagent gave the intermediate 2-chloro-thieno[3,2-d]pyrimidine compound. Suzuki reaction with the appropriate boronic acids or esters under microwave conditions or under thermal conditions gave the corresponding 2-aryl thieno[3,2-d]pyrimidine. When 4-aminophenylboronic acid, pinacol ester was used for the Suzuki coupling, then the urea was made by activation with triphosgene (CCl₃OC(O)OCCl₃) or an equivalent followed by reaction with amine RNH₂ or alcohol ROH. Catalytic reduction of the 3,6-dihydro-2H-pyran-4-yl compound gave the tetrahydropyran shown in Scheme 10, if needed.

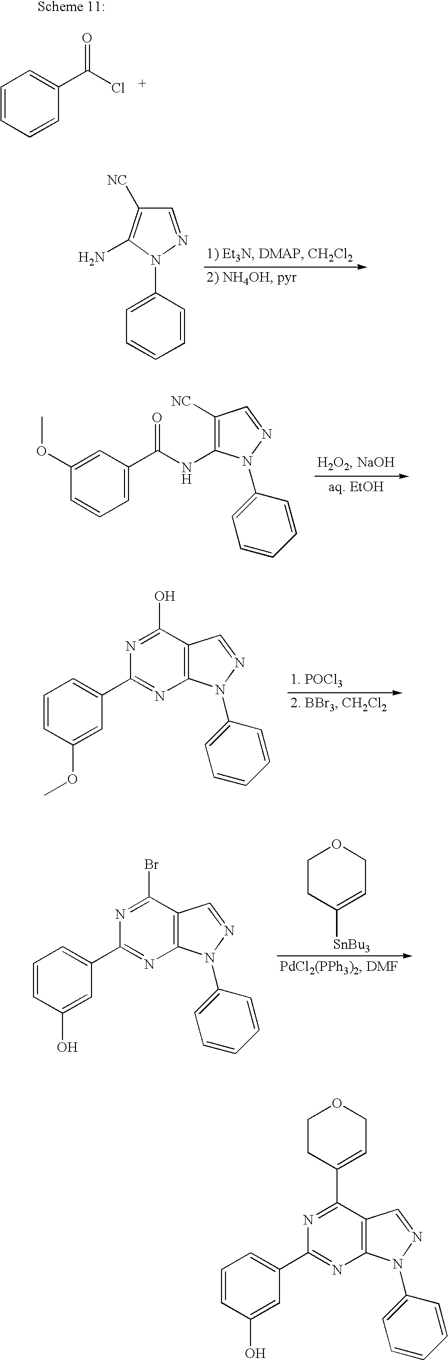

3-(4-(3,6-Dihydro-2H-pyran-4-yl)-1-phenyl-1H-pyrazolo[3,4-d]pyrimidin-6-yl)phenol was made as outlined in Scheme 11. 5-Amino-1-phenyl-1H-pyrazole-4-carbonitrile was acylated with benzoyl chloride following the general procedure of Scheme 1. The pyrimidine ring was formed by oxidative cyclization follow by conversion of the 6-oxo compounds to a 6-halo intermediate. Stille coupling gave the desired 6-substituted 1H-pyrazolo[3,4-d]pyrimidine compound.

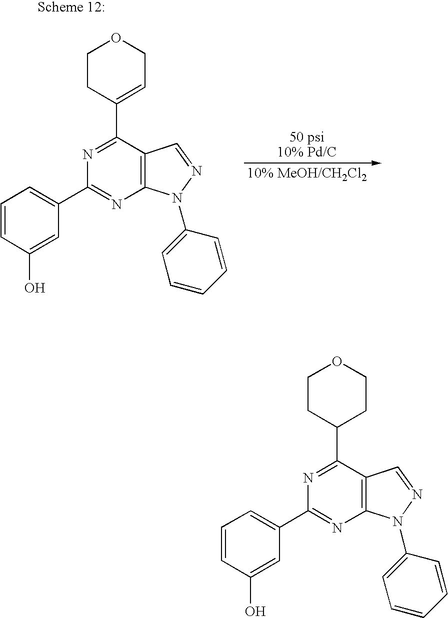

Catalytic reduction of the 3,6-dihydro-2H-pyran-4-yl compound gave the tetrahydropyran shown in Scheme 12, if needed.

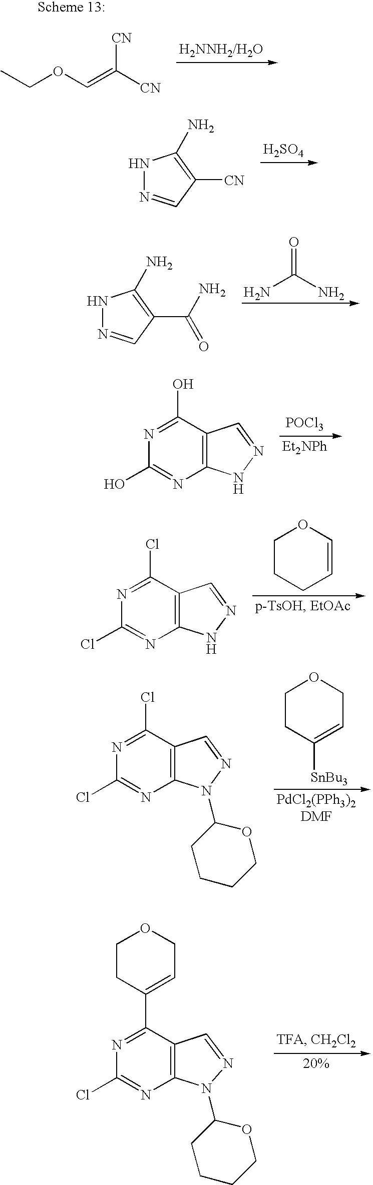

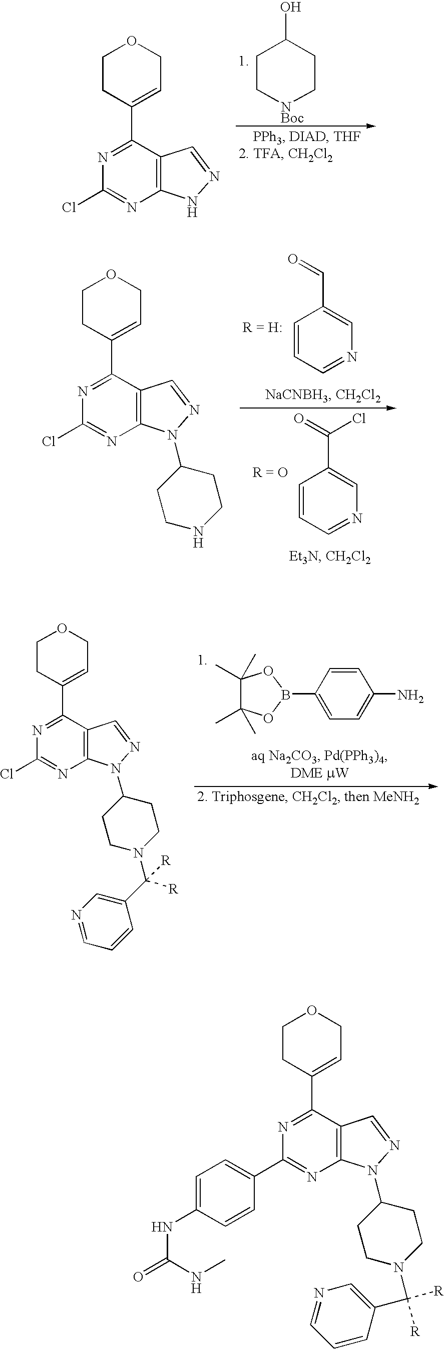

1-(4-(4-(3,6-Dihydro-2H-pyran-4-yl)-1-(1-nicotinoylpiperidin-4-yl)-1H-pyrazolo[3,4-d]pyrimidin-6-yl)phenyl)-3-methylurea and 1-(4-(4-(3,6-dihydro-2H-pyran-4-yl)-1-(1-(pyridin-3-ylmethyl)piperidin-4-yl)-1H-pyrazolo[3,4-d]pyrimidin-6-yl)phenyl)-3-methylurea were made following a variation of Scheme 1. The intermediate 6-chloro-4-(3,6-dihydro-2H-pyran-4-yl)-1-(piperidin-4-yl)-1H-pyrazolo[3,4-d]pyrimidine was either reductively aminated with nicotinaldehyde or acylated with nicotinoyl chloride as appropriate. 4-Aminophenylboronic acid, pinacol ester was used for the Suzuki coupling and the urea was made by activation with triphosgene (CCl₃OC(O)OCCl₃) or an equivalent followed by reaction with methylamine, as shown in Scheme 13.

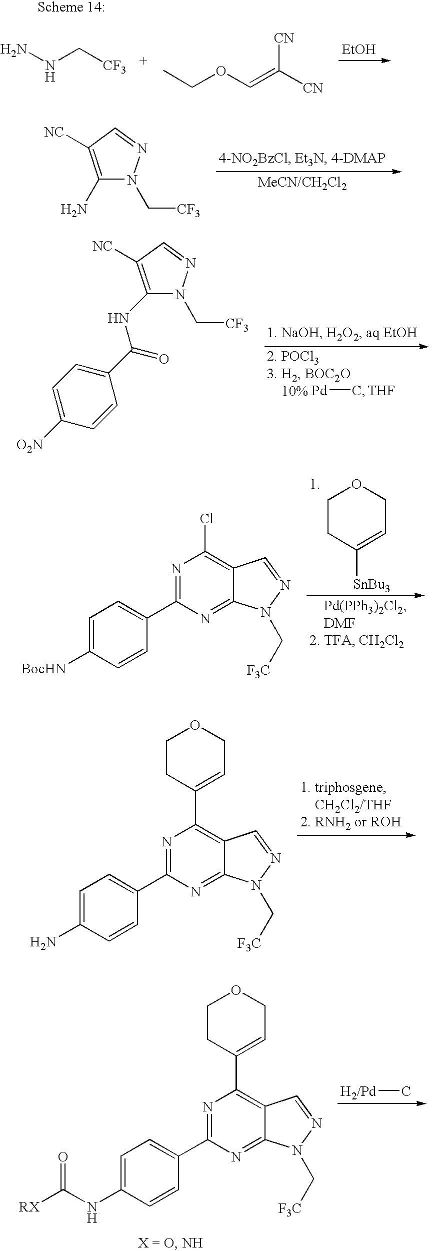

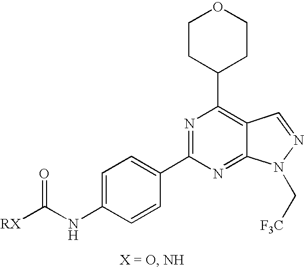

The 1-(4-(4-(3,6-dihydro-2H-pyran-4-yl)-1-(2,2,2-trifluoroethyl)-1H-pyrazolo[3,4-d]pyrimidin-6-yl)phenyl)urea and carbamate compounds were also made by a variant of the procedure of Scheme 1. Staring with (2,2,2-trifluoroethyl)hydrazine, 4-(4-(3,6-dihydro-2H-pyran-4-yl)-1-(2,2,2-trifluoroethyl)-1H-pyrazolo[3,4-d]pyrimidin-6-yl)aniline was made as shown in Scheme 14 using 4-aminophenylboronic acid, pinacol ester for the Suzuki coupling. The urea and carbamate compounds were made by activation with triphosgene (CCl₃OC(O)OCCl₃) or an equivalent followed by reaction with amine RNH₂ or alcohol ROH. Catalytic reduction of the 3,6-dihydro-2H-pyran-4-yl compound gave the tetrahydropyran shown in Scheme 14, if needed.

One of skill in the art will recognize that Schemes 1-14 can be adapted to produce the other 1H-pyrazolo[3,4-d]pyrimidine, purine, 7H-purin-8(9H)-one, 3H-[1,2,3]triazolo[4,5-d]pyrimidine, and thieno[3,2-d]pyrimidine compounds and pharmaceutically acceptable salts of thereof according to the present invention.

### EXAMPLES

The following Examples illustrate the synthesis of the 1H-pyrazolo[3,4-d]pyrimidine, purine, 7H-purin-8(9H)-one, 3H-[1,2,3]triazolo[4,5-d]pyrimidine, and thieno[3,2-d]pyrimidine compounds of the present invention.

### 3-[4-(3,6-dihydro-2H-pyran-4-yl)-1-phenyl-1H-pyrazolo[3,4-d]pyrimidin-6-yl]phenol (Scheme 11)

### Example 1

### Step 1

5-Amino-1-phenylpyrazole-4-carbonitrile was treated with 3-anisoyl chloride, 4-dimethylaminopyridine, and triethylamine in dichloromethane. Over the course of the reaction, additional anisoyl chloride was added. When the reaction was complete, it was concentrated to dryness under reduced pressure, dissolved in pyridine, and treated with water and then concentrated ammonium hydroxide. Following aqueous workup, crude 5-(3-methoxybenzamido)-1-phenylpyrazole-4-carbonitrile was provided as maroon foam.

### Step 2

5-(3-Methoxybenzamido)-1-phenylpyrazole-4-carbonitrile was treated with sodium hydroxide and 30% aqueous hydrogen peroxide in refluxing aqueous ethanol to give crude 6-(3-methoxyphenyl)-1-phenyl-1H-pyrazolo[3,4-d]pyrimidin-4-ol as a solid.

### Step 3

Heating of 6-(3-methoxyphenyl)-1-phenyl-1H-pyrazolo[3,4-d]pyrimidin-4-ol mmol) with phosphorous oxychloride in a sealed tube or microwave reactor gave 4-chloro-6-(3-methoxyphenyl)-1-phenyl-1H-pyrazolo[3,4-d]pyrimidine after concentration under reduced pressure. The crude solid was dissolved in dichloromethane and treated with boron tribromide to give 3-(4-bromo-1-phenyl-1H-pyrazolo[3,4-d]pyrimidin-6-yl)phenol after aqueous workup. The bromide was coupled to tributyl(3,6-dihydro-2H-pyran-4-yl)stannane [0.21 g, 0.57 mmol, prepared according to Kiely (U.S. Pat. No. 4,945,160) using palladium (II) bistriphenylphosphine dichloride in dimethylformamide and employing microwave irradiation to give 3-[4-(3,6-dihydro-2H-pyran-4-yl)-1-phenyl-1H-pyrazolo[3,4-d]pyrimidin-6-yl]phenol as pale yellow flakes, MS (ES⁺): 371.3 (M+H)⁺.

### 3-[1-phenyl-4-(tetrahydro-2H-pyran-4-yl)-1H-pyrazolo[3,4-d]pyrimidin-6-yl]phenol (Scheme 12)

### Example 2

3-[4-(3,6-dihydro-2H-pyran-4-yl)-1-phenyl-1H-pyrazolo[3,4-d]pyrimidin-6-yl]phenol was converted to 3-[1-phenyl-4-(tetrahydro-2H-pyran-4-yl)-1H-pyrazolo[3,4-d]pyrimidin-6-yl]phenol by hydrogenation in the presence of 10% palladium on carbon under 50 psi H₂ in 10% MeOH/CH₂Cl₂ for 22 hours. The mixture was filtered through Celite™ and concentrated to give the desired material, MS (ES⁺): 373.2 (M+H)⁺.

### N-{4-[1-(1-benzylpiperidin-4-yl)-4-(3,6-dihydro-2H-pyran-4-yl)-1H-pyrazolo[3,4-d]pyrimidin-6-yl]phenyl}acetamide (Scheme 6)

### Example 3

To a solution of 2,4,6-trichloro-pyrimidine-5-carbaldehyde (1.53 g, 7.19 mmol) in anhydrous ethanol (25 mL) at −78° C. was added (1-benzyl-piperidin-4-yl)-hydrazine hydrochloride (2 g, 7.19 mmol) and triethylamine (5.01 mL). After 30 min allow the reaction mixture to warm to 0° C. After 1 h warm to 25° C. Add ethyl acetate and extract with saturated aqueous sodium bicarbonate, water (2×) and brine. Dry over anhydrous magnesium sulfate. Concentrate in vacuo to give an oil. Add diethyl ether and remove precipitate by filtration. Concentrate mother liquor and add diethyl ether and remove precipitate by filtration. Add 2N HCl in diethyl ether to mother liquor and collect the precipitate. 1-(1-Benzyl-piperidin-4-yl)-4,6-dichloro-1H-pyrazolo[3,4-d]pyrimidine hydrochloride is obtained as a white solid is obtained. A mixture of this white solid (530 mg, 1.34 mmol), tributyl-(3,6-dihydro-2H-pyran-4-yl)-stannane (500 mg), PdCl₂(PPh₃)₂ (50 mg), diisopropylethyl amine (230 μL) in dimethylformamide (6 mL) is heated to 70° C. After 3 h at 70° C. and 18 h at 60° C.° the dimethylformamide is removed in vacuo. The residue is dissolved in dichloromethane and washed with saturated aqueous sodium bicarbonate. The organic phase is dried over anhydrous magnesium sulfate and concentrated in vacuo to give a dark oil. The oil is treated with 4-acetamidophenylboronic acid (72 mg, 0.402 mmol), Pd(PPh₃)₄ (5 mg) and 2M aqueous sodium carbonate (0.281 mL, 0.563 mmol) in dimethoxyethane (1 mL) and heated in a microwave at 175° C. for 15 min. The reaction mixture is purified by reverse phase HPLC (CH₃CN/H₂O/CF₃CO₂H) followed by silica gel chromatography (CH₂Cl₂/MeOH) to give the title compound as a trifluoroacetate salt (7.7 mg).

### 1-(4-(4-(3,6-dihydro-2H-pyran-4-yl)-1-(1-(pyridin-3-ylmethyl)piperidin-4-yl)-1H-pyrazolo[3,4-d]pyrimidin-6-yl)phenyl)-3-methylurea (Scheme 13)

### Example 4

### Step 1

6-Chloro-4-(3,6-dihydro-2H-pyran-4-yl)-1-(piperidin-4-yl)-1H-pyrazolo[3,4-d]pyrimidine was converted to 6-chloro-4-(3,6-dihydro-2H-pyran-4-yl)-1-(1-(pyridin-3-ylmethyl)piperidin-4-yl)-1H-pyrazolo[3,4-d]pyrimidine via reductive amination with pyridine-3-carboxaldehyde and sodium triacetoxyborohydride in tetrahydrofuran.

### Step 2

6-Chloro-4-(3,6-dihydro-2H-pyran-4-yl)-1-(1-(pyridin-3-ylmethyl)piperidin-4-yl)-1H-pyrazolo[3,4-d]pyrimidine was coupled with 4-(4,4,5,5-tetramethyl-1,3,2-dioxaborolan-2-yl)aniline using Suzuki conditions in the microwave. After aqueous work-up and HPLC purification, 4-(4-(3,6-dihydro-2H-pyran-4-yl)-1-(1-(pyridin-3-ylmethyl)piperidin-4-yl)-1H-pyrazolo[3,4-d]pyrimidin-6-yl)aniline trifluoroacetate was taken up in ethyl acetate and washed with a solution of sodium hydrogen carbonate. Drying of the organics, followed by concentration, provided the free base.

### Step 3

4-(4-(3,6-Dihydro-2H-pyran-4-yl)-1-(1-(pyridin-3-ylmethyl)piperidin-4-yl)-1H-pyrazolo[3,4-d]pyrimidin-6-yl)aniline was treated with triphosgene, followed by methylamine solution in tetrahydrofuran. After concentration and HPLC purification, 1-(4-(4-(3,6-dihydro-2H-pyran-4-yl)-1-(1-(pyridin-3-ylmethyl)piperidin-4-yl)-1H-pyrazolo[3,4-d]pyrimidin-6-yl)phenyl)-3-methylurea trifluoroacetate was obtained. MS (ES⁺): 525.1 (M+H)⁺

### 1-(4-{4-(3,6-dihydro-2H-pyran-4-yl)-1-[1-(pyridin-3-ylcarbonyl)piperidin-4-yl]-1H-pyrazolo[3,4-d]pyrimidin-6-yl}phenyl)-3-methylurea (Scheme 13)

### Example 5

### Step 1

4,6-dichloro-1H-pyrazolo[3,4-d]pyrimidine, prepared according to Robins (J Am Chem. Soc. Vol. 79, 1957, 6407-6415) was reacted with 3,4-dihydro-2H-pyran and catalytic p-toluenesulfonic acid in ethyl acetate at 70° C. The reaction mixture was diluted with saturated aqueous sodium hydrogen carbonate solution and extracted with ethyl acetate. After concentration of organic layers, the crude material was purified by flash chromatography to provide 4,6-dichloro-1-(tetrahydro-2H-pyran-2-yl)-1H-pyrazolo[3,4-d]pyrimidine.

### Step 2

4,6-Dichloro-1-(tetrahydro-2H-pyran-2-yl)-1H-pyrazolo[3,4-d]pyrimidine and tributyl(3,6-dihydro-2H-pyran-4-yl)stannane were coupled using palladium (II) bistriphenylphosphine dichloride in warm dimethylformamide. After flash chromatography, 6-chloro-4-(3,6-dihydro-2H-pyran-4-yl)-1-(tetrahydro-2H-pyran-2-yl)-1H-pyrazolo[3,4-d]pyrimidine was obtained, MS (ES⁺): 321.2 (M+H)⁺.

### Step 3

6-Chloro-4-(3,6-dihydro-2H-pyran-4-yl)-1-(tetrahydro-2H-pyran-2-yl)-1H-pyrazolo[3,4-d]pyrimidine in dichloromethane was treated with TFA. When the reaction was complete by TLC, the mixture was concentrated under reduced pressure and purified by flash chromatography to provide 6-chloro-4-(3,6-dihydro-2H-pyran-4-yl)-1H-pyrazolo[3,4-d]pyrimidine.

### Step 4

6-Chloro-4-(3,6-dihydro-2H-pyran-4-yl)-1H-pyrazolo[3,4-d]pyrimidine was reacted with tert-butyl 4-hydroxypiperidine-1-carboxylate under Mitsunobu conditions to give tert-butyl 4-(6-chloro-4-(3,6-dihydro-2H-pyran-4-yl)-1H-pyrazolo[3,4-d]pyrimidin-1-yl)piperidine-1-carboxylate.

### Step 5

Tert-butyl 4-(6-chloro-4-(3,6-dihydro-2H-pyran-4-yl)-1H-pyrazolo[3,4-d]pyrimidin-1-yl)piperidine-1-carboxylate in dichloromethane was treated with trifluoroacetic acid. After concentration, 6-chloro-4-(3,6-dihydro-2H-pyran-4-yl)-1-(piperidin-4-yl)-1H-pyrazolo[3,4-d]pyrimidine trifluoroacetate was obtained.

### Step 6

6-Chloro-4-(3,6-dihydro-2H-pyran-4-yl)-1-(piperidin-4-yl)-1H-pyrazolo[3,4-d]pyrimidine was converted to (4-(6-chloro-4-(3,6-dihydro-2H-pyran-4-yl)-1H-pyrazolo[3,4-d]pyrimidin-1-yl)piperidin-1-yl)(pyridin-3-yl)methanone via reaction with nicotinoyl chloride and triethylamine in dichloromethane.

### Step 7

(4-(6-Chloro-4-(3,6-dihydro-2H-pyran-4-yl)-1H-pyrazolo[3,4-d]pyrimidin-1-yl)piperidin-1-yl)(pyridin-3-yl)methanone was coupled with 4-(4,4,5,5-tetramethyl-1,3,2-dioxaborolan-2-yl)aniline using Suzuki conditions in the microwave. After aqueous work-up and HPLC purification, (4-(6-(4-aminophenyl)-4-(3,6-dihydro-2H-pyran-4-yl)-1H-pyrazolo[3,4-d]pyrimidin-1-yl)piperidin-1-yl)(pyridin-3-yl)methanone trifluoroacetate was obtained.

### Step 8

(4-(6-(4-Aminophenyl)-4-(3,6-dihydro-2H-pyran-4-yl)-1H-pyrazolo[3,4-d]pyrimidin-1-yl)piperidin-1-yl)(pyridin-3-yl)methanone was treated with triphosgene, followed by methylamine solution in tetrahydrofuran. After concentration and HPLC purification, 1-(4-{4-(3,6-dihydro-2H-pyran-4-yl)-1-[1-(pyridin-3-ylcarbonyl)piperidin-4-yl]-1H-pyrazolo[3,4-d]pyrimidin-6-yl}phenyl)-3-methylurea trifluoroacetate was obtained, MS (ES⁺): 539.4 (M+H)⁺.

### Preparation of 1-{4-[4-(3,6-dihydro-2H-pyran-4-yl)-1-(2,2,2-trifluoroethyl)-1H-pyrazolo[3,4-d]pyrimidin-6-yl]phenyl}-3-ureas (Scheme 14)

### Examples 6-10

### Step 1

5-Amino-1-(2,2,2-trifluoroethyl)-1H-pyrazole-4-carbonitrile was prepared by the condensation of trifluoroethylhydrazine (70% wt in water) and ethoxymethylenemalonitrile in ethanol with heating. At the end of the reaction, the mixture was concentrated under reduced pressure and subjected to an aqueous work-up with ethyl acetate. After drying and concentration, a yellow solid was obtained.

### Step 2

### 5-Amino-1-(2,2,2-trifluoroethyl)-1H-pyrazole-4-carbonitrile in acetonitrile and dichloromethane was treated with 4-nitrobenzoylchloride, triethylamine, and 4-dimethylaminopyridine. The mixture was heated, cooled, concentrated, and subjected to an aqueous work-up with ethyl acetate to give, after concentration, N-[4-cyano-1-(2,2,2-trifluoroethyl)-1H-pyrazol-5-yl]-4-nitrobenzamide.

### Step 3

N-[4-Cyano-1-(2,2,2-trifluoroethyl)-1H-pyrazol-5-yl]-4-nitrobenzamide was treated with sodium hydroxide and 30% aqueous hydrogen peroxide in refluxing aqueous ethanol to give 6-(4-nitrophenyl)-1-(2,2,2-trifluoroethyl)-1H-pyrazolo[3,4-d]pyrimidin-4-ol as a solid, after cooling, neutralization, and filtration.

### Step 4

Heating of 6-(4-nitrophenyl)-1-(2,2,2-trifluoroethyl)-1H-pyrazolo[3,4-d]pyrimidin-4-ol with phosphorous oxychloride) in a sealed tube gave 4-chloro-6-(4-nitrophenyl)-1-(2,2,2-trifluoroethyl)-1H-pyrazolo[3,4-d]pyrimidine after concentration under reduced pressure.

### Step 5

4-Chloro-6-(4-nitrophenyl)-1-(2,2,2-trifluoroethyl)-1H-pyrazolo[3,4-d]pyrimidine was hydrogenated under 1 atm hydrogen in the presence of 10% palladium on carbon and di-tert-butyldicarbonate in tetrahydrofuran. Following concentration of the mixture through Celite™ and concentration, tert-butyl {4-[4-chloro-1-(2,2,2-trifluoroethyl)-1H-pyrazolo[3,4-d]pyrimidin-6-yl]phenyl}carbamate was obtained.

### Step 6

Tert-butyl {4-[4-chloro-1-(2,2,2-trifluoroethyl)-1H-pyrazolo[3,4-d]pyrimidin-6-yl]phenyl}carbamate was coupled to tributyl(3,6-dihydro-2H-pyran-4-yl)stannane using palladium (II) bistriphenylphosphine dichloride in warm dimethylformamide to give, after flash chromatography, tert-butyl 4-(4-(3,6-dihydro-2H-pyran-4-yl)-1-(2,2,2-trifluoroethyl)-1H-pyrazolo[3,4-d]pyrimidin-6-yl)phenylcarbamate.

### Step 7

Tert-butyl 4-(4-(3,6-dihydro-2H-pyran-4-yl)-1-(2,2,2-trifluoroethyl)-1H-pyrazolo[3,4-d]pyrimidin-6-yl)phenylcarbamate in dichloromethane was treated with trifluoroacetic acid. Following concentration, the residue was taken up in ethyl acetate and washed with solutions of sodium hydrogen carbonate, sodium hydroxide, and brine. After concentration, 4-(4-(3,6-dihydro-2H-pyran-4-yl)-1-(2,2,2-trifluoroethyl)-1H-pyrazolo[3,4-d]pyrimidin-6-yl)aniline was obtained.

### Step 8

4-(4-(3,6-Dihydro-2H-pyran-4-yl)-1-(2,2,2-trifluoroethyl)-1H-pyrazolo[3,4-d]pyrimidin-6-yl)aniline in 1:1 dichloromethane/tetrahydrofuran was treated with triphosgene, followed by an excess of respective amine. After concentration and HPLC purification, the following compounds were obtained.

1-{4-[4-(3,6-dihydro-2H-pyran-4-yl)-1-(2,2,2-trifluoroethyl)-1H-pyrazolo[3,4-d]pyrimidin-6-yl]phenyl}-3-methylurea, Example 6 was obtained with methylamine. MS (ES⁺): 433.2 (M+H)⁺.

1-{4-[4-(3,6-dihydro-2H-pyran-4-yl)-1-(2,2,2-trifluoroethyl)-1H-pyrazolo[3,4-d]pyrimidin-6-yl]phenyl}-3-(2-fluoroethyl)urea, Example 7 was obtained with 2-fluoroethylamine, MS (ES⁺): 465.2 (M+H)⁺

1-{4-[4-(3,6-dihydro-2H-pyran-4-yl)-1-(2,2,2-trifluoroethyl)-1H-pyrazolo[3,4-d]pyrimidin-6-yl]phenyl}-3-pyridin-3-ylurea trifluoroacetate, Example 8 was obtained with 3-aminopyridine, MS (ES⁺): 496.2 (M+H)⁺.

### 1-(pyridin-3-yl)-3-(4-(4-(tetrahydro-2H-pyran-4-yl)-1-(2,2,2-trifluoroethyl)-1H-pyrazolo[3,4-d]pyrimidin-6-yl)phenyl)urea, (Scheme 14)

### Example 8a

Compound 8, 1-{4-[4-(3,6-dihydro-2H-pyran-4-yl)-1-(2,2,2-trifluoroethyl)-1H-pyrazolo[3,4-d]pyrimidin-6-yl]phenyl}-3-pyridin-3-ylurea trifluoroacetate , was catalytically reduced to provide the tetrahydropyran.

1-{4-[4-(3,6-dihydro-2H-pyran-4-yl)-1-(2,2,2-trifluoroethyl)-1H-pyrazolo[3,4-d]pyrimidin-6-yl]phenyl}-3-phenylurea, Example 9, was obtained with aniline, MS (ES⁺): 495.2 (M+H)⁺.

1-{4-[4-(3,6-dihydro-2H-pyran-4-yl)-1-(2,2,2-trifluoroethyl)-1H-pyrazolo[3,4-d]pyrimidin-6-yl]phenyl}-3-(4-(4-methylpiperazin-1-yl)phenyl)urea, Example 9a, and 1-{4-[4-(3,6-dihydro-2H-pyran-4-yl)-1-(2,2,2-trifluoroethyl)-1H-pyrazolo[3,4-d]pyrimidin-6-yl]phenyl}-3-(4-(4-methylpiperazine-1-carbonyl)phenyl)urea, Example 9b, were obtained analogously using the appropriately substituted analine.

1-{4-[4-(3,6-dihydro-2H-pyran-4-yl)-1-(2,2,2-trifluoroethyl)-1H-pyrazolo[3,4-d]pyrimidin-6-yl]phenyl}-3-ethylurea, Example 10 was obtained with ethylamine. MS (ES⁺): 447.2 (M+H)⁺.

### Preparation of 2-hydroxyethyl{4-[4-(3,6-dihydro-2H-pyran-4-yl)-1-(2,2,2-trifluoroethyl)-1H-pyrazolo[3,4-d]pyrimidin-6-yl]phenyl}carbamate (Scheme 14)

### Example 10a

4-(4-(3,6-Dihydro-2H-pyran-4-yl)-1-(2,2,2-trifluoroethyl)-1H-pyrazolo[3,4-d]pyrimidin-6-yl)aniline in 1:1 dichloromethane/tetrahydrofuran was treated with triphosgene, followed by an excess of ethylene glycol. After concentration and HPLC purification, the following title compound was obtained. MS (ES⁺): 464.7 (M+H)⁺.

### Preparation of 2-hydroxyethyl{4-[4-(tetrahydro-2H-pyran-4-yl)-1-(2,2,2-trifluoroethyl)-1H-pyrazolo[3,4-d]pyrimidin-6-yl]phenyl}carbamate (Scheme 14)

### Example 10b: Compound 10a

2-hydroxyethyl{4-[4-(3,6-dihydro-2H-pyran-4-yl)-1-(2,2,2-trifluoroethyl)-1H-pyrazolo[3,4-d]pyrimidin-6-yl]phenyl}carbamate, was catalytically reduced to provide the tetrahydropyran.

### Preparation of (3-[6-(3,6-Dihydro-2H-pyran-4-yl)-9H-purin-2-yl)]phenol

### Example 11

To a microwave processing tube dimethoxyethane (1.5 mL), saturated aqueous NaHCO₃ (1.5 mL), (Ph₃P)₄Pd (43 mg, 0.0369 mmol), 3-hydroxyphenyl boronic acid (153 mg, 1.11 mmol) and 2-chloro-6-(3,6-dihydro-1H-pyran-4-yl)-9H-purine (175 mg, 0.739 mmol) were added and the vessel was sealed. The mixture was heated to 175° C. for 30 minutes. The solvents were removed on a rotary evaporator and the crude compound was purified by HPLC to give the product as a TFA salt, brown solid (18 mg, 6% yield).

### Preparation of 3-[9-(1-benzylpiperidin-4-yl)-6-(3,6-dihydro-2H-pyran-4-yl)-9H-purin-2-yl]phenol, (Scheme 8)

### Example 12

A mixture of 3-[6-(3,6-dihydro-2H-pyran-4-yl)-9-piperidin-4-yl-9H-purin-2-yl]phenol (40 mg, 0.06 mmol), NaBH₃CN (31 mg, 0.49 mmol), and ZnCl₂ (33 mg, 0.75 mmol) and benzaldehyde (30 mg, 0.23 mmol) in methanol (1 mL) was stirred for 24 hours at room temperature. The mixture was filtered, dissolved in DMSO (1 mL) and purified by preparative HPLC to give the product (15 mg, 51% yield), MS (ESI) m/z=468.2.

### Preparation of 3-{6-(3,6-dihydro-2H-pyran-4-yl)-9-[1-(2-furylmethyl)piperidin-4-yl]-9H-purin-2-yl}phenol

### Example 13

3-[6-(3,6-dihydro-2H-pyran-4-yl)-9-piperidin-4-yl-9H-purin-2-yl]phenol (40 mg, 0.102 mmol), NaBH₃CN (31 mg, 0.147 mmol), and ZnCl₂ (33 mg, 0.75 mmol) was reacted according to the procedure used with Example 21 with 2-furylaldehyde (30 mg, 0.27 mmol) to give the product (14 mg, 31% yield), MS (ESI) m/z=458.2.

### Preparation of 3-[6-(3,6-dihydro-2H-pyran-4-yl)-9-{1-[(6-morpholin-4-ylpyridin-3-yl)methyl]piperidin-4-yl}-9H-purin-2-yl]phenol

### Example 14

3-[6-(3,6-dihydro-2H-pyran-4-yl)-9-piperidin-4-yl-9H-purin-2-yl]phenol (40 mg, 0.102 mmol), NaBH₃CN (31 mg, 0.147 mmol), and ZnCl₂ (33 mg, 0.75 mmol) was reacted according to the procedure used with Example 21 with 4-morpholinoyl-3-pyridylaldehyde (30 mg, 0.16 mmol) to give the product (22 mg, 38% yield), MS (ESI) m/z=554.

### Preparation of 3-{6-(3,6-dihydro-2H-pyran-4-yl)-9-[1-(1H-pyrrol-2-ylmethyl)piperidin-4-yl]-9H-purin-2-yl}phenol

### Example 15

3-[6-(3,6-dihydro-2H-pyran-4-yl)-9-piperidin-4-yl-9H-purin-2-yl]phenol (40 mg, 0.102 mmol), NaBH₃CN (31 mg, 0.147 mmol), and ZnCl₂ (33 mg, 0.75 mmol) was reacted according to the procedure used with Example 21 with 2-pyrrolaldehyde (30 mg, 0.31 mmol) to give the product (14 mg, 31% yield), MS (ESI) m/z=457.2.

### Preparation of 3-[6-(3,6-dihydro-2H-pyran-4-yl)-9-(1-methylpiperidin-4-yl)-9H-purin-2-yl]phenol

### Example 16

3-[6-(3,6-dihydro-2H-pyran-4-yl)-9-piperidin-4-yl-9H-purin-2-yl]phenol (40 mg, 0.102 mmol), NaBH₃CN (31 mg, 0.147 mmol), and ZnCl₂ (33 mg, 0.75 mmol) was reacted according to the procedure used with Example 21 with 37% aqueous formaldehyde solution (50 mg, 0.62 mmol) to give the product (13 mg, 33% yield), MS (ESI) m/z=392.2.

### Preparation of 3-{6-(3,6-dihydro-2H-pyran-4-yl)-9-[1-(1H-imidazol-5-ylmethyl)piperidin-4-yl]-9H-purin-2-yl}phenol

### Example 17

3-[6-(3,6-dihydro-2H-pyran-4-yl)-9-piperidin-4-yl-9H-purin-2-yl]phenol (40 mg, 0.102 mmol), NaBH₃CN (31 mg, 0.147 mmol), and ZnCl₂ (33 mg, 0.75 mmol) was reacted according to the procedure used with Example 21 with 5-imidazoylaldehyde (30 mg, 0.31 mmol) to give the product (23 mg, 50% yield), MS (ESI) m/z=458.2.

### Preparation of 3-{6-(3,6-dihydro-2H-pyran-4-yl)-9-[1-(4-methylbenzyl)piperidin-4-yl]-9H-purin-2-yl}phenol

### Example 18

3-[6-(3,6-dihydro-2H-pyran-4-yl)-9-piperidin-4-yl-9H-purin-2-yl]phenol (40 mg, 0.102 mmol), NaBH₃CN (31 mg, 0.147 mmol) and ZnCl₂ (33 mg, 0.75 mmol) was reacted according to the procedure used with Example 21 with p-tolylaldehyde (30 mg, 0.24 mmol) to give the product (20 mg, 42% yield), MS (ESI) m/z=482.2.

### Preparation of 3-[9-{1-[(6-bromopyridin-3-yl)methyl]piperidin-4-yl}-6-(3,6-dihydro-2H-pyran-4-yl)-9H-purin-2-yl]phenol

### Example 19

3-[6-(3,6-dihydro-2H-pyran-4-yl)-9-piperidin-4-yl-9H-purin-2-yl]phenol (40 mg, 0.102 mmol), NaBH₃CN (31 mg, 0.147 mmol) and ZnCl₂ (33 mg, 0.75 mmol) was reacted according to the procedure used with Example 21 with 6-bromo-pyridylaldehyde (30 mg, 0.16 mmol) to give the product (21 mg, 38% yield), MS (ESI) m/z=547.2.

### Preparation of 3-{9-[1-(3,4-difluorobenzyl)piperidin-4-yl]-6-(3,6-dihydro-2H-pyran-4-yl)-9H-purin-2-yl}phenol

### Example 20

3-[6-(3,6-dihydro-2H-pyran-4-yl)-9-piperidin-4-yl-9H-purin-2-yl]phenol (40 mg, 0.102 mmol), NaBH₃CN (31 mg, 0.147 mmol) and ZnCl₂ (33 mg, 0.75 mmol) was reacted according to the procedure used with Example 21 with 3,4difluorobenzlaldehyde (30 mg, 0.21 mmol) to give the product (32 mg, 63% yield), MS (ESI) m/z=504.2.

### Preparation of {3-[9-(1-Benzyl-piperidine-4-yl)-6-(3,6-dihydro-2H-pyran-4-yl]-phenyl}-methanol

### Example 21

A mixture of {3-[6-(3,6-Dihydro-2H-pyran-4-yl)-9-piperidin-4-yl-9H-purin-2-yl]-phenyl}methanol (40 mg, 0.102 mmol), NaBH(OAc)₃ (31 mg, 0.147 mmol), AcOH (8 μL, 0.133 mmol) and benzaldehyde (14 mg, 0.133 mmol) in 1 mL THF was stirred for 24 hours at room temperature. The mixture was filtered, dissolved in DMSO (1 mL) and purified by preparative HPLC to give the product (9.2 mg, 19% yield).

### Preparation of (3-{6-(3,6-Dihydro-2H-pyran-4-yl)-9-[1-(6-fluoro-pyridine-3-ylmethyl)-piperidine-4-yl]-9H-purine-2-yl}-phenyl-methanol

### Example 22

{3-[6-(3,6-Dihydro-2H-pyran-4-yl)-9-piperidin-4-yl-9H-purin-2-yl]-phenyl}methanol (40 mg, 0.102 mmol), NaBH(OAc)₃ (31 mg, 0.147 mmol), was reacted according to the procedure used with Example 21 with 2-fluoro-5-formyl pyridine (17 mg, 0.133 mmol) to give the product (10.4 mg, 19% yield).

### Preparation of (3-{6-(3,6-Dihydro-2H-pyran-4-yl)-9-[(1-pyridin-3-ylmethyl-piperidine-4-yl)-9H-purin-2-yl]-phenyl]}-methanol

### Example 23

{3-[6-(3,6-Dihydro-2H-pyran-4-yl)-9-piperidin-4-yl-9H-purin-2-yl]-phenyl}methanol (40 mg, 0.102 mmol), NaBH(OAc)₃ (31 mg, 0.147 mmol), was reacted according to the procedure used with Example 21 with 3-pyridine carboxaldehyde (14 mg, 0.133 mmol) to give the product (3.5 mg, 7% yield).

### Preparation of (3-{6-(3,6-Dihydro-2H-pyran-4-yl)-9-[(1-pyridin-2-ylmethyl-piperidine-4-yl)-9H-purin-2-yl]-phenyl]}-methanol

### Example 24

{3-[6-(3,6-Dihydro-2H-pyran-4-yl)-9-piperidin-4-yl-9H-purin-2-yl]-phenyl}methanol (40 mg, 0.102 mmol), NaBH(OAc)₃ (31 mg, 0.147 mmol), was reacted according to the procedure used with Example 21 with 2-pyridine carboxaldehyde (14 mg, 0.133 mmol) to give the product (14.2 mg, 29% yield).

### Preparation of (3-{6-(3,6-Dihydro-2H-pyran-4-yl)-9-[1-(6-bromo-pyridine-3-ylmethyl)-piperidine-4-yl]-9H-purine-2-yl}-phenyl-methanol

### Example 25

{3-[6-(3,6-Dihydro-2H-pyran-4-yl)-9-piperidin-4-yl-9H-purin-2-yl]-phenyl}methanol (40 mg, 0.102 mmol), NaBH(OAc)₃ (31 mg, 0.147 mmol), was reacted according to the procedure used with Example 21 with 6-bromopyridine-3-carboxaldehyde (25 mg, 0.133 mmol) to give the product (8.3 mg, 14% yield).

### 1-{4-[6-(3,6-dihydro-2H-pyran-4-yl)-9-piperidin-4-yl-9H-purin-2-yl]phenyl}-3-piperidin-4-ylurea

### Example 26

Benzyl-4-{[(4-{6-(3,6-dihydro-2H-pyran-4-yl)-9-[1-(1H-pyrrol-2-ylmethyl)piperidin-4-yl]-9H-purin-2-yl}phenyl)carbamoyl]amino}piperidine-1-carboxylate (60 mg, 0.08 mmol) was stirred in TFA (1 mL) for 24 hr. The solvents were removed in vacuo and the residue was dissolved in DMSO (1 mL) and purified by preparative HPLC to give the 1-{4-[6-(3,6-dihydro-2H-pyran-4-yl)-9-piperidin-4-yl-9H-purin-2-yl]phenyl}-3-piperidin-4-ylurea (5 mg, 12% yield), MS (ESI) m/z=503.

### Benzyl-4-{[(4-{6-(3,6-dihydro-2H-pyran-4-yl)-9-[1-(1H-pyrrol-2-ylmethyl)piperidin-4-yl]-9H-purin-2-yl}phenyl)carbamoyl]amino}piperidine-1-carboxylate

### Example 27

### Step 1

To a stirred solution of tert-butyl 4-[2-(4-aminophenyl)-6-(3,6-dihydro-2H-pyran-4-yl)-9H-purin-9-yl]piperidine-1-carboxylate (150 mg, 0.32 mmol) in CHCl₃ (2 mL) was added 4-isocyanato-piperidine-1-carboxylic acid benzyl ester (166 mg, 0.64 mmol, 2 eq) at 25° C. The reaction mixture was stirred for 30 min and TFA (0.5 mL) was added and stirring was continued for 1 hr. The mixture was filtered, dissolved in DMSO (1 mL) and purified by preparative HPLC to give the benzyl 4-{[(4-{6-(3,6-dihydro-2H-pyran-4-yl)-9-piperidin-4-yl]-9H-purin-2-yl}phenyl)carbamoyl]amino}piperidine-1-carboxylate (204 mg, 100% yield).

### Step 2

Benzyl 4-{[(4-{6-(3,6-dihydro-2H-pyran-4-yl)-9-piperidin-4-yl]-9H-purin-2-yl}phenyl)carbamoyl]amino}piperidine-1-carboxylate (60 mg, 0.32 mmol), NaBH₃CN (40 mg, 0.64 mmol), ZnCl₂ (44 mg, 0.31 mmol) was reacted according to the procedure used with Example 21 with 2-pyrrolocarbaldehyde (60 mg, 0.21 mmol) to give the product (60 mg, 26% yield), MS (ESI) m/z=716.4.

### Preparation of 5-(9-(1-benzylpiperidine-4-yl)-6-(3,6-dihydro-2H-pyran-4-yl)-9H-purin-2-yl)pyridine-3-ol

### Example 28

5-(6-(3,6-dihydro-2H-pyran-4-yl)-9-(piperidine-4-yl)-9H-purine-2-yl)pyridine-3-ol (40 mg, 0.106 mmol) was reacted according to the procedure used with Example 21 with benzaldehyde (15 mg, 0.138 mmol) to give the product (7.7 mg, 16% yield).

### Preparation of 5-(6-(3,6-dihydro-2H-pyran-4-yl)-9-(1-(pyridin-3-yl)methyl)piperidin-4-yl)-9H-purin-2-yl)pyridin-3-ol

### Example 29

5-(6-(3,6-Dihydro-2H-pyran-4-yl)-9-(piperidine-4-yl)-9H-purine-2-yl)pyridine-3-ol (40 mg, 0.106 mmol), was reacted according to the procedure used with Example 21 with 3-pyridine-carboxaldehyde (15 mg, 0.138 mmol) to give the product (26.3 mg, 53% yield).

### Preparation of 5-(9-(1-((6-bromopyridin-3-yl)methyl)piperidin-4-yl)-6-(3,6-dihydro-2H-pyran-4-yl)-9H-purine-2-yl)pyridin-3-ol

### Example 30

5-(6-(3,6-Dihydro-2H-pyran-4-yl)-9-(piperidine-4-yl)-9H-purine-2-yl)pyridine-3-ol (40 mg, 0.106 mmol), was reacted according to the procedure used with Example 21 with 6-bromopyridine-3-carboxaldehyde (26 mg, 0.138 mmol) to give the product (18.7 mg, 32% yield).

### Preparation of 5-(6-(3,6-dihydro-2H-pyran-4-yl)-9-(1-(pyridin-2-ylmethyl)piperidin-4-yl)-9H-purin-2-yl)pyridin-3-ol

### Example 31

5-(6-(3,6-Dihydro-2H-pyran-4-yl)-9-(piperidine-4-yl)-9H-purine-2-yl)pyridine-3-ol (40 mg, 0.106 mmol), was reacted according to the procedure used with Example 21 with 2-pyridine carboxaldehyde (15 mg, 0.138 mmol) to give the product (13.6 mg, 27% yield).

### Preparation of 5-(6-(3,6-dihydro-2H-pyran-4-yl)-9-(1-(6-methoxypyridine-3-ylmethyl)piperidin-4-yl)-9H-purin-2-yl)pyridin-3-ol

### Example 32

To the mixture of 5-(6-(3,6-dihydro-2H-pyran-4-yl)-9-(piperidine-4-yl)-9H-purine-2-yl)pyridine-3-ol (40 mg, 0.106 mmol), and 6-methoxy-3-pyridine carboxaldehyde (29 mg, 0.212 mmol) and 0.5 mL MeOH was added NaBH₃CN (13 mg, 0.212 mmol), ZnCl₂ (15 mg, 0.106 mmol) and 0.5 mL of MeOH . The mixture was stirred for 24 hours at room temperature and filtered, dissolved in DMSO (1 mL) and purified by preparative HPLC to give the product (19.2 mg, 22% yield).

### Preparation of 5-6-(3,6-dihydro-2H-pyran-4-yl)-(9-(1-((5-fluoro-1H-indole-3-yl)methyl)piperidine-4-yl)-9H-purine-2-yl)pyridin-3-ol

### Example 33

5-(6-(3,6-Dihydro-2H-pyran-4-yl)-9-(piperidine-4-yl)-9H-purine-2-yl)pyridine-3-ol (40 mg, 0.106 mmol), was reacted according to the procedure used with compound 32 with 6-methoxy-3-pyridine carboxaldehyde (35 mg, 0.212 mmol) to give the product (3.7 mg, 5% yield).

### Preparation of 5-(9-(1-((2-aminopyridin-3-yl)methyl)piperidine-4-yl)-6-(3,6-dihydro-2H-pyran-4-yl)-9H-purine-2-yl)pyridin-3-ol

### Example 34

5-(6-(3,6-Dihydro-2H-pyran-4-yl)-9-(piperidine-4-yl)-9H-purine-2-yl)pyridine-3-ol (40 mg, 0.106 mmol), was reacted according to the procedure used with compound 32 with 2-amino pyridine-3-carboxaldehyde (26 mg, 0.212 mmol) to give the product (19.9 mg, 23% yield).

### Preparation of 5-(9-(1-((5-bromopyridin-3-yl)methyl)piperidine-4-yl)-6-(3,6-dihydro-2H-pyran-4-yl)-9H-purine-2-yl)pyridin-3-ol

### Example 35

5-(6-(3,6-Dihydro-2H-pyran-4-yl)-9-(piperidine-4-yl)-9H-purine-2-yl)pyridine-3-ol (40 mg, 0.106 mmol), was reacted according to the procedure used with compound 32 with 5-bromo-3-formyl pyridine (39 mg, 0.212 mmol) to give the product (25.4 mg, 31% yield).

### Preparation of 5-(6-(3,6-dihydro-2H-pyran-4-yl)-9-(1-((2-methoxypyridin-3-yl)methyl)piperidine-4-yl)9H-purine-2-yl)pyridin-3-ol

### Example 36

5-(6-(3,6-Dihydro-2H-pyran-4-yl)-9-(piperidine-4-yl)-9H-purine-2-yl)pyridine-3-ol (40 mg, 0.106 mmol), was reacted according to the procedure used with compound 32 with 5-bromo-3-formyl pyridine (39 mg, 0.212 mmol) to give the product (25.4 mg, 31% yield).

### Preparation of 5-(9-(1-((6-chloropyridin-3-yl)methyl)piperidine-4-yl)-6-(3,6-dihydro-2H-pyran-4-yl)-9H-purine-2-yl)pyridin-3-ol

### Example 37

5-(6-(3,6-dihydro-2H-pyran-4-yl)-9-(piperidine-4-yl)-9H-purine-2-yl)pyridine-3-ol (40 mg, 0.106 mmol), was reacted according to the procedure used with compound 32 with 6-chloro pyridine-3-carboxaldehyde (30 mg, 0.212 mmol) to give the product (9.3 mg, 12% yield).

### Preparation of 6-(3,6-dihydro-2H-pyran-4-yl)-2-(3-hydroxyphenyl)-7,9-dihydro-8H-purin-8-one

### Example 38

### Step 1, Preparation of 4-dihydropyranoyl-5-nitro-6-amino-2-chloro-pyrimidine (Scheme 7)

In a three-necked flask under nitrogen were dissolved 5-nitro-6-amino-2,4-dichloropyrimidine (3.42 g, 16.52 mmol), tributyldihydropyranoylstanane (7.40 g, 19.8 mmol, 1.2 eq), and (Ph₃P)₂PdCl₂ (650 mg, 0.92 mmol, 0.05 eq) in anhydrous THF (35 ml). The reaction mixture was heated under stirring to 50° C. for 24 hrs. When the reaction was completed, the solvent was removed in vacuo and the crude product was purified by flash chromatography with CH₂Cl₂/EtOAc(10:1). The product was obtained as yellow solid (3.20 g, 70% yield), MS (ESI) m/z 257.0.

### Step 2, Preparation of 3-[4-amino-6-(3,6-dihydro-2H-pyran-4-yl)-5-nitropyrimidin-2-yl]phenol

In a three-necked flask was suspended 4-dihydropyranoyl-5-nitro-6-amino-2-chloro-pyrimidine (400 mg, 1.56 mmol), 3-hydroxyphenylboronic acid (323 mg, 2.34 mmol, 1.5 eq), DTBDF-PdCl₂ (101 mg, 0.16 mmol, 0.1 eq), K₃PO₄ (611 mg, 3.12 mmol, 2 eq) in anhydrous dioxane (10 mL). The reaction mixture was heated to reflux for 5 hrs. After the reaction was completed, the solvent was removed in vacuo and the crude product was purified by flash chromatography with CH₂Cl₂/MeOH/NH₃(25:1:0.1), and further purified by semi-prep-HPLC using ACN/water/TFA as mobile phase. After removal of solvents the product (200 mg, 30% yield) was obtained as yellow solid, MS (ESI) m/z 315.1.

### Step 3, Preparation of 4-dihydropyranoyl-5,6-diamino-2-(3-hydroxyphenyl)-pyrimidine

In a three-necked flask was suspended under nitrogen atmosphere 4-dihydropyranoyl-5-nitro-6-amino-2-(3-hydroxyphenyl)-pyrimidine (500 mg, 1.59 mmol), Raney™ nickel (370 mg) in methanol (50 mL). To the stirring reaction mixture was added slowly hydrazine (1.5 mL, 47 mmol, 24 eq) and the stirring was continued for 0.5 hrs to drive the reduction to completion. The reaction mixture was filtered over Celite™ and the filtrate was evaporated and purified by flash chromatography using CH₂Cl₂/MeOH/NH₃(10:1:0.1) to obtain the product (460 mg, 100% yield) as off-white solid, MS (ESI) m/z 285.2.

### Step 4

In a three-necked flask was stirred 4-dihydropyranoyl-5,6-diamino-2-(3-hydroxyphenyl)-pyrimidine (340 mg, 1.20 mmol), K₂CO₃ (331 mg, 2.4 mmol, 2 eq) in anhydrous acetonitrile and phosgene (20% in toluene) (1.26 ml, 2.4 mmol, 2 eq) was added slowly to the suspension at 0° C. After stirring for another 2 hrs at 25° C. solvent was removed in vacuo and the residue was purified by semi-prep-HPLC using ACN/water/TFA as mobile phase. After removal of the solvent, the product (130 mg, 35% yield) was obtained as white solid, MS (ESI) m/z 323.1.

### Preparation of 6-(3,6-dihydro-2H-pyran-4-yl)-2-[3-(hydroxymethyl)phenyl]-7,9-dihydro-8H-purin-8-one

### Example 39

### Step 1, Preparation of 4-dihydropyranoyl-5-nitro-6-amino-2-(3-hydroxymethylphenyl)-pyrimidine

In a three-necked flask was suspended 4-dihydropyranoyl-5-nitro-6-amino-2-chloro-pyrimidine (1.5 g, 5.86 mmol), 3-hydroxymethylphenylboronic acid (1.34 g, 8.79 mmol, 1.5 eq), DTBDF-PdCl₂ (200 mg, 0.31 mmol, 0.05 eq), K₃PO₄ (2.31 g, 11.72 mmol, 2 eq) in anhydrous dioxane (45 mL). The reaction mixture was heated under stirring to reflux for 5 hrs. After the reaction was completed, the solvent was removed in vacuo and the crude product was purified by flash chromatography with CH₂Cl₂/MeOH/NH₃(25:1:0.1). After removal of solvents the product (600 mg, 30% yield) was obtained as yellow solid. Additionally, starting material (700 mg) was isolated, MS (ESI) m/z 329.1.

### Step 2, Preparation of 4-dihydropyranoyl-5,6-diamino-2-(3-hydroxymethylphenyl)-pyrimidine

In a three-necked flask was suspended under nitrogen atmosphere 4-dihydropyranoyl-5-nitro-6-amino-2-(3-hydroxymethylphenyl)-pyrimidine (600 mg, 1.81 mmol), Raney™ nickel (1.8 g) in methanol (100 mL). To the stirring reaction mixture was added slowly hydrazine (1.5 mL, 47 mmol, 24 eq) and the stirring was continued for 0.5 hrs to drive the reduction to completion. The reaction mixture was filtered over Celite™ and the filtrate was evaporated to obtain the product (400 mg, 73% yield) as off-white solid.

### Step 3

In a three-necked flask was stirred 4-dihydropyranoyl-5,6-diamino-2-(3-hydroxymethylphenyl)-pyrimidine (400 mg, 1.33 mmol), K₂CO₃ (367 mg, 2.7 mmol, 2 eq) in anhydrous acetonitrile and triphosgene (197 mg, 0.66 mmol, 0.5 eq) was added to the suspension ad 0° C. After stirring for another 2 hrs at 25° C. solvent was removed in vacuo and the residue was purified by semi-prep-HPLC using ACN/water/TFA as mobile phase. After removal of the solvent, the product (155 mg, 36% yield) was obtained as white solid.

### Preparation of 3-[6-(3,6-dihydro-2H-pyran-4-yl)-9-piperidin-4-yl-9H-purin-2-yl]phenol

### Example 40

To a microwave processing tube dimethoxyethane (2 mL), aqueous Na₂CO₃ (2 molar) (0.5 mL, 1 mmol), (Ph₃P)₄Pd (66 mg, 0.05 mmol), 3-hydroxyphenyl-boronic acid (108 mg, 0.78 mmol, 1.6 eq) and tert-butyl 4-(2-chloro-6-(3,6-dihydro-2H-pyran-4-yl)-9H-purin-9-yl)piperidine-1-carboxylate (200 mg, 0.48 mmol) were added and the vessel was sealed. The mixture was heated to 130° C. for 30 minutes. The solvents were removed on a rotary evaporator and the crude compound was purified by silica gel chromatography (50% hexane/EtOAc) to give the product as a white solid (200 mg, 87% yield).

To the white solid and 2 ml of CH₂Cl₂ was added TFA (0.361 mL, 4.68 mmol). The mixture was stirred at room temp. for 3 hrs. and made basic with 1N NaOH then extracted with CH₂Cl₂. The solvent was evaporated and purified by HPLC to give the product as a TFA salt (40 mg, 15% yield), MS (ESI) m/z=378.2.

### 1-(4-{6-(3,6-dihydro-2H-pyran-4-yl)-9-[1-(1H-pyrrol-2-ylmethyl)piperidin-4-yl]-9H-purin-2-yl}phenyl)-3-methylurea

### Example 41

### Step 1, Preparation of tert-butyl 4-[2-(4-aminophenyl)-6-(3,6-dihydro-2H-pyran-4-yl)-9H-purin-9-yl]piperidine-1-carboxylate

To a microwave processing tube dimethoxyethane (2 mL), aqueous Na₂CO₃ (2 molar) (1.2 mL, 2.4 mmol, 2 eq), (Ph₃P)₄Pd (122 mg, 0.1 mmol), 4-aminophenyl boronic acid pinacol ester (389 mg, 1.78 mmol, 1.5 eq) and tert-butyl 4-(2-chloro-6-(3,6-dihydro-2H-pyran-4-yl)-9H-purin-9-yl)piperidine-1-carboxylate (500 mg, 1.19 mmol) were added and the vessel was sealed. The mixture was heated to 120° C. for 30 minutes and to 175° C. for 15 minutes. The solvents were removed on a rotary evaporator and the crude compound was purified by silica gel chromatography (CH₂Cl₂/MeOH/NH₃) to give the product as a white solid (440 mg, 78% yield), MS (ESI) m/z 477.4.

### Step 2

To a stirred solution of triphosgene (62 mg, 0.21 mmol) in CHCl₃ (1 mL) was added tert-butyl 4-[2-(4-aminophenyl)-6-(3,6-dihydro-2H-pyran-4-yl)-9H-purin-9-yl]piperidine-1-carboxylate (150 mg, 0.32 mmol) at 0° C. The reaction mixture was stirred for 30 min and NH₂Me (2 molar solution in THF) and NEt₃ (133 ml, 0.96 mmol, 3 eq) as added also at 0° C. for 3 hrs. The solvents were removed on a rotary evaporator and the crude compound was dissolved in TFA/CHCl₃ (20%) stirred for 12 hrs at 25° C. After the reaction was completed the solvent was removed in vacuo and the residue dissolved in aqueous NaOH (5 mL) and extracted with CHCl3 (3×10 mL). The combined organic layers were dried over MgSO₄. After removal of the drying agents by filtration and evaporation of solvents an off-white solid was obtained.

### Step 3

The crude material, NaBH₃CN (40 mg, 0.64 mmol), ZnCl₂ (44 mg, 0.31 mmol) and 2-pyrrolocarbaldehyde (60 mg, 0.64 mmol) in methanol (2 mL) was stirred for 24 hours at room temperature. The mixture was filtered, dissolved in DMSO (1 mL) and purified by preparative HPLC to give the product (46 mg, 28% yield), MS (ESI) m/z=514.

### Preparation of 4-[6-(3,6-Dihydro-2H-pyran-4-yl)-2-(3-hydroxymethyl-phenyl)-purin-9-yl]-piperidine-1-carboxylic acid tert. butyl ester

### Example 42

### Step 1, Preparation of tert-butyl 4-(2,6-dichloro-9-H-purin-9-yl)piperidine-1-carboxylate

To the t-butyl-4-hydroxy-1-piperidine (2.13 g, 10.58 mmol) and PPh₃ (2.78 g, 10.58 mmol) in THF (30 mL) was added 2,6-dichloro-9H-purine (1.0 g, 5.29 mmol) stirred the solution for 30 minutes. and added DIAD at 0° C. The mixture was stirred overnight, concentrated and purify by silica gel chromatography (10% MeOH in EtOAc-Hex) to give the product as a white solid (920 mg, 47% yield)

### Step 2, Preparation of tert-butyl 4-(2-chloro-6-(3,6-dihydro-2H-pyran-4-yl)-9H-purin-9-yl)piperidine-1-carboxylate

To the tert-butyl 4-(2,6-dichloro-9-H-purin-9-yl)piperidine-1-carboxylate (200 mg, 0.537 mmol) in 2 mL of DMF was added tributyl(3,6-dihydro-2H-pyran-4-yl)stannane (240 mg. 0.644 mmol), (Ph₃P)₂PdCl₂ (38 mg, 0.054 mmol), and CuI (10 mg, 0.054 mmol). The mixture was heated at 90° C. overnight, concentrated and purify by silica gel chromatography (10% MeOH in CHCl₃) to give the product as a tan solid (130 mg, 58% yield).

### Step 3

To a microwave processing tube dimethoxyethane (8 mL), saturated aqueous NaHCO₃ (3 mL), (Ph₃P)₄Pd (113 mg, 0.098 mmol), 3-(hydroxymethyl)phenyl-boronic acid (445 mg, 2.93 mmol) and tert-butyl 4-(2-chloro-6-(3,6-dihydro-2H-pyran-4-yl)-9H-purin-9-yl)piperidine-1-carboxylate (820 mg, 1.96 mmol) were added and the vessel was sealed. The mixture was heated to 130° C. for 30 minutes. The solvents were removed on a rotary evaporator and the crude compound was purified by silica gel chromatography (10% MeOH in CHCl3) to give the product as a white solid (280 mg, 57% yield) of white solid.

### Preparation of {3-[6-(3,6-Dihydro-2H-pyran-4-yl)-9-piperidin-4-yl-9H-purin-2-yl]-phenyl}methanol

### Example 43

To the 4-[6-(3,6-dihydro-2H-pyran-4-yl)-2-(3-hydroxymethyl-phenyl)-purin-9-yl]-piperidine-1-carboxylic acid tert. butyl ester (230 mg, 0.468 mmol) and 2 ml of CH₂Cl₂ was added TFA (0.361 mL, 4.68 mmol). The mixture was stirred at room temp. for 3 hrs. and made basic with 1N NaOH then extracted with CH₂Cl₂. The solvent was evaporated and purified by HPLC to give the product as a TFA salt (58 mg, 15% yield).

### Preparation of 5-(6-(3,6-dihydro-2H-pyran-4-yl)-9-(piperidine-4-yl)-9-(piperidine-4-yl)-9H-purin-2-yl)pyridine-3-ol

### Example 44

### Step 1, Preparation of tert-butyl 4-(6-(3,6-dihydro-2H-pyran-4-yl)-2-(3-(methoxy)phenyl)-9H-purin-9-yl)piperidine-1-carboxylate

To a microwave processing tube dimethoxyethane (5 mL), 2M Na₂CO₃ (1.43 mL), (Ph₃P)₄Pd (165 mg, 0.143 mmol), 3-(methoxyethoxy)-5-(4,4,5,5-tetramethyl-1,3,2-dioxaborolan-2yl)pyridine (568 mg, 2.14 mmol) and tert-butyl 4-(2-chloro-6-(3,6-dihydro-2H-pyran-4-yl)-9H-purin-9-yl)piperidine-1-carboxylate (600 mg, 1.43 mmol) were added and the vessel was sealed. The mixture was heated to 175° C. for 15 minutes. The solvents were removed on a rotary evaporator and the crude compound was purified by silica gel chromatography EtOAc:Hex (1:1) to give the product as a white solid (190 mg, 25% yield) of tan solid.

### Step 2

To the tert-butyl 4-(6-(3,6-dihydro-2H-pyran-4-yl)-2-(3-(methoxy)phenyl)-9H-purin-9-yl)piperidine-1-carboxylate (300 mg, 0.575 mmol) and 5 ml of CH₂Cl₂ was added HCl (1.5 mL) was heated under reflux for 1 hr. The mixture was made basic with 1N NaOH and purified by HPLC to give the product as a tan solid (120 mg, 55% yield).

### Preparation of (3-[6-(3,6-dihydro-2H-pyran-4-yl)-9-(2-piperidin-1-yl)ethyl)-9-H-purin-2-yl)phenyl)methanol

### Example 45

### Step 1, Preparation of 2,6-dichloro-9-(2-(piperidine-1-yl)ethyl)-9-H-purine

2,6-Dichloro-9H-purine (2.5 g, 13.23 mmol) was reacted according to procedure of step 1 of compound 42 with 2-(piperidine-1-yl)ethanol (3.42 g, 26.46 mmol) to give the product as a yellow oil (1.8 g, 45% yield).

### Step 2, Preparation of 2-chloro-6-(3,6-dihydro-2H-pyran-4-yl)-9-(2-piperidin-1-yl)ethyl)-9-H-purine

2-Chloro-9-(2-(piperidine-1-yl)ethyl)-9-H-purine (1.2 g, 4 mmol) was reacted according to the procedure of step 2 of compound 42 with tributyl (3,6-dihydro-2H-pyran-4-yl)stannane (1.9 g, 4.8 mmol) to give the product as a tan solid (482 mg, 35%).

### Step 3

2-Chloro-6-(3,6-dihydro-2H-pyran-4-yl)-9-(2-piperidin-1-yl)ethyl)-9-H-purine (150 mg, 0.431 mmol) was reacted according to the procedure of step 3 of compound 42 with 3-(hydroxymethyl)phenyl-boronic acid (98 mg, 0.647 mmol) to give the product as a tan solid (145 mg, 63% yield).

### Preparation of (3-[6-(3,6-dihydro-2H-pyran-4-yl)-9-(2-piperidin-1-yl)ethyl)-9-H-purin-2-yl)phenyl)methanol

### Example 46

2-Chloro-6-(3,6-dihydro-2H-pyran-4-yl)-9-(2-piperidin-1-yl)ethyl)-9-H-purine (100 mg, 0.288 mmol) was reacted according to the procedure of step 3 of compound 42 with 3-hydroxy phenyl-boronic acid (60 mg, 0.431 mmol) to give the product as a tan solid (58 mg, 50% yield).

### 3-[3-(1-benzylpiperidin-4-yl)-7-(3,6-dihydro-2H-pyran-4-yl)-3H-[1,2,3]triazolo[4,5-d]pyrimidin-5-yl]phenol

### Example 47

### Step 1, Preparation of 4-(2,6-Dichloro-5-nitro-pyrimidin-4-ylamino)-piperidine-1-carboxylic Acid Tert-Butyl Ester

To a solution of 2,4,6-trichloronitropyrimidine (300 mg, 1.32 mmol) in CH₂Cl₂ (5 mL) at 0° C. was added a solution of 4-amino-1-BOC-piperidine (243 mg, 0.9 eq) and NEt₃ (121 mg, 1.2 mmol, 0.9 eq) in THF (3 mL). The reaction mixture was stirred for another 1 hr at 25° C. to drive the reaction to completion. For purification silica gel (2 g) was added to the mixture and the solvent was removed so that product was absorbed on the silica gel. The silica gel plug was placed on a column and the product purified by flash chromatography with CH₂Cl₂ to give after removal of solvent the product as yellow solid (380 mg, 80% yield), MS (ESI) m/z 393.2.

### Step 2, Preparation of Tert-Butyl 4-{[2-chloro-6-(3,6-dihydro-2H-pyran-4-yl)-5-nitropyrimidin-4-yl]amino}piperidine-1-carboxylate

In a three-necked flask under nitrogen was dissolved 4-(2,6-dichloro-5-nitro-pyrimidin-4-ylamino)-piperidine-1-carboxylic acid tert-butyl ester (350 mg, 0.89 mmol), tributyldihydropyranoylstanane (399 mg, 1.07 mmol, 1.2 eq), and (Ph₃P)₂PdCl₂ (35 mg, 0.05 mmol, 0.05 eq) in anhydrous THF (2 ml). The reaction mixture was heated under stirring to 50° C. for 24 hrs. When the reaction was completed, the solvent was removed in vacuo and the crude product was purified by flash chromatography with CH₂Cl EtOAc(10:1). The product was obtained as yellow solid (140 mg, 35% yield), MS (ESI) m/z 440.3.

### Preparation of Tert-Butyl 4-[5-chloro-7-(3,6-dihydro-2H-pyran-4-yl)-3H-[1,2,3]triazolo[4,5-d]pyrimidin-3-yl]piperidine-1-carboxylate

### Step 3

In a three-necked flask was suspended under a nitrogen atmosphere tert-butyl 4-{[2-chloro-6-(3,6-dihydro-2H-pyran-4-yl)-5-nitropyrimidin-4-yl]amino}piperidine-1-carboxylate (900 mg, 2.05 mmol), Raney™ nickel (2 g) in methanol (90 mL). To the reaction mixture while stirring was added slowly hydrazine (1.5 mL, 47 mmol, 23 eq) and the stirring was continued for 0.5 hrs to drive the reduction to completion. The reaction mixture was filtered over Celite™. The filtrate was evaporated and used without crude.

### Step 4

To a stirred solution of the crude material (2.05 mmol) in acetic acid/water (1:1) (14 mL) at 0° C. was added aqueous 0.5 N NaNO₂-solution (7.5 mL, 3.75 mmol, 1.8 eq) and the reaction mixture was allowed to stir for 2 hrs. The solid was collected by filtration and dried at 45° C. in vacuo to give the product (500 mg, 57% yield) as white solid, MS (ESI) m/z 421.

### Step 5

In a microwave vial was suspended tert-butyl 4-[5-chloro-7-(3,6-dihydro-2H-pyran-4-yl)-3H-[1,2,3]triazolo[4,5-d]pyrimidin-3-yl]piperidine-1-carboxylate, (500 mg, 1.19 mmol), 3-hydroxymethylphenylboronic acid (331 mg, 2.4 mmol, 2 eq), (Ph₃P)₄Pd (137 mg, 0.01 mmol, 0.1 eq), and saturated aqueous Na₂CO₃ (2N, 1.2 mL, 2.4 mmol, 2 eq) in DME (4 mL). The reaction mixture was heated under stirring to 175° C. for 15 minutes. After the reaction was completed, the solvent was removed in vacuo and the crude product was purified by flash chromatography with CH₂Cl₂/MeOH/NH₃(10:1:0.1). to give an off white solid.

### Step 6

The dissolved in CHCl₃/TFA (1:1, 5 mL) and stirred for 2 hrs at 25° C. After the reaction was completed, the solvents were removed in vacuo and the crude product was purified by semi-prep-HPLC using ACN/water/TFA as mobile phase. After solvent removal a white solid was obtained, which was dissolved in methanol (2 mL) and benzaldehyde (126 mg, 1.19 mmol, 1 eq), NaBH₃CN (75 mg, 1.19 mmol, 1 eq) and ZnCl₂ (74 mg, 0.6 mmol, 0.5 eq) was added. The suspension was stirred for 24 hrs and the solvents were removed in vacuo. The crude product was purified by semi-prep-HPLC using ACN/water/TFA as mobile phase. After solvent removal, the product was obtained as a white solid (10 mg, 1% yield), MS (ESI) m/z 469.1.

### Preparation of 3-[7-(3,6-dihydro-2H-pyran-4-yl)-3H-[1,2,3]triazolo[4,5-d]pyrimidin-5-yl]phenol

### Example 48

To a stirred solution of 4-dihydropyranoyl-5,6-diamino-2-(3-hydroxyphenyl)-pyrimidine (150 mg, 0.53 mmol) in acetic acid/water (1:1) (4 mL) at 0° C. was added aqueous 0.5 N NaNO₂-solution (2 mL, 1 mmol, 1.9 eq) and the reaction mixture was allowed to stir for 2 hrs. The solid was collected by filtration and than further purified by semi-prep-HPLC using ACN/water/TFA as mobile phase. After removal of the solvent, the product (15 mg, 10% yield) was obtained as white solid, MS (ESI) m/z 296.1.

### Preparation of {3-[7-(3,6-dihydro-2H-pyran-4-yl)-3H-[1,2,3]triazolo[4,5-d]pyrimidin-5-yl]phenyl}methanol

### Example 49

### Preparation of 2-Chloro-6-morpholin-4-yl-8-aza-purine

### Step 1

In a three-necked flask was suspended under a nitrogen atmosphere 4-dihydropyranoyl-5-nitro-6-amino-2-chloro-pyrimidine (500 mg, 1.95 mmol), Raney™ nickel (0.8 g) in methanol (50 mL). To the stirring reaction mixture was added slowly hydrazine (0.4 mL, 12.5 mmol, 6.4 eq) and the stirring was continued for 0.5 hrs to drive the reduction to completion. The reaction mixture was filtered over Celite™ and the filtrate was evaporated to obtain the product (262 mg, 59% yield) as off-white solid.

### Step 2

To a stirred solution of 4-dihydropyranoyl-5,6-diamino-2-chloro-pyrimidine (225 mg, 0.55 mmol) in acetic acid/water (1:1) (4 mL) at 0° C. was added aqueous 0.5 N NaNO₂-solution (2 mL, 1 mmol, 1.9 eq) and the reaction mixture was allowed to stir for 2 hrs. The solid was collected by filtration and dried at 45° C. in vacuo to give the product (110 mg, 84% yield) as white solid, MS (ESI) m/z 296.1.

### Step 3

In a microwave vial was suspended 2-cloro-6-morpholin-4-yl-8-aza-purine (110 mg, 0.46 mmol), 3-hydroxymethylphenylboronic acid (140 mg, 0.92 mmol, 2 eq), (Ph₃P)₄Pd (27 mg, 0.2 mmol, 0.05 eq), and saturated aqueous NaHCO₃ (0.5 mL) in DME (3 mL). The reaction mixture was heated under stirring to 175° C. for 15 minutes. After the reaction was completed, the solvent was removed in vacuo and the crude product was purified by semi-prep-HPLC using ACN/water/TFA as mobile phase. After removal of solvents, the product (60 mg, 42% yield) was obtained as off-white solid, MS (ESI) m/z 309.1.

### Preparation of 5-[7-(3,6-dihydro-2H-pyran-4-yl)-3H-[1,2,3]triazolo[4,5-d]pyrimidin-5-yl]pyridin-3-ol

### Example 50

In a microwave vial was suspended 2-cloro-6-morpholin-4-yl-8-aza-purine (200 mg, 0.84 mmol), 5-methoxymethyl-3-pyridyl boronic acid pinacol ester (267 mg, 1.10 mmol, 1.2 eq), (Ph₃P)₄Pd (54 mg, 0.4 mmol, 0.05 eq), and saturated aqueous NaHCO₃ (1 mL) in DME (4 mL). The reaction mixture was heated under stirring to 175° C. for 15 minutes. After the reaction was completed, the solvent was removed in vacuo and the crude product was purified by flash chromatography with CH₂Cl₂/MeOH/NH₃(15:1:0.1). After removal of solvents the crude methoxymethyl ether-protected product was refluxed in methanol (5 mL) and conc. HCl (1 mL) for 1 hr and the crude product was purified by semi-prep-HPLC using ACN/water/TFA as mobile phase. After removal of solvents, the product (35 mg, 14% yield) was obtained as off-white solid, MS (ESI) m/z 296.2.

### Thienopyrimidine Experimentals for Examples 51-65

### Preparation of Thieno[3,2-d]pyrimidine-2,4-diol

### Step 1

A 50 mg (0.35 mmol) portion of 3-amino-thiophene-2-carboxamide is dissolved in 3 mL dioxane. 52 mg (0.175 mmol, 0.5 eq) of triphosgene is added and the mixture is heated at 80 C. After 30 min, TLC and LC/MS indicate that the reaction is complete. The mixture is cooled down to room temperature and the resulting white precipitate is collected and washed with diethyl ether. Yield: 46 mg (0.27 mmol, 78%).

### Alternative Synthesis

A 50 mg (0.35 mmol) portion of 3-amino-2-aminocarbonyl-thiophene is covered with urea. The urea is heated until molten. After 5 minutes of heating, the molten mixture is poured into 1 N NaOH (2 mL). Acetic acid is added to neutralize the mixture (pH 7) and the resulting white precipitate is collected. Yield: 39 mg (0.23 mmol, 66%).

### Large Scale Synthesis

A 500 mg (3.5 mmol) portion of 3-amino-thiophene-2-carboxamide is dissolved in 30 mL dioxane. 520 mg (1.75 mmol, 0.5 eq) triphosgene is added and the mixture is heated at 80° C. for 1 h. The mixture is concentrated and triturated from hot methanol. The resulting solids are collected. The filtrate is concentrated and triturated again from hot methanol. The 2 crops of solids are combined to give 432 mg of the title compound (2.57 mmol, 73%).

### Preparation of 2,4-Dichloro-thieno[3,2-d]pyrimidine

### Step 1

A 85 mg portion of thieno[3,2-d]pyrimidine-2,4-diol (0.5 mmol) is suspended in 1.25 mL POCl₃. The mixture is heated at 100° C. overnight. POCl₃ is removed under reduced pressure. The mixture is dissolved in dichloromethane and quenched with ice. The product is collected by extraction with dichloromethane (2×). The combined organic layers are dried over MgSO4 and concentrated to give the title compound in quantitative yield that was used in the next step without further purification.

### Large Scale Synthesis

A 432 mg portion of thieno[3,2-d]pyrimidine-2,4-diol (2.57 mmol) is suspended in 6.25 mL POCl₃. The mixture is heated at 100° C. overnight, POCl₃ is removed under reduced pressure, and toluene added to remove any residue by azeotrope distillation. The mixture is dissolved in dichloromethane and quenched with ice. The product is collected by extraction with dichloromethane (2×). The combined organic layers are dried over MgSO₄ and concentrated to give the title compound in quantitative yield that was used in the next step without further purification.

### Step 3, Preparation of 2-Chloro-4-(3,6-dihydro-2H-pyran-4-yl)-thieno[3,2-d]pyrimidine

A 464 mg (2.26 mmol) portion of 2,4-dichloro-thieno[3,2-d]pyrimidine was dissolved in 14 mL anhydrous THF. 1,016 mg (2.7 mmol, 1.2 eq) of tributyl-(3,6-dihydro-2H-pyran-4-yl)-stannane was added and nitrogen was bubbled through the mixture for 5 min to degas. Pd(PPh₃)₂Cl₂ (159 mg, 10 mol %) was added and the mixture was heated at reflux. After 4 h, TLC indicated the reaction to be complete. The mixture was concentrated and purified by column chromatography (0-25% EtOAc in hexanes) to give 425 mg (1.68 mmol, 74%) of the title compound.

### Step 4, General Procedure for Suzuki Reaction of the thieno[3,2-d]pyrimidine Examples

A 30 mg (0.12 mmol) of 2-chloro-4-(3,6-dihydro-2H-pyran-4-yl)-thieno[3,2-d]pyrimidine and 0.2 mmol of the aryl boronic acid or boronic acid pinacol ester were dissolved in 1 mL toluene and 0.6 mL ethanol. 0.24 mL of a 2M solution of aqueous Na₂CO₃ was added and nitrogen was bubbled through the mixture for 5 min to degas. A 14 mg (10 mol %) portion of Pd(PPh₃)₄ was added and the mixture was heated under microwave irradiation for 60 min at 120° C. The solvents were removed under a stream of nitrogen, the residue was dissolved in 2 mL DMSO, filtered and purified by HPLC (NH₃ buffers).

### Step 5, General Procedure for Formation of Urea of the thieno[3,2-d]pyrimidine Examples

A 30 mg (0.12 mmol) portion of 2-chloro-4-(3,6-dihydro-2H-pyran-4-yl)-thieno[3,2-d]pyrimidine was reacted with 4-aminophenylboronic acid, pinacol ester as above. Upon completion of the Suzuki reaction, the mixture was diluted with EtOAc and filtered over Celite™. The mixture was washed 2× with brine and the organic phase was dried over MgSO₄ and concentrated. The resulting product was dissolved in 1 mL dichloromethane and 0.065 mL NEt₃ was added. The resulting solution was added drop wise to a solution of triphosgene (18 mg, 0.06 mmol, 0.5 eq) in 1 mL dichloromethane. After stirring at room temperature for 10 minutes, the mixture was added to a solution of 1 mmol of amine in dichloromethane (for aniline, 2-aminopyridine and 3-aminopyridine) or to a solution of 2 mmol of amine in THF (for methyl amine or ethyl amine) or to a solution of 1 mmol of the amine (HCl salt) in 1 mL 1 N NaOH (for 2-fluorethylamine.HCl). The mixture was stirred overnight at room temperature and concentrated. The residue was dissolved in 2 mL DMSO, filtered and purified by HPLC (NH₃ buffers).

### Preparation of 7,9-Dibenzyl-6-(3,6-dihydro-2H-pyran-4-yl)-2-(3-hydroxyphenyl)-7,9-dihydro-8H-purin-8-one

### Example 66

To a stirred suspension of 6-(3,6-dihydro-2H-pyran-4-yl)-2-(3-hydroxyphenyl)-7,9-dihydro-8H-purin-8-one (100 mg, 0.3 mmol) and K₂CO₃ (41 mg, 0.2 mmol) in DMF (1 mL) was added benzyl bromide (33 mg, 0.3 mmol) and the mixture was stirred for 24 hrs. The reaction mixture was filtered and purified by preparative HPLC to give the 7,9-dibenzyl-6-(3,6-dihydro-2H-pyran-4-yl)-2-(3-hydroxyphenyl)-7,9-dihydro-8H-purin-8-one (5 mg, 12% yield), MS (ESI) m/z 494.3.

The compounds shown in Tables 1-5 below, were prepared according to the above procedures:

TABLE 1
1H-Pyrazolo[3,4-d]pyrimidine compounds

| Compound | Name | m/z | LC* (min) |
|---|---|---|---|
| 1 | 3-[4-(3,6-dihydro-2H-pyran-4-yl)-1-phenyl-1H- | 371.3 | 13.8 |
| | pyrazolo[3,4-d]pyrimidin-6-yl]phenol | | |
| 2 | 3-[1-phenyl-4-(tetrahydro-2H-pyran-4-yl)-1H- | 372.2 | 17.0 |
| | pyrazolo[3,4-d]pyrimidin-6-yl]phenol | | |
| 3 | N-{4-[1-(1-benzylpiperidin-4-yl)-4-(3,6-dihydro-2H- | 509.2 | 8.8 |
| | pyran-4-yl)-1H-pyrazolo[3,4-d]pyrimidin-6- | | |
| | yl]phenyl}acetamide | | |
| 4 | 1-(4-{4-(3,6-dihydro-2H-pyran-4-yl)-1-[1-(pyridin-3- | 525.1 | — |
| | ylmethyl)piperidin-4-yl]-1H-pyrazolo[3,4-d]pyrimidin- | | |
| | 6-yl}phenyl)-3-methylurea | | |
| 5 | 1-(4-{4-(3,6-dihydro-2H-pyran-4-yl)-1-[1-(pyridin-3- | 539.4 | — |
| | ylcarbonyl)piperidin-4-yl]-1H-pyrazolo[3,4- | | |
| | d]pyrimidin-6-yl}phenyl)-3-methylurea | | |
| 6 | 1-{4-[4-(3,6-dihydro-2H-pyran-4-yl)-1-(2,2,2- | 433.2 | — |
| | trifluoroethyl)-1H-pyrazolo[3,4-d]pyrimidin-6- | | |
| | yl]phenyl}-3-methylurea | | |
| 7 | 1-{4-[4-(3,6-dihydro-2H-pyran-4-yl)-1-(2,2,2- | 465.2 | — |
| | trifluoroethyl)-1H-pyrazolo[3,4-d]pyrimidin-6- | | |
| | yl]phenyl}-3-(2-fluoroethyl)urea | | |
| 8 | 1-{4-[4-(3,6-dihydro-2H-pyran-4-yl)-1-(2,2,2- | 496.2 | — |
| | trifluoroethyl)-1H-pyrazolo[3,4-d]pyrimidin-6- | | |
| | yl]phenyl}-3-pyridin-3-ylurea | | |
| 8a | 1-(pyridin-3-yl)-3-(4-(4-(tetrahydro-2H-pyran-4-yl)-1- | | |
| | (2,2,2-trifluoroethyl)-1H-pyrazolo[3,4-d]pyrimidin-6- | | |
| | yl)phenyl)urea, | | |
| 9 | 1-{4-[4-(3,6-dihydro-2H-pyran-4-yl)-1-(2,2,2- | 495.2 | — |
| | trifluoroethyl)-1H-pyrazolo[3,4-d]pyrimidin-6- | | |
| | yl]phenyl}-3-phenylurea | | |
| 9a | 1-{4-[4-(3,6-dihydro-2H-pyran-4-yl)-1-(2,2,2- | | |
| | trifluoroethyl)-1H-pyrazolo[3,4-d]pyrimidin-6- | | |
| | yl]phenyl}-3-(4-(4-methylpiperazin-1-yl)phenyl)urea | | |
| 9b | 1-{4-[4-(3,6-dihydro-2H-pyran-4-yl)-1-(2,2,2- | | |
| | trifluoroethyl)-1H-pyrazolo[3,4-d]pyrimidin-6- | | |
| | yl]phenyl}-3-(4-(4-methylpiperazine-1- | | |
| | carbonyl)phenyl)urea | | |
| 10 | 1-{4-[4-(3,6-dihydro-2H-pyran-4-yl)-1-(2,2,2- | 447.2 | — |
| | trifluoroethyl)-1H-pyrazolo[3,4-d]pyrimidin-6- | | |
| | yl]phenyl}-3-ethylurea | | |
| 10a | 2-hydroxyethyl {4-[4-(3,6-dihydro-2H-pyran-4-yl)-1- | | |
| | (2,2,2-trifluoroethyl)-1H-pyrazolo[3,4-d]pyrimidin-6- | | |
| | yl]phenyl}carbamate | | |
| 10b | 2-hydroxyethyl {4-[4-(tetrahydro-2H-pyran-4-yl)-1- | | |
| | (2,2,2-trifluoroethyl)-1H-pyrazolo[3,4-d]pyrimidin-6- | | |
| | yl]phenyl}carbamate | | |

*PRODIGY ODS3, 0.46 × 15 CM COLUMN 1.0 ML/MIN, 20 MIN GRADIENT ACN IN H₂O/TFA

TABLE 2
9H-Purine compounds

| Compound | Name | m/z | LC* (min) |
|---|---|---|---|
| 11 | 3-[6-(3,6-dihydro-2H-pyran-4-yl)-9H-purin-2-yl]phenol | 295.2 | 9.1 |
| 12 | 3-[9-(1-benzylpiperidin-4-yl)-6-(3,6-dihydro-2H- | 468.2 | — |
| | pyran-4-yl)-9H-purin-2-yl]phenol | | |
| 13 | 3-{6-(3,6-dihydro-2H-pyran-4-yl)-9-[1-(2- | 458.2 | — |
| | furylmethyl)piperidin-4-yl]-9H-purin-2-yl}phenol | | |
| 14 | 3-[6-(3,6-dihydro-2H-pyran-4-yl)-9-{1-[(6- | 554.3 | — |
| | morpholin-4-ylpyridin-3-yl)methyl]piperidin-4-yl}- | | |
| | 9H-purin-2-yl]phenol | | |
| 15 | 3-{6-(3,6-dihydro-2H-pyran-4-yl)-9-[1-(1H-pyrrol- | 457.3 | — |
| | 2-ylmethyl)piperidin-4-yl]-9H-purin-2-yl}phenol | | |
| 16 | 3-[6-(3,6-dihydro-2H-pyran-4-yl)-9-(1- | 392.2 | — |
| | methylpiperidin-4-yl)-9H-purin-2-yl]phenol | | |
| 17 | 3-{6-(3,6-dihydro-2H-pyran-4-yl)-9-[1-(1H- | 458.2 | — |
| | imidazol-5-ylmethyl)piperidin-4-yl]-9H-purin-2- | | |
| | yl}phenol | | |
| 18 | 3-{6-(3,6-dihydro-2H-pyran-4-yl)-9-[1-(4- | 482.3 | — |
| | methylbenzyl)piperidin-4-yl]-9H-purin-2-yl}phenol | | |
| 19 | 3-[9-{1-[(6-bromopyridin-3-yl)methyl]piperidin-4- | 549.1 | — |
| | yl}-6-(3,6-dihydro-2H-pyran-4-yl)-9H-purin-2- | | |
| | yl]phenol | | |
| 20 | 3-{9-[1-(3,4-difluorobenzyl)piperidin-4-yl]-6-(3,6- | 504.2 | — |
| | dihydro-2H-pyran-4-yl)-9H-purin-2-yl}phenol | | |
| 21 | {3-[9-(1-benzylpiperidin-4-yl)-6-(3,6-dihydro-2H- | 482.3 | — |
| | pyran-4-yl)-9H-purin-2-yl]phenyl}methanol | | |
| 22 | {3-[6-(3,6-dihydro-2H-pyran-4-yl)-9-{1-[(6- | 501.2 | — |
| | fluoropyridin-3-yl)methyl]piperidin-4-yl}-9H- | | |
| | purin-2-yl]phenyl}methanol | | |
| 23 | (3-{6-(3,6-dihydro-2H-pyran-4-yl)-9-[1-(pyridin-3- | 483.2 | — |
| | ylmethyl)piperidin-4-yl]-9H-purin-2- | | |
| | yl}phenyl)methanol | | |
| 24 | (3-{6-(3,6-dihydro-2H-pyran-4-yl)-9-[1-(pyridin-2- | 483.2 | — |
| | ylmethyl)piperidin-4-yl]-9H-purin-2- | | |
| | yl}phenyl)methanol | | |
| 25 | {3-[9-{1-[(6-bromopyridin-3-yl)methyl]piperidin- | 561.2 | — |
| | 4-yl}-6-(3,6-dihydro-2H-pyran-4-yl)-9H-purin-2- | | |
| | yl]phenyl}methanol | | |
| 26 | 1-{4-[6-(3,6-dihydro-2H-pyran-4-yl)-9-piperidin-4- | 503.3 | — |
| | yl-9H-purin-2-yl]phenyl}-3-piperidin-4-ylurea | | |
| 27 | benzyl 4-{[(4-{6-(3,6-dihydro-2H-pyran-4-yl)-9-[1- | 716.4 | — |
| | (1H-pyrrol-2-ylmethyl)piperidin-4-yl]-9H-purin-2- | | |
| | yl}phenyl)carbamoyl]amino}piperidine-1- | | |
| | carboxylate | | |
| 28 | 5-[9-(1-benzylpiperidin-4-yl)-6-(3,6-dihydro-2H- | 469.2 | — |
| | pyran-4-yl)-9H-purin-2-yl]pyridin-3-ol | | |
| 29 | 5-{6-(3,6-dihydro-2H-pyran-4-yl)-9-[1-(pyridin-3- | 470.2 | — |
| | ylmethyl)piperidin-4-yl]-9H-purin-2-yl}pyridin-3- | | |
| | ol | | |
| 30 | 5-[9-{1-[(6-bromopyridin-3-yl)methyl]piperidin-4- | 548.1 | — |
| | yl}-6-(3,6-dihydro-2H-pyran-4-yl)-9H-purin-2- | | |
| | yl]pyridin-3-ol | | |
| 31 | 5-{6-(3,6-dihydro-2H-pyran-4-yl)-9-[1-(pyridin-2- | 469.2 | — |
| | ylmethyl)piperidin-4-yl]-9H-purin-2-yl}pyridin-3- | | |
| | ol | | |
| 32 | 5-[6-(3,6-dihydro-2H-pyran-4-yl)-9-{1-[(6- | 500.2 | — |
| | methoxypyridin-3-yl)methyl]piperidin-4-yl}-9H- | | |
| | purin-2-yl]pyridin-3-ol | | |
| 33 | 5-[6-(3,6-dihydro-2H-pyran-4-yl)-9-{1-[(5-fluoro- | 526.3 | — |
| | 1H-indol-3-yl)methyl]piperidin-4-yl}-9H-purin-2- | | |
| | yl]pyridin-3-ol | | |
| 34 | 5-[9-{1-[(2-aminopyridin-3-yl)methyl]piperidin-4- | — | — |
| | yl}-6-(3,6-dihydro-2H-pyran-4-yl)-9H-purin-2- | | |
| | yl]pyridin-3-ol | | |
| 35 | 5-[9-{1-[(5-bromopyridin-3-yl)methyl]piperidin-4- | 485.2 | — |
| | yl}-6-(3,6-dihydro-2H-pyran-4-yl)-9H-purin-2- | | |
| | yl]pyridin-3-ol | | |
| 36 | 5-[6-(3,6-dihydro-2H-pyran-4-yl)-9-{1-[(2- | 500.2 | — |
| | methoxypyridin-3-yl)methyl]piperidin-4-yl}-9H- | | |
| | purin-2-yl]pyridin-3-ol | | |
| 37 | 5-[9-{1-[(6-chloropyridin-3-yl)methyl]piperidin-4- | 504.2 | — |
| | yl}-6-(3,6-dihydro-2H-pyran-4-yl)-9H-purin-2- | | |
| | yl]pyridin-3-ol | | |
| 40 | 3-[6-(3,6-dihydro-2H-pyran-4-yl)-9-piperidin-4-yl- | 378.2 | 6.8 |
| | 9H-purin-2-yl]phenol | | |
| 41 | 1-(4-{6-(3,6-dihydro-2H-pyran-4-yl)-9-[1-(1H- | 513.2 | 7.4 |
| | pyrrol-2-ylmethyl)piperidin-4-yl]-9H-purin-2- | | |
| | yl}phenyl)-3-methylurea | | |
| 42 | tert-butyl 4-{6-(3,6-dihydro-2H-pyran-4-yl)-2-[3- | 492.3 | 15.4 |
| | (hydroxymethyl)phenyl]-9H-purin-9-yl}piperidine- | | |
| | 1-carboxylate | | |
| 43 | {3-[6-(3,6-dihydro-2H-pyran-4-yl)-9-piperidin-4- | 392.3 | 6.7 |
| | yl-9H-purin-2-yl]phenyl}methanol | | |
| 44 | 5-[6-(3,6-dihydro-2H-pyran-4-yl)-9-piperidin-4-yl- | 379.3 | 4.4 |
| | 9H-purin-2-yl]pyridin-3-ol | | |
| 45 | {3-[6-(3,6-dihydro-2H-pyran-4-yl)-9-(2-piperidin- | 420.3 | 6.9 |
| | 1-ylethyl)-9H-purin-2-yl]phenyl}methanol | | |
| 46 | 3-[6-(3,6-dihydro-2H-pyran-4-yl)-9-(2-piperidin-1- | 406.4 | 7.2 |
| | ylethyl)-9H-purin-2-yl]phenol | | |

*PRODIGY ODS3, 0.46 × 15 CM COLUMN 1.0 ML/MIN, 20 MIN GRADIENT ACN IN H₂O/TFA

TABLE 3
3H-[1,2,3]Triazolo[4,5-d]pyrimidine compounds

| Com- | | | LC* |
|---|---|---|---|
| pound | Name | m/z | (min) |
| 47 | 3-[3-(1-benzylpiperidin-4-yl)-7-(3,6-dihydro-2H- | 469.2 | |
| | pyran-4-yl)-3H-[1,2,3]triazolo[4,5-d]pyrimidin-5- | | |
| | yl]phenol | | |
| 48 | 3-[7-(3,6-dihydro-2H-pyran-4-yl)-3H- | 296.1 | 11.0 |
| | [1,2,3]triazolo[4,5-d]pyrimidin-5-yl]phenol | | |
| 49 | {3-[7-(3,6-dihydro-2H-pyran-4-yl)-3H- | 310.1 | 10.4 |
| | [1,2,3]triazolo[4,5-d]pyrimidin-5- | | |
| | yl]phenyl}methanol | | |
| 50 | 5-[7-(3,6-dihydro-2H-pyran-4-yl)-3H- | 297.1 | 6.1 |
| | [1,2,3]triazolo[4,5-d]pyrimidin-5-yl]pyridin-3-ol | | |

*PRODIGY ODS3, 0.46 × 15 CM COLUMN 1.0 ML/MIN, 20 MIN GRADIENT ACN IN H₂O/TFA

TABLE 4
Thieno[3,2-d]pyrimidine compounds

| Com- | | | LC* |
|---|---|---|---|
| pound | Name | m/z | (min) |
| 51 | 1-(4-(4-(3,6-dihydro-2H-pyran-4-yl)thieno[3,2- | 381.1 | 2.13 |
| | d]pyrimidin-2-yl)phenyl)-3-ethylurea | | |
| 52 | 1-(4-(4-(3,6-dihydro-2H-pyran-4-yl)thieno[3,2- | 399.1 | 2.1 |
| | d]pyrimidin-2-yl)phenyl)-3-(2-fluoroethyl)urea | | |
| 53 | 1-(4-(4-(3,6-dihydro-2H-pyran-4-yl)thieno[3,2- | 429.1 | 2.35 |
| | d]pyrimidin-2-yl)phenyl)-3-phenylurea | | |
| 54 | 1-(4-(4-(3,6-dihydro-2H-pyran-4-yl)thieno[3,2- | 430.1 | 1.93 |
| | d]pyrimidin-2-yl)phenyl)-3-(pyridin-3-yl)urea | | |
| 55 | 1-(4-(4-(3,6-dihydro-2H-pyran-4-yl)thieno[3,2- | 430.1 | 1.89 |
| | d]pyrimidin-2-yl)phenyl)-3-(pyridin-4-yl)urea | | |
| 56 | 4-(4-(3,6-dihydro-2H-pyran-4-yl)thieno[3,2- | 310.1 | 1.99 |
| | d]pyrimidin-2-yl)aniline | | |
| 57 | N-(4-(4-(3,6-dihydro-2H-pyran-4-yl)thieno[3,2- | 352.1 | 2.12 |
| | d]pyrimidin-2-yl)phenyl)acetamide | | |
| 58 | methyl 4-(4-(3,6-dihydro-2H-pyran-4- | 368.1 | 2.24 |
| | yl)thieno[3,2-d]pyrimidin-2- | | |
| | yl)phenylcarbamate | | |
| 59 | 3-(4-(3,6-dihydro-2H-pyran-4-yl)thieno[3,2- | 311.1 | 2.08 |
| | d]pyrimidin-2-yl)phenol | | |
| 60 | 4-(4-(3,6-dihydro-2H-pyran-4-yl)thieno[3,2- | 311.1 | 2.07 |
| | d]pyrimidin-2-yl)phenol | | |
| 61 | 4-(3,6-dihydro-2H-pyran-4-yl)-2-(1H-indol-5- | 334.1 | 2.26 |
| | yl)thieno[3,2-d]pyrimidine | | |
| 62 | 5-(4-(3,6-dihydro-2H-pyran-4-yl)thieno[3,2- | 311.1 | 1.62 |
| | d]pyrimidin-2-yl)pyridin-2-amine | | |
| 63 | 2-hydoxyethyl-4-(4-(3,6-dihydro-2H-pyran-4- | | |
| | yl)thieno[3,2-d]pyrimidin-2-yl)phenylcarbamate | | |
| 64 | 1-(4-(4-(3,6-dihydro-2H-pyran-4-yl)thieno[3,2- | | |
| | d]pyrimidin-2-yl)phenyl)-3-(4-(4- | | |
| | methylpiperazin-1-yl)phenyl)urea | | |
| 65 | 1-(pyridin-3-yl)-3-(4-(4-(tetrahydro-2H-pyran-4- | | |
| | yl)thieno[3,2-d]pyrimidin-2-yl)phenyl)urea | | |

*PRODIGY ODS3, 0.46 × 15 CM COLUMN 1.0 ML/MIN, 20 MIN GRADIENT ACN IN H₂O/TFA

TABLE 5
8H-Purin-8-one compounds

| Com- | | | LC* |
|---|---|---|---|
| pound | Name | m/z | (min) |
| 38 | 6-(3,6-dihydro-2H-pyran-4-yl)-2-(3- | 309.1 | 8.6 |
| | hydroxyphenyl)-7,9-dihydro-8H-purin-8-one | | |
| 39 | 6-(3,6-dihydro-2H-pyran-4-yl)-2-[3- | 323.1 | 8.1 |
| | (hydroxymethyl)phenyl]-7,9-dihydro-8H- | | |
| | purin-8-one | | |
| 66 | 7,9-dibenzyl-6-(3,6-dihydro-2H-pyran-4-yl)-2-(3- | 462.2 | 2.23 |
| | hydroxyphenyl)-7,9-dihydro-8H-purin-8-one | | |
| 67 | 6-(3,6-dihydro-2H-pyran-4-yl)-2-(3- | 309.1 | 8.6 |
| | hydroxyphenyl)-7,9-dihydro-8H-purin-8-one | | |

*PRODIGY ODS3, 0.46 × 15 CM COLUMN 1.0 ML/MIN, 20 MIN GRADIENT ACN IN H₂O/TFA

Biological Evaluation—

mTOR Kinase Assay Methods

Human mTOR assays (See Toral-Barza, et al. Biochem Biophys. Res. Commun. 2005 Jun. 24; 332(1):304-10) with purified enzyme are performed in 96-well plates by DELFIA format as follows. Enzymes are first diluted in kinase assay buffer (10 mM HEPES (pH 7.4), 50 mM NaCl, 50 mM β-glycerophosphate, 10 mM MnCl₂, 0.5 mM DTT, 0.25 mM microcystin LR, and 100 mg/mL BSA). To each well, 12 μL of the diluted enzyme is mixed briefly with 0.5 μL test inhibitor or control vehicle dimethylsulfoxide (DMSO). The kinase reaction is initiated by adding 12.5 μL kinase assay buffer containing ATP and His6-S6K to give a final reaction volume of 25 μL containing 800 ng/mL FLAG-TOR, 100 mM ATP and 1.25 mM His6-S6K. The reaction plate is incubated for 2 hours (linear at 1-6 hours) at room temperature with gentle shaking and then terminated by adding 25 μL Stop buffer (20 mM HEPES (pH 7.4), 20 mM EDTA, 20 mM EGTA). The DELFIA detection of the phosphorylated (Thr-389) His6-S6K is performed at room temperature using a monoclonal anti-P(T389)-p70S6K antibody (1A5, Cell Signaling) labeled with Europium-N1-ITC (Eu) (10.4 Eu per antibody, PerkinElmer). The DELFIA Assay buffer and Enhancement solution can be purchased from PerkinElmer. 45 μL of the terminated kinase reaction mixture is transferred to a MaxiSorp plate (Nunc) containing 55 μL PBS. The His6-S6K is allowed to attach for 2 hours after which the wells are aspirated and washed once with PBS. 100 μL of DELFIA Assay buffer with 40 ng/mL Eu-P(T389)-S6K antibody is added. The antibody binding is continued for 1 hour with gentle agitation. The wells are then aspirated and washed 4 times with PBS containing 0.05% Tween-20 (PBST). 100 μL of DELFIA Enhancement solution is added to each well and the plates are read in a PerkinElmer Victor model plate reader. Data obtained is used to calculate enzymatic activity and enzyme inhibition by potential inhibitors.

### Fluorescence Polarization Assay for PI3K

This assay is used to determine the IC₅₀ of compounds of the present invention as it identifies inhibitors of PI3 kinase by measuring inhibition.

### Materials

Reaction Buffer: 20 mM HEPES, pH 7.5, 2 mM MgCl₂, 0.05% CHAPS; and 0.01% BME (added fresh) Stop/Detection Buffer: 100 mM HEPES, pH 7.5, 4 mM EDTA, 0.05% CHAPS; ATP 20 mM in water; PIP2 (diC8, cat# P-4508) 1 mM in water (MW=856.5); GST-GRP 1.75 mg/mL or 1.4 mg/mL in 10% glycerol; Red detector (TAMRA) 2.5 μM; Plate: Nunc 384 well black polypropylene fluorescence plate.

### Methods

The assay is run by placing 5 μL of diluted enzyme per well, then 5 μL of diluted compound (or 9.5 μL enzyme then 0.5 μL compound in DMSO) is added and mixed. Then, 10 μL substrate is added to start the reaction. The samples are incubated 30-60 minutes, then the reaction is stopped by adding 20 μL stop/detector mix. PI3K is diluted with reaction buffer (e.g., 5 μL or 7.5 μL PI3K into 620 μL reaction buffer), and 5 μL of diluted enzyme is used per well. 5 μL reaction buffer or drug diluted in buffer (e.g., 4 μL/100 so final DMSO is 1% in reaction) is added to each. Pipetting up and down mixes the samples. Alternatively, the enzyme can be diluted to 1215 μL. In this case 9.8 μL is added per well and 0.2 μL compound is added in DMSO. To prepare 1 mL of substrate solution, 955 μL reaction buffer, 40 μL PIP2, and 2.5 μL ATP are mixed. 10 μL of substrate is added to each well to start the reaction. This results in 20 μM PIP2, and 25 μM ATP per reaction.

Stop/detector mix is prepared by mixing 4 μL Red detector and 1.6 μL or 2.0 μL GST-GRP with 1 mL Stop buffer, which results in 10 nM probe and 70 nM GST-GRP). 20 μL of the stop/detector mix is added to each well to stop the reaction. The plates are read after 30-90 minutes keeping the red probe solutions dark. For the zero time point, stop/detector mix is added to the enzyme just before adding substrate. For an extra control, stop/detector mix is added to buffer (no enzyme) and substrate or to just buffer (no substrate). Pooled PI3K preparations had a protein concentration of 0.25 mg/mL. The recommended reaction has 0.06 μL per 20 μL (0.015 μg/20 μL) or 0.01125 μg/15 μL or 0.75 μg/mL. Plates are read on machines with filters for Tamra. The units are mP with no enzyme controls reading app 190-220 mP units. Fully active enzyme reduces fluorescence polarization down to 70-100 mP after 30 minutes. An active compound raises the mP values halfway to control or to 120-150 mP units.

### In Vitro Cell Culture Growth Assay Methods:

Human tumor cell lines used include prostate lines LNCap and PC3MM2, breast lines MDA468, MCF7, renal line HTB44 (A498), colon line HCT116, and ovarian line OVCAR3. Cells were plated in 96-well culture plates. One day following plating, the inhibitors were added to cells. Three days after drug treatment, viable cell densities were determined by metabolic conversion (by viable cells) of the dye MTS, a well-established cell proliferation assay. The assays were performed using an assay kit purchased from Promega Corp. (Madison, Wis.) following the protocol supplied with the kit. The MTS assay results were read in a 96-well plate reader by measuring absorbance at 490 nm. The effect of each treatment was calculated as percent of control growth relative to the vehicle-treated cells grown in the same culture plate. The drug concentration that conferred 50% inhibition of growth was determined as IC₅₀ (μg/ml).

Table 6 shows the results of the described biological assays.

TABLE 6

| | TOR | PI3 | PI3 | | |
|---|---|---|---|---|---|
| Com- | Kinase | Kinase α | Kinase γ | MDA468 | LNCap |
| pound | IC₅₀ (μM) | IC₅₀ (nM) | IC₅₀ (nM) | IC₅₀ (μM) | IC₅₀ (μM) |
| 1 | 0.017 | 48 | 1894 | 8.25 | 2.8 |
| 2 | 0.67 | 855 | >10000 | 50 | 11 |
| 3 | 0.23 | 3089 | 3020 | 11 | 5 |
| 4 | 0.017 | 2824 | 3225 | 4.2 | 0.9 |
| 5 | 0.0084 | 2099 | 1179 | 4 | 2.8 |
| 6 | 0.0024 | 212 | 3026 | 0.95 | 0.26 |
| 7 | 0.0014 | 2333 | 1013 | 0.22 | 0.072 |
| 8 | 0.00075 | 66 | >10000 | 7 | 0.38 |
| 9 | 0.0026 | 43 | 74 | 0.23 | 0.096 |
| 10 | 0.0024 | 851 | >10000 | 0.68 | 0.21 |
| 10a | 3.550 | 696 | 3358 | 1200 | 170 |
| 11 | 1.4 | 85 | 1247 | | |
| 12 | 1.8 | 1766 | 5000 | | |
| 13 | 1.4 | 832 | 859 | | |
| 14 | 0.86 | 1493 | >10000 | | |
| 15 | 1.4 | 280 | 736 | | |
| 16 | 2.3 | 1632 | 3200 | | |
| 17 | 1.2 | 1063 | 2580 | | |
| 18 | 2.1 | 12000 | 12000 | | |
| 19 | 0.69 | 5000 | >10000 | | |
| 20 | 4 | 7003 | >10000 | | |
| 21 | 3.5 | 952 | 4362 | | |
| 22 | 1.9 | 1052 | 5405 | | |
| 23 | 3.8 | 1488 | 7370 | | |
| 24 | 1.7 | 1689 | 7616 | | |
| 25 | 0.89 | 228 | 1336 | | |
| 26 | >4.00000 | 2670 | 7000 | | |
| 27 | >4.00000 | 3026 | 1076 | | |
| 28 | 3.6 | 384 | 2395 | | |
| 29 | 2.4 | 525 | 2490 | | |
| 30 | 2.0 | 275 | 2470 | | |
| 31 | 1.6 | 619 | 2988 | | |
| 32 | 1.7 | 858 | 2532 | | |
| 33 | 12 | 265 | 385 | | |
| 34 | 13 | 526 | 1500 | | |
| 35 | 9.8 | 1337 | 6741 | | |
| 36 | 2.8 | 506 | 2485 | | |
| 37 | 1.6 | 437 | 719 | | |
| 38 | 0.19 | 152 | 543 | | |
| 39 | 1.3 | 524 | 819 | | |
| 40 | 1.4 | 540 | 3156 | | |
| 41 | 0.13 | 543 | 3112 | | |
| 43 | >4.00000 | 574 | 2064 | | |
| 44 | 4.8 | 628 | 2630 | | |
| 45 | >4.00000 | 1352 | 9000 | | |
| 46 | >4.00000 | 1348 | 15000 | | |
| 47 | 0.23 | 998 | 1500 | | |
| 48 | 0.032 | 93 | 257 | | |
| 49 | 0.34 | 390 | 1036 | | |
| 50 | 0.36 | 78 | 100 | | |
| 66 | 14 | 2425 | 1611 | | |
| 67 | 0.28 | 169 | 414 | >10 uM | |

While particular aspects of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

Throughout this application, various publications are referenced. The disclosures of these publications in their entireties are hereby incorporated by reference into this application in order to more fully describe the state of the art as known to those skilled therein as of the date of the invention described and claimed herein.

What is claimed is:

1. A compound of the Formula 1: 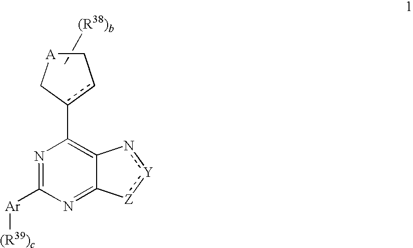
or a pharmaceutically acceptable salt thereof, wherein
A is —O—, —CH₂—O—, —CH₂—CH₂—O—, —CH₂—O—CH₂—, or —CH₂—S—;
the dashed lines - - - - - independently represents an optional second bond;
R³⁸ is independently C₁-C₆alkyl; C₂-C₆alkenyl; C₂-C₆alkynyl; or C₃-C₈cycloalkyl any of which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)C₁-C₆alkyl, C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂;
b is 0, 1, or 2;
Ar is phenyl, naphthyl, or nitrogen-containing mono- or bicyclic heteroaryl;
R³⁹ is independently halogen; one of the meanings of R³⁸; C₁-C₆alkoxy which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₁-C₆alkoxycarbonyl; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; (C₆-C₁₄)aryloxy which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; hydroxyl; NR⁴⁰R⁴¹; NO₂; CN; —C(O)NR⁴⁰R⁴¹; R⁴²C(O)NH—; CO₂H; CF₃; CF₃O; (C₁-C₆alkyl)thio; —SO₂NR⁴⁰R⁴¹; —NHC(O)NR⁴⁰R⁴¹; —NHC(O)OR⁴²; —NH(SO₂)NH—(C₁-C₆alkyl); —NH(SO₂)NH—(C₆-C₁₄aryl); —NHC(S)—NH—(C₁-C₆alkyl); —N═C(S—C₁-C₆alkyl)NH—(C₁-C₆alkyl); —S(O)_{d}—(C₆-C₁₄aryl); —S(O)_{d}—(C₁-C₉heteroaryl); or —N(H)—C(═N—(CN))—O—(C₆-C₁₄aryl);
c is 0, 1, 2, 3, 4, or 5;
each d is independently 1 or 2;
R⁴⁰ and R⁴¹ are each independently H; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, —NO₂, R⁴⁶ or C(O)R⁴⁷; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₃-C₈cycloalkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; 4- to 7-membered monocyclic heterocycle group which is unsubstituted or is substituted with from 1 to 3 substituents selected from C₁-C₈acyl, C₁-C₆alkyl, heterocyclylalkyl, (C₆-C₁₄aryl)alkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂; or R⁴⁰ and R⁴¹ when taken together with the nitrogen to which they are attached can form a 3- to 7-membered nitrogen containing heterocycle wherein up to two of the carbon atoms of the heterocycle can be replaced with —N(R⁴³)—, —O—, or —S(O)_{d}—;
R⁴² is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; or C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂;
R⁴³ is hydrogen; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₃-C₈cycloalkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; amino(C₁-C₆alkyl)-; or arylamino;
X, Y, and Z are independently N(R⁴⁴)—; C(R⁴⁵); C═O and S;
R⁴⁴ is hydrogen; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; heterocyclylalkyl; 4- to 7-membered monocyclic heterocycle group which is unsubstituted or is substituted with from 1 to 3 substituents independently selected from C₁-C₈acyl, C₁-C₆alkyl, heterocyclylalkyl, wherein the ring portion of the heterocyclylalkyl group is unsubstituted or is substituted by 1 to 3 substituents independently selected from halogen, —NH₂, —O(C₁-C₆alkyl), C₁-C₆alkyl, 4- to 7-membered monocyclic heterocycle, and C₃-C₈cycloalkyl, (C₆-C₁₄aryl)alkyl, wherein the ring portion of the (C₆-C₁₄aryl)alkyl group is unsubstituted or is substituted by 1 to 3 substituents independently selected from halogen, —NH₂, —O(C₁-C₆alkyl), C₁-C₆alkyl, 4- to 7-membered monocyclic heterocycle, and C₃-C₈cycloalkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂; or C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₆-C₁₄)arylalkyl-O—C(O)—, (C₁-C₆alkyl)carboxyamido-, —C(O)NH₂, alkylcarboxamido-, or —NO₂;
R⁴⁵ is hydrogen; or is C₁-C₆alkyl; C₂-C₆alkenyl; or C₂-C₆alkynyl each of which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; and
R⁴⁶ is piperazinyl optionally substituted with 1 or 2 C₁-C₆alkyl; —O(C₂-C₃alkylene)N(C₁-C₆alkyl)(C₁-C₆alkyl); or —(C₁-C₃alkylene)N(C₁-C₆alkyl)(C₁-C₆alkyl); and
R⁴⁷ is piperazinyl optionally substituted with 1 or 2 C₁-C₆alkyl; or —N(C₁-C₃alkyl)-C₂-C₃alkylene-N(C₁-C₆alkyl)(C₁-C₆alkyl).

2. A compound of the Formula 2: 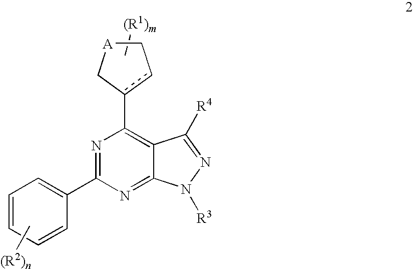
or a pharmaceutically acceptable salt thereof, wherein
A is —O—, —CH₂—O—, —CH₂—CH₂—O—, —CH₂—O—CH₂—, or —CH₂—S—;
the dashed line - - - - - represents an optional second carbon to carbon bond;
R¹ is independently C₁-C₆alkyl; C₂-C₆alkenyl; C₂-C₆alkynyl; or C₃-C₈cycloalkyl any of which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)C₁-C₆alkyl, C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂;
m is 0, 1, or 2;
R² is independently halogen; one of the meanings of R¹; C₁-C₆alkoxy which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₁-C₆alkoxycarbonyl; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; (C₆-C₁₄)aryloxy which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; hydroxyl; NR⁵R⁶; NO₂; CN; —C(O)NR⁵R⁶; R⁷C(O)NH—; CO₂H; CF₃; CF₃O; (C₁-C₆alkyl)thio; —SO₂NR⁵R⁶; —NHC(O)NR⁵R⁶; —NHC(O)OR⁷; —NH(SO₂)NH—(C₁-C₆alkyl); —NH(SO₂)NH—(C₆-C₁₄aryl); —NHC(S)—NH—(C₁-C₆alkyl); —N═C(S—C₁-C₆alkyl)NH—(C₁-C₆alkyl); —S(O)ₖ—(C₆-C₁₄aryl); S(O)ₖ—(C₁-C₉heteroaryl); or —N(H)—C(═N—(CN))—O—(C₆-C₁₄aryl);
n is 0, 1, 2, 3, 4, or 5;
each k is independently 1 or 2;
R⁵ and R⁶ are each independently H; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, —NO₂, R⁴⁶ or C(O)R⁴⁷; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₃-C₈cycloalkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; 4- to 7-membered monocyclic heterocycle group which is unsubstituted or is substituted with from 1 to 3 substituents selected from C₁-C₈acyl, C₁-C₆alkyl, heterocyclylalkyl, (C₆-C₁₄aryl)alkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂; or R⁵ and R⁶ when taken together with the nitrogen to which they are attached can form a 3- to 7-membered nitrogen containing heterocycle wherein up to two of the carbon atoms of the heterocycle can be replaced with —N(R⁸)—, —O—, or —S(O)ₖ—;
R⁷ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; or C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂;
R⁸ is hydrogen; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₃-C₈cycloalkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; amino(C₁-C₆alkyl)-; or arylamino;
R³ is hydrogen; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; heterocyclylalkyl; 4- to 7-membered monocyclic heterocycle group which is unsubstituted or is substituted with from 1 to 3 substituents independently selected from C₁-C₈acyl, C₁-C₆alkyl, heterocyclylalkyl, wherein the ring portion of the heterocyclylalkyl group is unsubstituted or is substituted by 1 to 3 substituents independently selected from halogen, —NH₂, —O(C₁-C₆alkyl), C₁-C₆alkyl, 4- to 7-membered monocyclic heterocycle, and C₃-C₈cycloalkyl, (C₆-C₁₄aryl)alkyl, wherein the ring portion of the (C₆-C₁₄aryl)alkyl group is unsubstituted or is substituted by 1 to 3 substituents independently selected from halogen, —NH₂, —O(C₁-C₆alkyl), C₁-C₆alkyl, 4- to 7-membered monocyclic heterocycle, and C₃-C₈cycloalkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂; or C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₆-C₁₄)arylalkyl-O—C(O)—, (C₁-C₆alkyl)carboxyamido-, —C(O)NH₂, alkylcarboxamido-, or —NO₂;
R⁴ is hydrogen; or is C₁-C₆alkyl; C₂-C₆alkenyl; or C₂-C₆alkynyl each of which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂;
R⁴⁶ is piperazinyl optionally substituted with 1 or 2 C₁-C₆alkyl; —O(C₂-C₃alkylene)N(C₁-C₆alkyl)(C₁-C₆alkyl); or —(C₁-C₃alkylene)N(C₁-C₆alkyl)(C₁-C₆alkyl); and
R⁴⁷ is piperazinyl optionally substituted with 1 or 2 C₁-C₆alkyl; or —N(C₁-C₃alkyl)-C₂-C₃alkylene-N(C₁-C₆alkyl)(C₁-C₆alkyl).

3. The compound of claim 2, wherein m is 0.

4. The compound of claim 2, wherein n is 1.

5. The compound of claim 2, wherein A is —CH₂—O—.

6. The compound of claim 2, wherein the dashed line - - - - - represents a second carbon to carbon bond.

7. The compound of claim 2, wherein, R² is —NHC(O)NR⁵R⁶.

8. The compound of claim 7, wherein R⁵ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂.

9. The compound of claim 8, wherein R⁵ is methyl.

10. The compound of claim 8, wherein R⁵ is 1-fluoroethyl.

11. The compound of claim 8, wherein R⁵ is phenyl.

12. The compound of claim 8, wherein R⁵ is 3-pyridyl.

13. The compound of claim 7, wherein R⁶ is H.

14. The compound of claim 2, wherein R³ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂.

15. The compound of claim 14, wherein R³ is 1,1,1,-trifluoroethyl.

16. The compound of claim 2, wherein R⁴ is H.

17. The compound of claim 2, wherein m is 0; n is 1; A is —CH₂—O—; the dashed line - - - - - represents a second carbon to carbon bond; R² is —NHC(O)NR⁵R⁶; R⁵ is selected from the group consisting of methyl, 1-fluoroethyl, phenyl, and 3-pyridyl; R⁶ is H; R³ is 1,1,1,-trifluoroethyl; and R⁴ is H.

18. A compound selected from the group consisting of:
3-[4-(3,6-dihydro-2H-pyran-4-yl)-1-phenyl-1H-pyrazolo[3,4-d]pyrimidin-6-yl]phenol;
3-[1-phenyl-4-(tetrahydro-2H-pyran-4-yl)-1H-pyrazolo[3,4-d]pyrimidin-6-yl]phenol;
N-{4-[1-(1-benzylpiperidin-4-yl)-4-(3,6-dihydro-2H-pyran-4-yl)-1H-pyrazolo[3,4-d]pyrimidin-6-yl]phenyl}acetamide;
1-(4-{4-(3,6-dihydro-2H-pyran-4-yl)-1-[1-(pyridin-3-ylmethyl)piperidin-4-yl]-1H-pyrazolo[3,4-d]pyrimidin-6-yl}phenyl)-3-methylurea;
1-(4-{4-(3,6-dihydro-2H-pyran-4-yl)-1-[1-(pyridin-3-ylcarbonyl)piperidin-4-yl]-1H-pyrazolo[3,4-d]pyrimidin-6-yl}phenyl)-3-methylurea;
1-{4-[4-(3,6-dihydro-2H-pyran-4-yl)-1-(2,2,2-trifluoroethyl)-1H-pyrazolo[3,4-d]pyrimidin-6-yl]phenyl}-3-methylurea;
1-{4-[4-(3,6-dihydro-2H-pyran-4-yl)-1-(2,2,2-trifluoroethyl)-1H-pyrazolo[3,4-d]pyrimidin-6-yl]phenyl}-3-(2-fluoroethyl)urea;
1-{4-[4-(3,6-dihydro-2H-pyran-4-yl)-1-(2,2,2-trifluoroethyl)-1H-pyrazolo[3,4-d]pyrimidin-6-yl]phenyl}-3-pyridin-3-ylurea;
1-{4-[4-(3,6-dihydro-2H-pyran-4-yl)-1-(2,2,2-trifluoroethyl)-1H-pyrazolo[3,4-d]pyrimidin-6-yl]phenyl}-3-phenylurea;
1-{4-[4-(3,6-dihydro-2H-pyran-4-yl)-1-(2,2,2-trifluoroethyl)-1H-pyrazolo[3,4-d]pyrimidin-6-yl]phenyl}-3-ethylurea;
1-{4-[4-(3,6-dihydro-2H-pyran-4-yl)-1-(2,2,2-trifluoroethyl)-1H-pyrazolo[3,4-d]pyrimidin-6-yl]phenyl}-3-(4-(4-methylpiperazin-1-yl)phenyl)urea;
1-{4-[4-(3,6-dihydro-2H-pyran-4-yl)-1-(2,2,2-trifluoroethyl)-1H-pyrazolo[3,4-d]pyrimidin-6-yl]phenyl}-3-(4-(4-methylpiperazine-1-carbonyl)phenyl)urea;
2-hydroxyethyl{4-[4-(3,6-dihydro-2H-pyran-4-yl)-1-(2,2,2-trifluoroethyl)-1H-pyrazolo[3,4-d]pyrimidin-6-yl]phenyl}carbamate;
2-hydroxyethyl{4-[4-(tetrahydro-2H-pyran-4-yl)-1-(2,2,2-trifluoroethyl)-1H-pyrazolo[3,4-d]pyrimidin-6-yl]phenyl}carbamate; and
1-(pyridin-3-yl)-3-(4-(4-(tetrahydro-2H-pyran-4-yl)-1-(2,2,2-trifluoroethyl)-1H-pyrazolo[3,4-d]pyrimidin-6-yl)phenyl)urea.

19. A compound of the Formula 3: 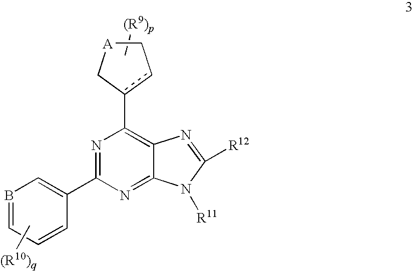
or a pharmaceutically acceptable salt thereof, wherein
A is —O—, —CH₂—O—, —CH₂—CH₂—O—, —CH₂—O—CH₂—, or —CH₂—S—;
the dashed line - - - - - represents an optional second carbon to carbon bond;
R⁹ is C₁-C₆alkyl; C₂-C₆alkenyl; C₂-C₆alkynyl; or C₃-C₈cycloalkyl each of which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)C₁-C₆alkyl, C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂;
p is 0, 1, or 2;
B is N or CH;
R¹⁰ is independently halogen; one of the meanings of R⁹; C₁-C₆alkoxy which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₁-C₆alkoxycarbonyl; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; (C₆-C₁₄)aryloxy which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; hydroxyl; NR¹³R¹⁴; NO₂; CN; —C(O)NR¹³R¹⁴; R¹⁵C(O)NH—; CO₂H; CF₃; CF₃O; (C₁-C₆alkyl)thio; —SO₂NR¹³R¹⁴; —NHC(O)NR¹³R¹⁴; —NHC(O)OR¹⁵; —NH(SO₂)NH—(C₁-C₆alkyl); —NH(SO₂)NH—(C₆-C₁₄aryl); —NHC(S)—NH—(C₁-C₆alkyl); —N═C(S—C₁-C₆alkyl)NH—(C₁-C₆alkyl); —S(O)ᵣ—(C₆-C₁₄aryl); S(O)ᵣ—(C₁-C₉heteroaryl); or —N(H)—C(═N—(CN))—O—(C₆-C₁₄aryl);
q is 0, 1, 2, 3, 4, or 5;
each r is independently 1 or 2;
R¹³ and R¹⁴ are each independently H; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, —NO₂, R⁴⁶ or C(O)R⁴⁷; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; 4- to 7-membered monocyclic heterocycle group which is unsubstituted or is substituted with from 1 to 3 substituents selected from C₁-C₈acyl, C₁-C₆alkyl, heterocyclylalkyl, (C₆-C₁₄aryl)alkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂; or R¹³ and R¹⁴ when taken together with the nitrogen to which they are attached can form a 3- to 7-membered nitrogen containing heterocycle wherein up to two of the carbon atoms of the heterocycle can be replaced with —N(R¹⁶)—, —O—, or —S(O)ᵣ—;
R¹⁵ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; or C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂;
R¹⁶ is hydrogen; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₃-C₈cycloalkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; amino(C₁-C₆alkyl)-; or arylamino;
R¹¹ is hydrogen; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; heterocyclylalkyl; 4- to 7-membered monocyclic heterocycle group which is unsubstituted or is substituted with from 1 to 3 substituents independently selected from C₁-C₈acyl, C₁-C₆alkyl, heterocyclylalkyl, wherein the ring portion of the heterocyclylalkyl group is unsubstituted or is substituted by 1 to 3 substituents independently selected from halogen, —NH₂, —O(C₁-C₆alkyl), C₁-C₆alkyl, 4- to 7-membered monocyclic heterocycle, and C₃-C₈cycloalkyl, (C₆-C₁₄aryl)alkyl, wherein the ring portion of the (C₆-C₁₄aryl)alkyl group is unsubstituted or is substituted by 1 to 3 substituents independently selected from halogen, —NH₂, —O(C₁-C₆alkyl), C₁-C₆alkyl, 4- to 7-membered monocyclic heterocycle, and C₃-C₈cycloalkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂; or C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₆-C₁₄)arylalkyl-O—C(O)—, (C₁-C₆alkyl)carboxyamido-, —C(O)NH₂, alkylcarboxamido-, or —NO₂;
R¹² is H or hydroxyl;
R⁴⁶ is piperazinyl optionally substituted with 1 or 2 C₁-C₆alkyl; —O(C₂-C₃alkylene)N(C₁-C₆alkyl)(C₁-C₆alkyl); or —(C₁-C₃alkylene)N(C₁-C₆alkyl)(C₁-C₆alkyl); and
R⁴⁷ is piperazinyl optionally substituted with 1 or 2 C₁-C₆alkyl; or —N(C₁-C₃alkyl)-C₂-C₃alkylene-N(C₁-C₆alkyl)(C₁-C₆alkyl).

20. The compound of claim 19, wherein p is 0.

21. The compound of claim 19, wherein, q is 1.

22. The compound of claim 19, wherein A is —CH₂—O—.

23. The compound of claim 19, wherein the dashed line - - - - - represents a second carbon-to-carbon bond.

24. The compound of claim 19, wherein R¹⁰ is —NHC(O)NR¹³R¹⁴.

25. The compound of claim 24, wherein R¹³ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂.

26. The compound of claim 25, wherein R¹³ is methyl.

27. The compound of claim 25, wherein R¹³ is 1-fluoroethyl.

28. The compound of claim 25, wherein R¹³ is phenyl.

29. The compound of claim 25, wherein R¹³ is 3-pyridyl.

30. The compound of claim 24, wherein R¹⁴ is H.

31. The compound of claim 19, wherein R¹¹ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂.

32. The compound of claim 19, wherein R¹¹ is 1,1,1,-trifluoroethyl.

33. The compound of claim 19, wherein R¹² is H.

34. The compound of claim 19, wherein p is 0; q is 1; A is —CH₂—O—; the dashed line - - - - - represents a second carbon to carbon bond; R¹⁰ is —NHC(O)NR¹³R¹⁴; R¹³ is selected from the group consisting of methyl, 1-fluoroethyl, phenyl, and 3-pyridyl; R¹⁴ is H; R¹¹ is 1,1,1,-trifluoroethyl; and R¹² is H.

35. A compound selected from the group consisting of:
3-[6-(3,6-dihydro-2H-pyran-4-yl)-9H-purin-2-yl]phenol;
3-[6-(3,6-dihydro-2H-pyran-4-yl)-9-piperidin-4-yl-9H-purin-2-yl]phenol;
3-[9-(1-benzylpiperidin-4-yl)-6-(3,6-dihydro-2H-pyran-4-yl)-9H-purin-2-yl]phenol;
3-{6-(3,6-dihydro-2H-pyran-4-yl)-9-[1-(2-furylmethyl)piperidin-4-yl]-9H-purin-2-yl}phenol;
3-[6-(3,6-dihydro-2H-pyran-4-yl)-9-{1-[(6-morpholin-4-ylpyridin-3-yl)methyl]piperidin-4-yl}-9H-purin-2-yl]phenol;
3-{6-(3,6-dihydro-2H-pyran-4-yl)-9-[1-(1H-pyrrol-2-ylmethyl)piperidin-4-yl]-9H-purin-2-yl}phenol;
3-[6-(3,6-dihydro-2H-pyran-4-yl)-9-(1-methylpiperidin-4-yl)-9H-purin-2-yl]phenol;
3-{6-(3,6-dihydro-2H-pyran-4-yl)-9-[1-(1H-imidazol-5-ylmethyl)piperidin-4-yl]-9H-purin-2-yl}phenol;
3-{6-(3,6-dihydro-2H-pyran-4-yl)-9-[1-(4-methylbenzyl)piperidin-4-yl]-9H-purin-2-yl}phenol;
3-[9-{1-[(6-bromopyridin-3-yl)methyl]piperidin-4-yl}-6-(3,6-dihydro-2H-pyran-4-yl)-9H-purin-2-yl]phenol;
3-{9-[1-(3,4-difluorobenzyl)piperidin-4-yl]-6-(3,6-dihydro-2H-pyran-4-yl)-9H-purin-2-yl}phenol;
{3-[9-(1-benzylpiperidin-4-yl)-6-(3,6-dihydro-2H-pyran-4-yl)-9H-purin-2-yl]phenyl}methanol;
{3-[6-(3,6-dihydro-2H-pyran-4-yl)-9-{1-[(6-fluoropyridin-3-yl)methyl]piperidin-4-yl}-9H-purin-2-yl]phenyl}methanol;
(3-{6-(3,6-dihydro-2H-pyran-4-yl)-9-[1-(pyridin-3-ylmethyl)piperidin-4-yl]-9H-purin-2-yl}phenyl)methanol;
(3-{6-(3,6-dihydro-2H-pyran-4-yl)-9-[1-(pyridin-2-ylmethyl)piperidin-4-yl]-9H-purin-2-yl}phenyl)methanol;
{3-[9-{1-[(6-bromopyridin-3-yl)methyl]piperidin-4-yl}-6-(3,6-dihydro-2H-pyran-4-yl)-9H-purin-2-yl]phenyl}methanol;
tert-butyl 4-{6-(3,6-dihydro-2H-pyran-4-yl)-2-[3-(hydroxymethyl)phenyl]-9H-purin-9-yl}piperidine-1-carboxylate;
{3-[6-(3,6-dihydro-2H-pyran-4-yl)-9-piperidin-4-yl-9H-purin-2-yl]phenyl}methanol;
1-(4-{6-(3,6-dihydro-2H-pyran-4-yl)-9-[1-(1H-pyrrol-2-ylmethyl)piperidin-4-yl]-9H-purin-2-yl}phenyl)-3-methylurea;
1-{4-[6-(3,6-dihydro-2H-pyran-4-yl)-9-piperidin-4-yl-9H-purin-2-yl]phenyl}-3-piperidin-4-ylurea;
benzyl 4-{[(4-{6-(3,6-dihydro-2H-pyran-4-yl)-9-[1-(1H-pyrrol-2-ylmethyl)piperidin-4-yl]-9H-purin-2-yl}phenyl)carbamoyl]amino}piperidine-1-carboxylate;
5-[6-(3,6-dihydro-2H-pyran-4-yl)-9-piperidin-4-yl-9H-purin-2-yl]pyridin-3-ol;
{3-[6-(3,6-dihydro-2H-pyran-4-yl)-9-(2-piperidin-1-ylethyl)-9H-purin-2-yl]phenyl}methanol;
5-[9-(1-benzylpiperidin-4-yl)-6-(3,6-dihydro-2H-pyran-4-yl)-9H-purin-2-yl]pyridin-3-ol;
5-{6-(3,6-dihydro-2H-pyran-4-yl)-9-[1-(pyridin-3-ylmethyl)piperidin-4-yl]-9H-purin-2-yl}pyridin-3-ol;
5-[9-{1-[(6-bromopyridin-3-yl)methyl]piperidin-4-yl}-6-(3,6-dihydro-2H-pyran-4-yl)-9H-purin-2-yl]pyridin-3-ol;
5-{6-(3,6-dihydro-2H-pyran-4-yl)-9-[1-(pyridin-2-ylmethyl)piperidin-4-yl]-9H-purin-2-yl}pyridin-3-ol;
3-[6-(3,6-dihydro-2H-pyran-4-yl)-9-(2-piperidin-1-ylethyl)-9H-purin-2-yl]phenol;
5-[6-(3,6-dihydro-2H-pyran-4-yl)-9-{1-[(6-methoxypyridin-3-yl)methyl]piperidin-4-yl}-9H-purin-2-yl]pyridin-3-ol;
5-[6-(3,6-dihydro-2H-pyran-4-yl)-9-{1-[(5-fluoro-1H-indol-3-yl)methyl]piperidin-4-yl}-9H-purin-2-yl]pyridin-3-ol;
5-[9-{1-[(2-aminopyridin-3-yl)methyl]piperidin-4-yl}-6-(3,6-dihydro-2H-pyran-4-yl)-9H-purin-2-yl]pyridin-3-ol;
5-[9-{1-[(5-bromopyridin-3-yl)methyl]piperidin-4-yl}-6-(3,6-dihydro-2H-pyran-4-yl)-9H-purin-2-yl]pyridin-3-ol;
5-[6-(3,6-dihydro-2H-pyran-4-yl)-9-{1-[(2-methoxypyridin-3-yl)methyl]piperidin-4-yl}-9H-purin-2-yl]pyridin-3-ol;
5-[9-{1-[(6-chloropyridin-3-yl)methyl]piperidin-4-yl}-6-(3,6-dihydro-2H-pyran-4-yl)-9H-purin-2-yl]pyridin-3-ol;
6-(3,6-dihydro-2H-pyran-4-yl)-2-(3-hydroxyphenyl)-7,9-dihydro-8H-purin-8-one;
6-(3,6-dihydro-2H-pyran-4-yl)-2-[3-(hydroxymethyl)phenyl]-7,9-dihydro-8H-purin-8-one;
1-(4-(6-(3,6-dihydro-2H-pyran-4-yl)-9-ethyl-9H-purin-2-yl)phenyl)-3-(4-(4-methylpiperazin-1-yl)phenyl)urea;
1-(4-(6-(3,6-dihydro-2H-pyran-4-yl)-9-ethyl-9H-purin-2-yl)phenyl)-3-(pyridin-4-yl)urea; and
1-(4-(6-(3,6-dihydro-2H-pyran-4-yl)-9-ethyl-9H-purin-2-yl)phenyl)-3-(4-(4-methylpiperazine-1-carbonyl)phenyl)urea.

36. A compound of the Formula 4: 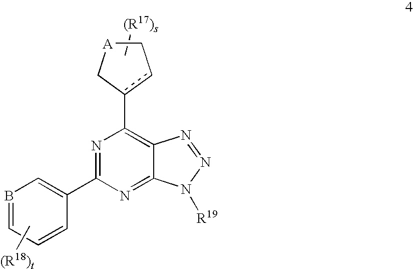
or a pharmaceutically acceptable salt thereof, wherein
A is —O—, —CH₂—O—, —CH₂—CH₂—O—, —CH₂—O—CH₂—, or —CH₂—S—;
the dashed line - - - - - represents an optional second carbon to carbon bond;
R¹⁷ is C₁-C₆alkyl; C₂-C₆alkenyl; C₂-C₆alkynyl; or C₃-C₈cycloalkyl each of which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)C₁-C₆alkyl, C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂;
s is 0, 1, or 2;
B is N or CH;
R¹⁸ is independently halogen; one of the meanings of R¹⁷; C₁-C₆alkoxy which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₁-C₆alkoxycarbonyl; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; (C₆-C₁₄)aryloxy which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, di(C₁-C₆alkyl)amino-, —COOH, —C(O)O—(C₁-C₆alkyl), —OC(O)(C₁-C₆alkyl), N—(C₁-C₆alkyl)amido-, —C(O)NH₂, alkylcarboxamido-, or —NO₂; hydroxyl; NR²⁰R²¹; NO₂; CN; —C(O)NR²⁰R²¹; R²²C(O)NH—; CO₂H; CF₃; CF₃O; C₁-C₆alkylthio; —SO₂NR²⁰R²¹; NHC(O)NR²⁰R²¹; —NHC(O)OR²²; —NH(SO₂)NH—(C₁-C₆alkyl); —NH(SO₂)NH—(C₆-C₁₄aryl); —NHC(S)—NH—(C₁-C₆alkyl); —N═C(S—C₁-C₆alkyl)NH—(C₁-C₆alkyl); —S(O)ᵤ—(C₆-C₁₄aryl); S(O)ᵤ—(C₁-C₉heteroaryl); or —N(H)—C(═N—(CN))—O—(C₆-C₁₄aryl);
t is 0, 1, 2, 3, 4, or 5;
each u is independently 1 or 2;
R²⁰ and R²¹ are each independently H; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, —NO₂, R⁴⁶ or C(O)R⁴⁷; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₃-C₈cycloalkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; 4- to 7-membered monocyclic heterocycle group which is unsubstituted or is substituted with from 1 to 3 substituents selected from C₁-C₈acyl, C₁-C₆alkyl, heterocyclylalkyl, (C₆-C₁₄aryl)alkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂; or R²⁰ and R²¹ when taken together with the nitrogen to which they are attached can form a 3- to 7-membered nitrogen containing heterocycle wherein up to two of the carbon atoms of the heterocycle can be replaced with —N(R²³)—, —O—, or —S(O)ᵤ—;
R²² is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; or C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂;
R²³ is hydrogen; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₃-C₈cycloalkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; amino(C₁-C₆alkyl)-; or arylamino;
R¹⁹ is hydrogen; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; heterocyclylalkyl; 4- to 7-membered monocyclic heterocycle group which is unsubstituted or is substituted with from 1 to 3 substituents independently selected from C₁-C₈acyl, C₁-C₆alkyl, heterocyclylalkyl, wherein the ring portion of the heterocyclylalkyl group is unsubstituted or is substituted by 1 to 3 substituents independently selected from halogen, —NH₂, —O(C₁-C₆alkyl), C₁-C₆alkyl, 4- to 7-membered monocyclic heterocycle, and C₃-C₈cycloalkyl, (C₆-C₁₄aryl)alkyl, wherein the ring portion of the (C₆-C₁₄aryl)alkyl group is unsubstituted or is substituted by 1 to 3 substituents independently selected from halogen, —NH₂, —O(C₁-C₆alkyl), C₁-C₆alkyl, 4- to 7-membered monocyclic heterocycle, and C₃-C₈cycloalkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂; or C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂;
R⁴⁶ is piperazinyl optionally substituted with 1 or 2 C₁-C₆alkyl; —O(C₂-C₃alkylene)N(C₁-C₆alkyl)(C₁-C₆alkyl); or —(C₁-C₃alkylene)N(C₁-C₆alkyl)(C₁-C₆alkyl); and
R⁴⁷ is piperazinyl optionally substituted with 1 or 2 C₁-C₆alkyl; or —N(C₁-C₃alkyl)-C₂-C₃alkylene-N(C₁-C₆alkyl)(C₁-C₆alkyl).

37. The compound of claim 36, wherein s is 0.

38. The compound of claim 36, wherein t is 1.

39. The compound of claim 36, wherein A is —CH₂—O—.

40. The compound of claim 36, wherein the dashed line - - - - - represents a second carbon-to-carbon bond.

41. The compound of claim 36, wherein R¹⁸ is —NHC(O)NR²⁰R²¹.

42. The compound of claim 41, wherein R²⁰ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂.

43. The compound of claim 42, wherein R²⁰ is methyl.

44. The compound of claim 42, wherein R²⁰ is 1-fluoroethyl.

45. The compound of claim 42, wherein R²⁰ is phenyl.

46. The compound of claim 42, wherein R²⁰ is 3-pyridyl.

47. The compound of claim 41, wherein R²¹ is H.

48. The compound of claim 36, wherein R¹⁹ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂.

49. The compound of claim 48, wherein R¹⁹ is 1,1 μl,-trifluoroethyl.

50. The compound of claim 36, wherein B is CH.

51. The compound of claim 36, wherein s is 0; t is 1; A is —CH₂—O—; the dashed line - - - - - represents a second carbon to carbon bond; R¹⁸ is —NHC(O)NR²⁰R²¹; R²⁰ is selected from the group consisting of methyl, 1-fluoroethyl, phenyl, and 3-pyridyl; R²¹ is H; R¹⁹ is 1,1,1,-trifluoroethyl; and B is CH.

52. A compound selected from the group consisting of:
3-[7-(3,6-dihydro-2H-pyran-4-yl)-3H-[1,2,3]triazolo[4,5-d]pyrimidin-5-yl]phenol;
{3-[7-(3,6-dihydro-2H-pyran-4-yl)-3H-[1,2,3]triazolo[4,5-d]pyrimidin-5-yl]phenyl}methanol;
5-[7-(3,6-dihydro-2H-pyran-4-yl)-3H-[1,2,3]triazolo[4,5-d]pyrimidin-5-yl]pyridin-3-ol;
3-[3-(1-benzylpiperidin-4-yl)-7-(3,6-dihydro-2H-pyran-4-yl)-3H-[1,2,3]triazolo[4,5-d]pyrimidin-5-yl]phenol;
4-(3-(4-(7-(3,6-dihydro-2H-pyran-4-yl)-3-ethyl-3H-[1,2,3]triazolo[4,5-d]pyrimidin-5-yl)phenyl)ureido)-N-(2-(dimethylamino)ethyl)-N-methylbenzamide;
1-(4-(7-(3,6-dihydro-2H-pyran-4-yl)-3-ethyl-3H-[1,2,3]triazolo[4,5-d]pyrimidin-5-yl)phenyl)-3-(4-(4-methylpiperazine-1-carbonyl)phenyl)urea;
1-(4-(7-(3,6-dihydro-2H-pyran-4-yl)-3-ethyl-3H-[1,2,3]triazolo[4,5-d]pyrimidin-5-yl)phenyl)-3-(4-(4-methylpiperazin-1-yl)phenyl)urea; and
1-(4-(7-(3,6-dihydro-2H-pyran-4-yl)-3-ethyl-3H-[1,2,3]triazolo[4,5-d]pyrimidin-5-yl)phenyl)-3-(pyridin-4-yl)urea.

53. A compound of the Formula 5: 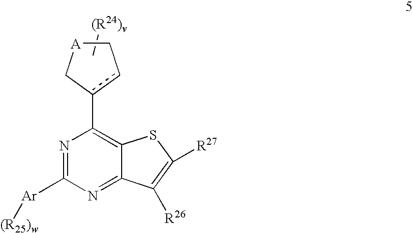
or a pharmaceutically acceptable salt thereof, wherein
A is —O—, —CH₂—O—, —CH₂—CH₂—O—, —CH₂—O—CH₂—, or —CH₂—S—;
the dashed line - - - - - represents an optional second carbon to carbon bond;
R²⁴ is C₁-C₆alkyl; C₂-C₆alkenyl; C₂-C₆alkynyl; or C₃-C₈cycloalkyl each of which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)C₁-C₆alkyl, C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂;
v is 0, 1, or 2;
Ar is phenyl, naphthyl, or nitrogen-containing mono- or bicyclic heteroaryl;
R²⁵ is independently halogen; one of the meanings of R²⁴; C₁-C₆alkoxy which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₁-C₆alkoxycarbonyl; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; hydroxyl; NR²⁸R²⁹; NO₂; CN; —C(O)NR²⁸R²⁹; R³⁰C(O)NH—; CO₂H; CF₃; CF₃O; C₁-C₆alkylthio; —SO₂NR²⁸R²⁹; —NHC(O)NR²⁸R²⁹; —NHC(O)OR³⁰; —NH(SO₂)NH—(C₁-C₆alkyl); —NH(SO₂)NH—(C₆-C₁₄aryl); —NHC(S)—NH—(C₁-C₆alkyl); —N═C(S—(C₁-C₆alkyl))(NH—(C₁-C₆alkyl)); —S(O)ₓ—(C₆-C₁₄aryl); —S(O), —(C₁-C₉heteroaryl); or —N(H)—C(═N—(CN))—(O—(C₆-C₁₄aryl));
w is 0, 1, 2, 3, 4, or 5;
each x is independently 1 or 2;
R²⁸ and R²⁹ are each independently H; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, —NO₂, R⁴⁶ or C(O)R⁴⁷; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₃-C₈cycloalkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; 4- to 7-membered monocyclic heterocycle group which is unsubstituted or is substituted with from 1 to 3 substituents selected from C₁-C₈acyl, C₁-C₆alkyl, heterocyclylalkyl, (C₆-C₁₄aryl)alkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂; or R²⁸ and R²⁹ when taken together with the nitrogen to which they are attached can form a 3- to 7-membered nitrogen containing heterocycle wherein up to two of the carbon atoms of the heterocycle can be replaced with —N(R³¹)—, —O—, or —S(O)ₓ—;
R³⁰ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; or C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂;
R³¹ is hydrogen; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₃-C₈cycloalkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; amino(C₁-C₆alkyl)-; or arylamino;
R²⁶ and R²⁷ independently are hydrogen; or are C₁-C₆alkyl; C₂-C₆alkenyl; or C₂-C₆alkynyl each of which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂;
R⁴⁶ is piperazinyl optionally substituted with 1 or 2 C₁-C₆alkyl; —O(C₂-C₃alkylene)N(C₁-C₆alkyl)(C₁-C₆alkyl); or —(C₁-C₃alkylene)N(C₁-C₆alkyl)(C₁-C₆alkyl); and
R⁴⁷ is piperazinyl optionally substituted with 1 or 2 C₁-C₆alkyl; or —N(C₁-C₃alkyl)-C₂-C₃alkylene-N(C₁-C₆alkyl)(C₁-C₆alkyl).

54. The compound of claim 53, wherein v is 0.

55. The compound of claim 53, wherein w is 1.

56. The compound of claim 53 wherein A is —CH₂—O—.

57. The compound of claim 53, wherein the dashed line - - - - - represents a second carbon-to-carbon bond.

58. The compound of claim 53, wherein R²⁵ is —NHC(O)NR²⁸R²⁹.

59. The compound of claim 58, wherein R²⁸ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂.

60. The compound of claim 59, wherein R²⁸ is methyl.

61. The compound of claim 59, wherein R²⁸ is 1-fluoroethyl.

62. The compound of claim 59, wherein R²⁸ is phenyl.

63. The compound of any of claim 59, wherein R²⁸ is 3-pyridyl.

64. The compound of claim 58, wherein R²⁹ is H.

65. The compound of claim 53, wherein Ar is phenyl.

66. The compound of claim 53, wherein R²⁶ is H.

67. The compound of claim 53, wherein R²⁷ is H.

68. The compound of claim 53, wherein v is 0; w is 1; A is —CH₂—O—; the dashed line - - - - - represents a second carbon to carbon bond; R²⁵ is —NHC(O)NR²⁸R²⁹; R²⁸ is selected from the group consisting of methyl, 1-fluoroethyl, phenyl, and 3-pyridyl; R⁶ is H; Ar is phenyl; and R²⁶ is H.

69. A compound selected from the group consisting of:
1-(4-(4-(3,6-dihydro-2H-pyran-4-yl)thieno[2,3-d]pyrimidin-2-yl)phenyl)-3-ethylurea;
1-(4-(4-(3,6-dihydro-2H-pyran-4-yl)thieno[2,3-d]pyrimidin-2-yl)phenyl)-3-(2-fluoroethyl)urea;
1-(4-(4-(3,6-dihydro-2H-pyran-4-yl)thieno[2,3-d]pyrimidin-2-yl)phenyl)-3-phenylurea;
1-(4-(4-(3,6-dihydro-2H-pyran-4-yl)thieno[2,3-d]pyrimidin-2-yl)phenyl)-3-(pyridin-3-yl)urea;
1-(4-(4-(3,6-dihydro-2H-pyran-4-yl)thieno[2,3-d]pyrimidin-2-yl)phenyl)-3-(pyridin-4-yl)urea;
4-(4-(3,6-dihydro-2H-pyran-4-yl)thieno[2,3-d]pyrimidin-2-yl)aniline;
N-(4-(4-(3,6-dihydro-2H-pyran-4-yl)thieno[2,3-d]pyrimidin-2-yl)phenyl)acetamide;
methyl 4-(4-(3,6-dihydro-2H-pyran-4-yl)thieno[2,3-d]pyrimidin-2-yl)phenylcarbamate;
3-(4-(3,6-dihydro-2H-pyran-4-yl)thieno[2,3-d]pyrimidin-2-yl)phenol;
4-(4-(3,6-dihydro-2H-pyran-4-yl)thieno[2,3-d]pyrimidin-2-yl)phenol;
4-(3,6-dihydro-2H-pyran-4-yl)-2-(1H-indol-5-yl)thieno[2,3-d]pyrimidine;
5-(4-(3,6-dihydro-2H-pyran-4-yl)thieno[2,3-d]pyrimidin-2-yl)pyridin-2-amine;
2-hydroxyethyl 4-(4-(3,6-dihydro-2H-pyran-4-yl)thieno[3,2-d]pyrimidin-2-yl)phenylcarbamate;
1-(4-(4-(3,6-dihydro-2H-pyran-4-yl)thieno[3,2-d]pyrimidin-2-yl)phenyl)-3-(4-(4-methylpiperazin-1-yl)phenyl)urea;
1-(pyridin-3-yl)-3-(4-(4-(tetrahydro-2H-pyran-4-yl)thieno[3,2-d]pyrimidin-2-yl)phenyl)urea;
2-hydroxyethyl 4-(4-(3,6-dihydro-2H-pyran-4-yl)thieno[3,2-d]pyrimidin-2-yl)phenylcarbamate;
1-(4-(4-(3,6-dihydro-2H-pyran-4-yl)thieno[3,2-d]pyrimidin-2-yl)phenyl)-3-(4-(4-methylpiperazin-1-yl)phenyl)urea; and
1-(pyridin-3-yl)-3-(4-(4-(tetrahydro-2H-pyran-4-yl)thieno[3,2-d]pyrimidin-2-yl)phenyl)urea.

70. A compound of the Formula 6: 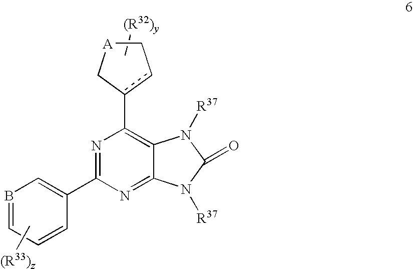
or a pharmaceutically acceptable salt thereof, wherein
A is —O—, —CH₂—O—, —CH₂—CH₂—O—, —CH₂—O—CH₂—, or —CH₂—S—;
the dashed line - - - - - represents an optional second carbon to carbon bond;
R³² is independently C₁-C₆alkyl; C₂-C₆alkeny; C₂-C₆alkynyl; or C₃-C₈cycloalkyl each of which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)C₁-C₆alkyl, C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂;
y is 0, 1, or 2;
B is N or CH;
R³³ is independently halogen; one of the meanings of R³²; C₁-C₆alkoxy which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₁-C₆alkoxycarbonyl; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; hydroxyl; NR³⁴R³⁵; NO₂; CN; —C(O)NR³⁴R³⁵; R³⁶C(O)NH—; CO₂H; CF₃; CF₃O; C₁-C₆alkylthio; —SO₂NR³⁴R³⁵; —NHC(O)NR³⁴R³⁵; —NHC(O)OR³⁶; —NH(SO₂)NH—(C₁-C₆alkyl); —NH(SO₂)NH—(C₆-C₁₄aryl); —NHC(S)—NH—(C₁-C₆alkyl); —N═C(S—(C₁-C₆alkyl))(NH—(C₁-C₆alkyl)); —S(O)ₐ—(C₆-C₁₄aryl); —S(O)ₐ—(C₁-C₉heteroaryl); or —N(H)—C(═N—(CN))—(O—(C₆-C₁₄aryl));
z is 0, 1, 2, 3, 4, or 5;
each a is independently 1 or 2;
R³⁴ and R³⁵ are each independently H; C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, —NO₂, R⁴⁶ or C(O)R⁴⁷; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₃-C₈cycloalkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; 4- to 7-membered monocyclic heterocycle group which is unsubstituted or is substituted with from 1 to 3 substituents selected from C₁-C₈acyl, C₁-C₆alkyl, heterocyclylalkyl, (C₆-C₁₄aryl)alkyl, halo, halo(C₁-C₆alkyl)-, hydroxyl, hydroxyl(C₁-C₆alkyl)-, —NH₂, aminoalkyl-, -dialkylamino-, —COOH, —C(O)O—(C₁-C₆alkyl), —OC(O)(C₁-C₆alkyl), (C₆-C₁₄)arylalkyl-O—C(O)—, N-alkylamido-, —C(O)NH₂, (C₁-C₆alkyl)amido-, or —NO₂; or R³⁴ and R³⁵ when taken together with the nitrogen to which they are attached can form a 3- to 7-membered nitrogen containing heterocycle wherein up to two of the carbon atoms of the heterocycle can be replaced with —NH—, —O—, or —S(O)ₐ—;
R³⁶ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; or C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂;
R³⁷ are independently C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂;
R⁴⁶ is piperazinyl optionally substituted with 1 or 2 C₁-C₆alkyl; —O(C₂-C₃alkylene)N(C₁-C₆alkyl)(C₁-C₆alkyl); or —(C₁-C₃alkylene)N(C₁-C₆alkyl)(C₁-C₆alkyl); and
R⁴⁷ is piperazinyl optionally substituted with 1 or 2 C₁-C₆alkyl; or —N(C₁-C₃alkyl)-C₂-C₃alkylene-N(C₁-C₆alkyl)(C₁-C₆alkyl).

71. The compound of claim 70, wherein y is 0.

72. The compound of claim 70, wherein z is 1.

73. The compound of claim 70 wherein A is —CH₂—O—.

74. The compound of claim 70, wherein the dashed line - - - - - represents a second carbon-to-carbon bond.

75. The compound of claim 70, wherein R³³ is —NHC(O)NR³⁴R³⁵.

76. The compound of claim 75, wherein R³⁴ is C₁-C₆alkyl which is unsubstituted or is substituted with from 1 to 3 substituents independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, halo(C₁-C₆alkyl)-, amino(C₁-C₆alkyl)-, —OC(O)(C₁-C₆alkyl), carboxyamidoalkyl-, or —NO₂; C₆-C₁₄aryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂; C₁-C₉heteroaryl which is unsubstituted or is substituted with from 1 to 3 substituents independently selected from C₁-C₆alkyl, halo, halo(C₁-C₆alkyl)-, hydroxyl, hydroxyl(C₁-C₆alkyl)-, —NH₂, amino(C₁-C₆alkyl)-, alkylamino-, di(C₁-C₆alkyl)amino-, —COOH, —C(O)O—(C₁-C₆alkyl), —OC(O)(C₁-C₆alkyl), (C₁-C₆alkyl)carboxyamido-, —C(O)NH₂, alkylcarboxamido-, or —NO₂.

77. The compound of claim 76, wherein R³⁴ is methyl.

78. The compound of claim 76, wherein R³⁴ is 1-fluoroethyl.

79. The compound of claim 76 wherein R³⁴ is phenyl.

80. The compound of claim 76, wherein R³⁴ is 3-pyridyl.

81. The compound of claim 75, wherein R³⁵ is H.

82. The compound of claim 70, wherein R³⁷ is C₁-C₆alkyl, which is unsubstituted or is substituted with from 1 to 3 substituents independently selected from halogen and C₆-C₁₄aryl.

83. The compound of claim 82 wherein R³⁷ both are 1,1,1,-trifluoroethyl.

84. The compound of claim 82, wherein R³⁷ both are benzyl.

85. The compound of claim 70 wherein y is 0; z is 1; A is —CH₂—O—; the dashed line - - - - - represents a second carbon to carbon bond; R³³ is —NHC(O)NR³⁴R³⁵; R³⁴ is selected from the group consisting of methyl, 1-fluoroethyl, phenyl, and 3-pyridyl; R³⁵ is H; R³⁷ both are 1,1,1,-trifluoroethyl; and B is CH.

86. The compound 7,9-dibenzyl-6-(3,6-dihydro-2H-pyran-4-yl)-2-(3-hydroxyphenyl)-7,9-dihydro-8H-purin-8-one.

87. A composition comprising the compound of claim 1, and a pharmaceutically acceptable carrier.

88. The composition of claim 87, wherein the pharmaceutically acceptable carrier is suitable for oral administration and the composition comprises an oral dosage form.

89. A method of inhibiting mTOR, comprising administering to a mammal the compound of claim 1 in an effective amount.

90. A method of inhibiting PI3K, comprising administering to a mammal the compound of claim 1 in an effective amount.

91. A method of treating advanced renal cell carcinoma, comprising administering to a mammal in need thereof an effective amount of the compound of claim 1.

92. A method of treating acute lymphoblastic leukemia, comprising administering to a mammal in need thereof an effective amount of the compound of claim 1.

93. A method of treating malignant melanoma, comprising administering to a mammal in need thereof an effective amount of the compound of claim 1.

94. A method of treating soft-tissue or bone sarcoma, comprising administering to a mammal in need thereof an effective amount of the compound of claim 1.

95. A method of synthesizing a compound of claim 2 comprising reacting a chloro 1H-pyrazolo[3,4-d]pyrimidine of Formula 12 with 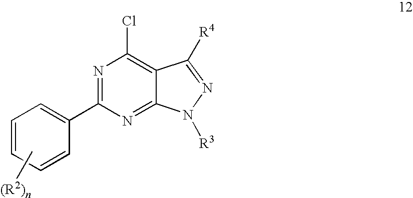
a tributylstannane compound 13: 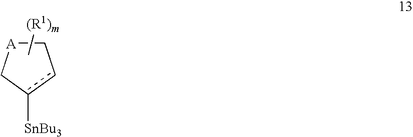
wherein A, the dashed line - - - - -, R¹, R², R³, R⁴, m, and n are as defined in claim 2.

96. The method of claim 95 further comprising a) reacting a hydrazine of the formula H₂N—NH—R³ with the nitrile 8 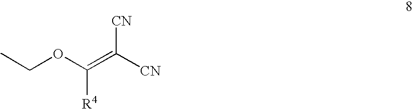
to give the aminopyrazole 9: 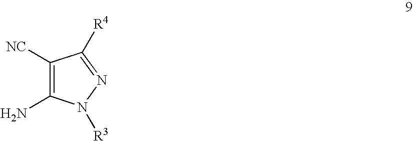
(b) reacting the amino pyrazole of Formula 9 with an acid chloride compound 10: 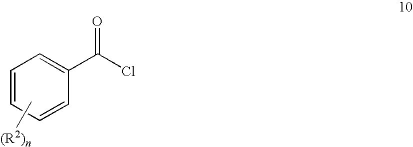
thereby producing amide 11; 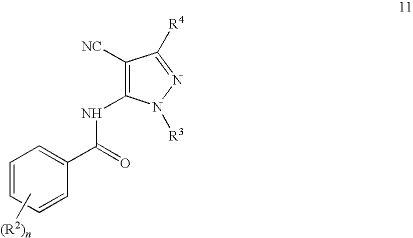
(c) cyclizing the amide 11 under oxidizing conditions and chlorinating the newly formed lactam to introduce a chlorine atom at position 4 of the 1H-pyrazolo[3,4-d]pyrimidine: 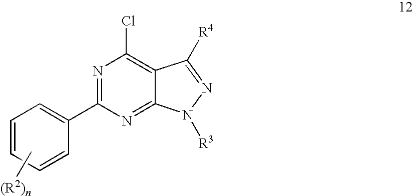
thereby producing the chlorinated intermediate 12.

97. The method of claim 96 further comprising reducing the double bond of the 1H-pyrazolo[3,4-d]pyrimidine of Formula 2: 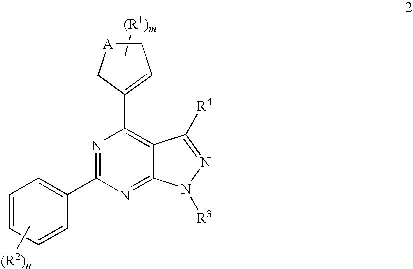
thereby producing the 1H-pyrazolo[3,4-d]pyrimidine of Formula 14: 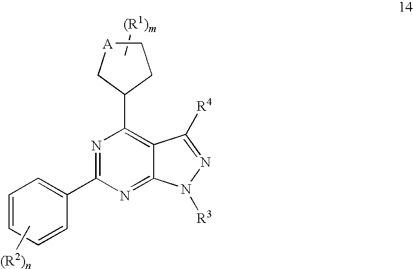
or a pharmaceutically acceptable salt thereof.

98. A method of synthesizing a compound of claim 19 comprising reacting the chloropurine of Formula 18 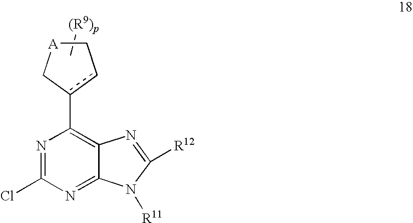
with a boronic acid compound 19, 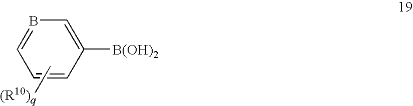
wherein A, the B in the aromatic ring, the dashed line - - - - -, R⁹, R¹⁰, R¹¹, R¹², p, and q are as defined in claim 19.

99. The method of claim 98 further comprising: a) reacting a 2,4-dichloropurine of the Formula 15 with the alcohol R¹¹OH; 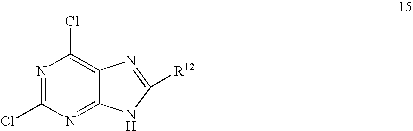
thereby providing a compound of the Formula 16: 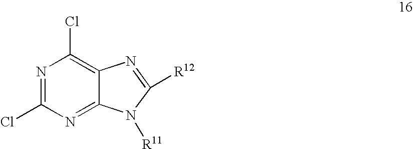
and (b) reacting the dichloro purine of Formula 16 with a tributylstannane compound 17: 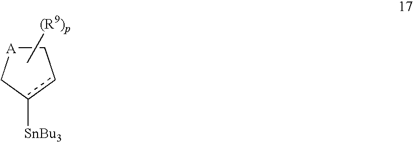
thereby replacing the chlorine atom at position 6 of the purine ring.

100. A method of synthesizing a compound of claim 36 comprising employing a monochloro compound of the Formula 24: 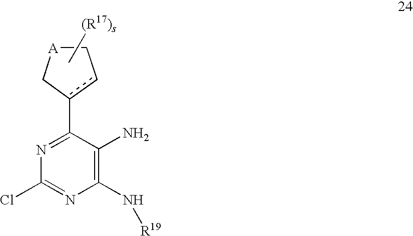
and performing either a two step sequence of Suzuki coupling with the boronic acid 25 followed by diazotization and cyclization or a two step sequence of diazotization and cyclization followed by Suzuki coupling with the boronic acid 25: 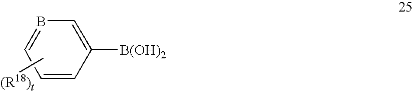
wherein A, the B in the aromatic ring, the dashed line - - - - -, R¹⁷, R¹⁸, R¹⁹, s, and t are as defined in claim 36 thereby substituting the chlorine atom at position 2 of the pyrimidine ring with the aromatic radical from the boronic acid.

101. The method of claim 100 further comprising a) reacting 5-nitro-2,4,6-trichloropyrimidine of the Formula 20 with the amine R¹⁹NH₂; 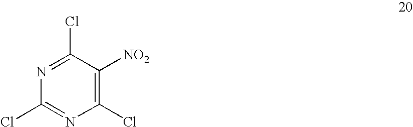
to give the dichloropyrimidine intermediate of Formula 21: 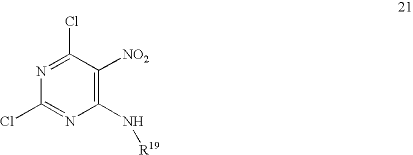
(b) reacting the dichloro compound of Formula 21 with a tributylstannane compound 22 thereby providing a compound of the Formula 23: 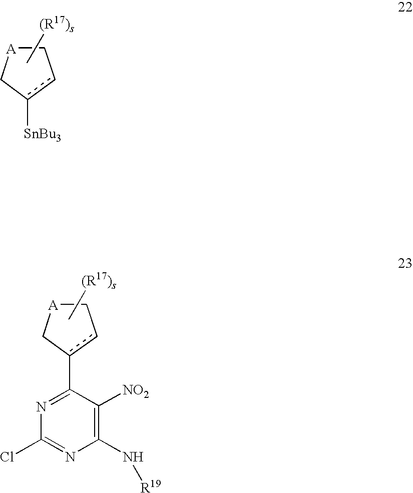
c) reducing the compound of Formula 23 thereby providing compound 24.

102. A method of synthesizing a compound of claim 53 comprising reacting the compound of Formula 30 with a boronic acid of the structure (R²⁵)_{w}—ArB(OH)₂: 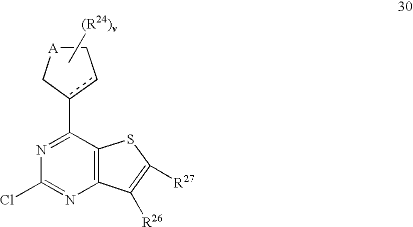
wherein A, the dashed line - - - - -, Ar, R²⁴, R²⁵, R²⁶, R²⁷, v, and w are as defined in claim 53.

103. The method of claim 102 further comprising: a) reacting a 2-amido-3-amino-thiophene of the Formula 26: 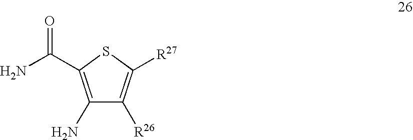
with triphosgene to give the thieno[3,2-d]pyrimidine intermediate of Formula 27: 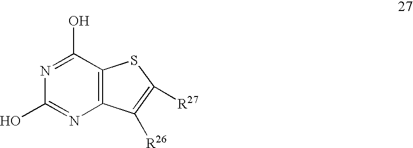
b) reacting the compound of Formula 27 with phosphorous oxychloride thereby providing a dichloro compound of the Formula 28: 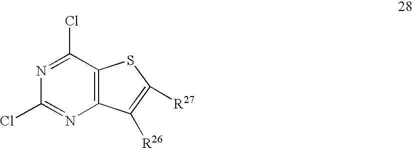
(c) reacting the dichloro compound of Formula 28 with a tributylstannane compound 29 to substitute the chlorine atom at position 4 of the thieno[3,2-d]pyrimidine compound 28 with the organic moiety from the tributylstannane 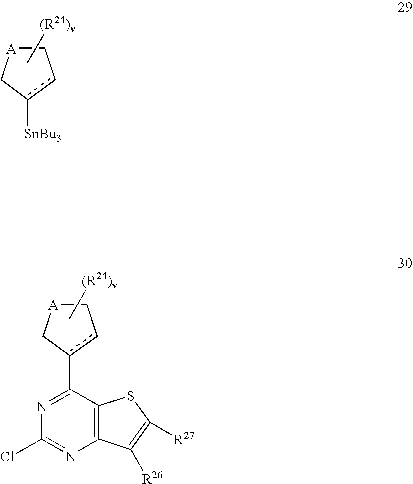
thereby providing a compound of the Formula 30.

104. A method of synthesizing a compound of claim 70 comprising reacting the compound of Formula 31: 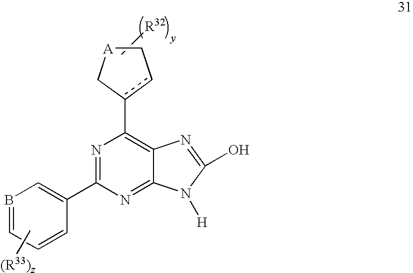
with a base and an alkylating agent R³⁷X wherein A, the dashed line - - - - -, R³², y, B, R³³, z, and R³⁷ are as defined in claim 70 and X is halogen.
